(19) United States
(12) Patent Application Publication
(10) Pub. No.: US 2017/0037105 A1
(43) Pub. Date: Feb. 9, 2017

(54) PEPTIDOMIMETIC MACROCYCLES

(71) Applicant: Aileron Therapeutics, Inc., Cambridge, MA (US)

(72) Inventor: Manoj Samant, Burlington, MA (US)

(21) Appl. No.: 15/226,059

(22) Filed: Aug. 2, 2016

Related U.S. Application Data
(60) Provisional application No. 62/200,227, filed on Aug. 3, 2015.
(60) Provisional application No. 62/235,621, filed on Oct. 1, 2015.
(60) Provisional application No. 62/260,753, filed on Nov. 30, 2015.

(51) Int. Cl. C07K 14/60

(52) U.S. Cl. CPC C07K 14/60; A61K 38/00

(57) ABSTRACT

The present invention provides peptidomimetic macrocycles capable of modulating growth hormone levels and methods of using such macrocycles for the treatment of disease.

### CROSS REFERENCE

This application claims the benefit of U.S. Provisional Application No. 62/200,227, filed Aug. 3, 2015, U.S. Provisional Application No. 62/235,621, filed Oct. 1, 2015, and U.S. Provisional Application No. 62/260,753, filed Nov. 30, 2015, each of which are incorporated herein by reference in their entirety.

### BACKGROUND

Human GHRH (Growth Hormone-Releasing Hormone) is a 44-amino-acid peptide whose full biological activity resides in its first 29 amino acids (“GHRH 1-29”). GHRH binds to the GHRH receptor and stimulates pulsatile GH [Growth Hormone] secretion, and with this mechanism of action GHRH represents an alternative to GH therapy in patients with an intact pituitary that may minimize the side effects associated with long-term GH administration. Because the quantity of GH release induced by GHRH is limited by IGF-1 levels, which exert a negative feedback effect, the risk of side effects associated with excessive GH secretion may also be lower with GHRH therapy than with GH therapy. In addition, treatment with GHRH may result in the pituitary secretion of a broader set of GH proteins, and not just the 22-kDa form provided by recombinant human GH, which may also have beneficial effects. Clinically, GHRH has been shown to be safe and effective in increasing GH levels in adults and children, and the growth-promoting effect of GHRH is correlated with the dose and frequency of administration. However, the half-life of GHRH after intravenous injection is only 10-12 min, which has significantly limited its use as a therapeutic agent.

### SUMMARY

In some embodiments, the present invention provides a peptidomimetic macrocycle or a pharmaceutically-acceptable salt thereof comprising an amino acid sequence which is at least about 60%, 65%, 70%, 75%, 80%, 85%, 90%, 95%, 96%, 97%, 98%, 99%, or 100% identical to GHRH 1-29, and a macrocycle-forming linker connecting a first amino acid to a second amino acid, wherein the first and second amino acids are selected from amino acids corresponding to the following locations of amino acids: 2 and 9; 4 and 8; 5 and 12; 8 and 12; 8 and 15; 9 and 13; 12 and 16; 12 and 19; 13 and 17; 14 and 18; 14 and 21; 15 and 19; 15 and 22; 16 and 23; 17 and 21; 17 and 24; 18 and 22; 18 and 25; 19 and 23; 19 and 26; 21 and 25; 21 and 28; 22 and 26; 22 and 29; 23 and 27; 24 and 28; and 25 and 29; of amino acids 1-29 of Human Growth Hormone-Release Hormone (GHRH 1-29).

In some embodiments, the present invention provides a peptidomimetic macrocycle or a pharmaceutically-acceptable salt thereof comprising an amino acid sequence which is at least about 60%, 65%, 70%, 75%, 80%, 85%, 90%, 95%, 96%, 97%, 98%, 99%, or 100% identical to an amino acid sequence of Table 1a, 1b, 2a, 2b, or 2c, and a macrocycle-forming linker connecting a first amino acid to a second amino acid, wherein the first and second amino acids are selected from amino acids corresponding to the following locations of amino acids: 2 and 9; 4 and 8; 5 and 12; 8 and 12; 8 and 15; 9 and 13; 12 and 16; 12 and 19; 13 and 17; 14 and 18; 14 and 21; 15 and 19; 15 and 22; 16 and 23; 17 and 21; 17 and 24; 18 and 22; 18 and 25; 19 and 23; 19 and 26; 21 and 25; 21 and 28; 22 and 26; 22 and 29; 23 and 27; 24 and 28; and 25 and 29; of amino acids 1-29 of Human Growth Hormone-Release Hormone (GHRH 1-29).

In some embodiments, the present invention provides a peptidomimetic macrocycle or a pharmaceutically-acceptable salt thereof comprising an amino acid sequence, a PEG moiety, and a macrocycle-forming linker connecting a first amino acid to a second amino acid, wherein the peptidomimetic macrocycle or a pharmaceutically-acceptable salt thereof has a solubility of at least about 1 mg/ml, 5 mg/mL, 10 mg/mL, 25 mg/mL, 50 mg/mL, or 100 mg/mL.

In some embodiments, the present invention provides a peptidomimetic macrocycle or a pharmaceutically-acceptable salt thereof comprising an amino acid sequence, and a macrocycle-forming linker connecting a first amino acid to a second amino acid, wherein the peptidomimetic macrocycle or a pharmaceutically-acceptable salt thereof is attached to a ghrelin agonist, such as a ghrelin agonist of Table 3.

In some embodiments, the present invention provides a peptidomimetic macrocycle comprising an amino acid sequence with at least about 60%, 65%, 70%, 75%, 80%, 85%, 90%, 95%, 96%, 97%, 98%, 99% or 100% sequence identity to a sequence of Table 1a, 1b, 2a, 2b, or 2c, and having Formula (I):

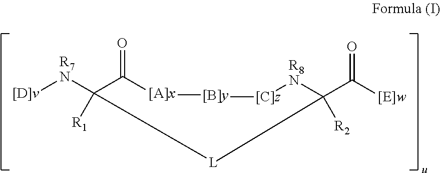

or a pharmaceutically-acceptable salt thereof, wherein:

each A, C, D, and E is independently an amino acid;

each B is independently an amino acid.

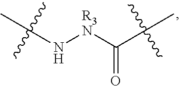

[—NH-L₃-CO—], [—NH-L₃-SO₂—], or [—NH-L₃-];

wherein A, B, C, D, and E, taken together with the crosslinked amino acids connected by the macrocycle-forming linker L, form the amino acid sequence of the peptidomimetic macrocycle;

each R₁ and R₂ is independently H, alkyl, alkenyl, alkynyl, arylalkyl, cycloalkyl, cycloalkylalkyl, heteroalkyl, or heterocycloalkyl, unsubstituted or substituted with halo; or at least one of R₁ and R₂ forms a macrocycle-forming linker L′ connected to the alpha position of one of the D or E amino acids;

each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅;

each L and L′ is independently a macrocycle-forming linker;

each L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;

each R₄ is independently alkylene, alkenylene, alkynylene, heteroalkylene, cycloalkylene, heterocycloalkylene, arylene, or heteroarylene;

each K is independently O, S, SO, SO₂, CO, CO₂ or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;

each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;

each v and w is independently an integer from 0-1000, for example 0-500, 0-200, 0-100, 0-50, 0-30, 0-20, or 0-10;

u is an integer from 1-10, for example 1-5, 1-3 or 1-2; and

each x, y and z is independently an integer from 0-10, for example the sum of x+y+z is 2, 3, 5, 6 or 10.

In some embodiments, the present invention provides a peptidomimetic macrocycle having Formula (Ia):

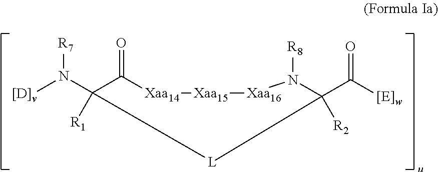

or a pharmaceutically-acceptable salt thereof, wherein:

each of Xaa₁₄, Xaa₁₅, and Xaa₁₆ is independently an amino acid, wherein at least one, two, or each of Xaa₁₄, Xaa₁₅, and Xaa₁₆ are the same amino acid as the amino acid at the corresponding position of the sequence Xaa₁₃-Leu₁₄-Ala/Gly/Abu₁₅-Gln/Ala/Glu/Nle/Ser₁₆-Xaa₁₇, where each of Xaa₁₃ and Xaa₁₇ is independently an amino acid;

each D and E is independently an amino acid;

each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of the D or E amino acids;

each L and L′ is independently a macrocycle-forming linker;

each R₃ is independently —H, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

each R₇ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;

each R₈ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;

each v is independently an integer from 1-1000;

each w is independently an integer from 1-1000; and

u is an integer from 1-100.

In some embodiments, the present invention provides a peptidomimetic macrocycle having Formula (Ib):

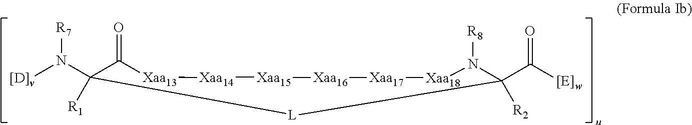

or a pharmaceutically-acceptable salt thereof, wherein:

each of Xaa₁₃, Xaa₁₄, Xaa₁₅, Xaa₁₆, Xaa₁₇, and Xaa₁₈ is independently an amino acid, wherein at least one, two, three, four, five, or each of Xaa₁₃, Xaa₁₄, Xaa₁₅, Xaa₁₆, Xaa₁₇, and Xaa₈, are the same amino acid as the amino acid at the corresponding position of the sequence Xaa₁₂-Val₁₃-Leu₁₄-Ala/Gly₁₅-Gln/Ala₁₆-Leu₁₇-Ser₁₈-Xaa₁₉, where each of Xaa₁₂ and Xaa₁₉ is independently an amino acid;

each D and E is independently an amino acid;

each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of the D or E amino acids;

each L and L′ is independently a macrocycle-forming linker;

each R₃ is independently —H, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

each R₇ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;

each R₈ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;

each v is independently an integer from 1-1000;

each w is independently an integer from 1-1000; and

u is an integer from 1-100.

In some embodiments, the present invention provides a peptidomimetic macrocycle or a pharmaceutically-acceptable salt thereof comprising an amino acid sequence of formula

Xaa0-Xaa1-Xaa2-Xaa3-Xaa4-Xaa5-Xaa6-Xaa7-Xaa8-Xaa9-
Xaa10-Xaa11-Xaa12-Xaa13-Xaa14-Xaa15-Xaa16-Xaa17-
Xaa18-Xaa19-Xaa20-Xaa21-Xaa22-Xaa23-Xaa24-Xaa25-
Xaa26-Xaa27-Xaa28-Xaa29-Xaa30-Xaa31-Xaa32-Xaa33-
Xaa34-Xaa35

wherein:

Xaa0 is —H or an N-terminal capping group;

Xaa35 is —OH, or a C-terminal capping group;

Xaa1, Xaa2, Xaa3, Xaa31, Xaa32, Xaa33 and Xaa34 are independently absent, a spacer (such as PEG), or an amino acid (such as Lys) that is optionally conjugated;

wherein the peptidomimetic macrocycle comprises at least one macrocycle-forming linker connecting at least one pair of amino acids selected from Xaa2-Xaa31, and wherein Xaa1-Xaa34 together with the crosslinked amino acids, form an amino acid sequence with at least about 60%, 65%, 70%, 75%, 80%, 85%, 90%, 95%, 96%, 97%, 98%, 99% or 100% sequence identity to a sequence of Table 1a, 1b, 2a, 2b, or 2c.

In some embodiments, the present invention provides a peptidomimetic macrocycle or a pharmaceutically-acceptable salt thereof comprising an amino acid sequence of formula:

Xaa0-[D]_{V}-Xaa1-Xaa2-Xaa3-Xaa4-Xaa5-Xaa6-Xaa7-
Xaa8-Xaa9-Xaa10-Xaa11-Xaa12-Xaa13-Xaa14-Xaa15-
Xaa16-Xaa17-Xaa18-Xaa19-Xaa20-Xaa21-Xaa22-Xaa23-
Xaa24-Xaa25-Xaa26-Xaa27-Xaa28-Xaa29-Xaa30-Xaa31-
Xaa32-Xaa33-Xaa34-[E]_{W}-Xaa35,

wherein:

Xaa0 is —H or an N-terminal capping group;

Xaa1 is absent or Pro;

Xaa2 is absent, a crosslinked amino acid, K(γ-Glu-C₁₈-dicarboxylic acid), or Pro;

Xaa3 is absent, Tyr, F₄COOH, F₄NH₂, NipY, or NmY;

Xaa4 is Ala, D-Ala, Ile, or a crosslinked amino acid;

Xaa5 is Asp or Pro;

Xaa6 is Ala or a crosslinked amino acid;

Xaa7 is Ile or a crosslinked amino acid;

Xaa8 is Phe or a conjugated Lys;

Xaa9 is Thr or a conjugated Lys;

Xaa10 is Ala, Gln, Asn, Aib, Thr or a crosslinked amino acid;

Xaa11 is Ser or a crosslinked amino acid;

Xaa12 is Tyr;

Xaa13 is Arg or Cit;

Xaa14 is Lys, ipK or a crosslinked amino acid;

Xaa15 is Val, a conjugated Lys, or a crosslinked amino acid;

Xaa16 is Leu, a conjugated Lys, or a crosslinked amino acid;

Xaa17 is Gly, Abu, Ala or a crosslinked amino acid;

Xaa18 is Ala, Nle, Ser, Gln, Glu, a conjugated Lys, or a crosslinked amino acid;

Xaa19 is Leu, a conjugated Lys, or a crosslinked amino acid;

Xaa20 is Ser, Aib or a crosslinked amino acid;

Xaa21 is Ala or a crosslinked amino acid;

Xaa22 is Arg, Cit, a conjugated Lys, or a crosslinked amino acid;

Xaa23 is Lys, ipK or a crosslinked amino acid;

Xaa24 is Leu, Ala, Aib, a conjugated Lys, or a crosslinked amino acid;

Xaa25 is Leu a conjugated Lys, or a crosslinked amino acid;

Xaa26 is Gln, Ala, Aib, a conjugated Lys, or a crosslinked amino acid;

Xaa27 is Asp, Ala or a crosslinked amino acid;

Xaa28 is Ile, Ala, a conjugated Lys, or a crosslinked amino acid;

Xaa29 is Ala, Hse(Me), Nle or a crosslinked amino acid;

Xaa30 is Ser, Asp or a crosslinked amino acid;

Xaa31 is absent, Arg, Cit or a crosslinked amino acid;

Xaa32 is absent, Glu, a conjugated Lys, or a PEG;

Xaa33 is absent, Glu, or a PEG;

Xaa34 is absent, Glu, or a PEG; and

Xaa35 is —NH₂ or —OH;

wherein each of D and E are independently an amino acid;

each of v and w is independently an integer from 1-100; and

wherein the peptidomimetic macrocycle comprises at least one macrocycle-forming linker connecting at least one pair of amino acids selected from Xaa1-Xaa32.

In some embodiments, the present invention provides a method of increasing the circulating level of growth hormone (GH) in a subject comprising administering to the subject a peptidomimetic macrocycle of the invention.

In some embodiments, the present invention provides a method of increasing lean muscle mass in a subject comprising administering to the subject a peptidomimetic macrocycle of the invention.

In some embodiments, the present invention provides a method of reducing adipose tissue in a subject comprising administering to the subject a peptidomimetic macrocycle of the invention.

In some embodiments, the present invention provides a method of treating muscle wasting diseases, including anorexias, cachexias (such as cancer cachexia, chronic heart failure cachexia, chronic obstructive pulmonary disease cachexia, rheumatoid arthritis cachexia, cachexia in liver cirrohsis) or sarcopenias in a subject comprising administering to the subject a peptidomimetic macrocycle of the invention.

In some embodiments, the present invention provides a method of treating lipodystrophies, including HIV lipodystrophy, in a subject comprising administering to the subject a peptidomimetic macrocycle of the invention.

In some embodiments, the present invention provides a method of treating a growth hormone disorder in a subject comprising administering to the subject a peptidomimetic macrocycle of the invention.

In some embodiments, the present invention provides a method of treating gastroparesis or short bowel syndrome in a subject comprising administering to the subject a peptidomimetic macrocycle of the invention.

In some embodiments, the present invention provides a method of treating muscle wasting diseases, lipodystrophies, growth hormone disorders or gastroparesis/short bowel syndrome in a subject by administering a peptidomimetic macrocycle of the invention, wherein the peptidomimetic macrocycle is administered no more frequently than once daily, no more frequently than every other day, no more frequently than twice weekly, no more frequently than weekly, or no more frequently than every other week.

In some embodiments, the present invention provides a method of treating muscle wasting diseases, lipodystrophies, growth hormone disorders or gastroparesis/short bowel syndrome in a subject by administering a peptidomimetic macrocycle of the invention, wherein the peptidomimetic macrocycle is administered no more frequently than once daily, no more frequently than every other day, no more frequently than twice weekly, no more frequently than weekly, or no more frequently than every other week.

In some embodiments, the present invention provides a method of increasing the circulating level of growth hormone (GH) in a subject by administering a peptidomimetic macrocycle of the invention, wherein the peptidomimetic macrocycle is administered no more frequently than once daily, no more frequently than every other day, no more frequently than twice weekly, no more frequently than weekly, or no more frequently than every other week.

In some embodiments, the present invention provides a method of increasing the circulating level of growth hormone (GH) in a subject by administering a peptidomimetic macrocycle of the invention, wherein the peptidomimetic macrocycle is administered no more frequently than once daily, no more frequently than every other day, no more frequently than twice weekly, no more frequently than weekly, or no more frequently than every other week.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

As used herein, the term “macrocycle” refers to a molecule having a chemical structure including a ring or cycle formed by at least 9 covalently bonded atoms.

As used herein, the term “peptidomimetic macrocycle” or “crosslinked polypeptide” refers to a compound comprising a plurality of amino acid residues joined by a plurality of peptide bonds and at least one macrocycle-forming linker which forms a macrocycle between a first naturally-occurring or non-naturally-occurring amino acid residue (or analog) and a second naturally-occurring or non-naturally-occurring amino acid residue (or analog) within the same molecule. Peptidomimetic macrocycle include embodiments where the macrocycle-forming linker connects the α carbon of the first amino acid residue (or analog) to the α carbon of the second amino acid residue (or analog). The peptidomimetic macrocycles optionally include one or more non-peptide bonds between one or more amino acid residues and/or amino acid analog residues, and optionally include one or more non-naturally-occurring amino acid residues or amino acid analog residues in addition to any which form the macrocycle. A “corresponding uncrosslinked polypeptide” when referred to in the context of a peptidomimetic macrocycle is understood to relate to a polypeptide of the same length as the macrocycle and comprising the equivalent natural amino acids of the wild-type sequence corresponding to the macrocycle.

As used herein, the term “stability” refers to the maintenance of a defined secondary structure in solution by a peptidomimetic macrocycle as measured by circular dichroism, ¹HNMR or another biophysical measure, or resistance to proteolytic degradation in vitro or in vivo. Non-limiting examples of secondary structures contemplated herein are α-helices, 3₁₀ helices, β-turns, and β-pleated sheets.

As used herein, the term “helical stability” refers to the maintenance of α helical structure by a peptidomimetic macrocycle as measured by circular dichroism or NMR. For example, in some embodiments, a peptidomimetic macrocycle exhibits at least a 1.25, 1.5, 1.75 or 2-fold increase in α-helicity as determined by circular dichroism compared to a corresponding uncrosslinked macrocycle.

The term “amino acid” refers to a molecule containing both an amino group and a carboxyl group.

Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration. The abbreviation “b-” prior to an amino acid represents an amino acid whose side-chain is involved in lactam formation. For example, amino acids represented by “bK” and “bE” represent side-chain lactam formed between lysine and glutamic acid.

The term “naturally occurring amino acid” refers to any one of the twenty amino acids commonly found in peptides synthesized in nature, known by the one letter abbreviations A, R, N, C, D, Q, E, G, H, I, L, K, M, F, P, S, T, W, Y and V. The following table shows a summary of the properties of natural amino acids:

| | | | | Side-chain | Hydrop- |
|---|---|---|---|---|---|
| | 3-Letter | 1-Letter | Side-chain | charge | athy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive(10%) | −3.2 |
| | | | | neutral(90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acids” are glycine, alanine, proline, and analogs thereof. “Large hydrophobic amino acids” are valine, leucine, isoleucine, phenylalanine, methionine, tryptophan, tyrosine, and analogs thereof. “Polar amino acids” are serine, threonine, asparagine, glutamine, cysteine, and analogs thereof. “Charged amino acids” include positively charged amino acids and negatively charged amino acids. “Positively charged amino acids” include lysine, arginine, histidine, and analogs thereof. “Negatively charged amino acids” include aspartate, glutamate, and analogs thereof.

The term “amino acid analog” refers to a molecule which is structurally similar to an amino acid and which can be substituted for an amino acid in the formation of a peptidomimetic macrocycle. Amino acid analogs include, without limitation, β-amino acids and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include, without limitation, structures according to the following:

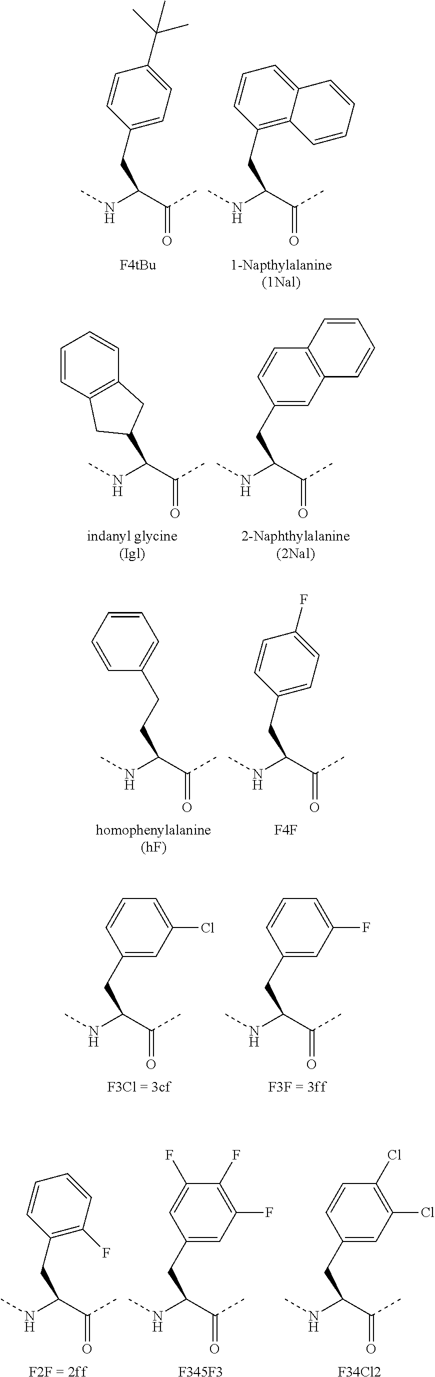

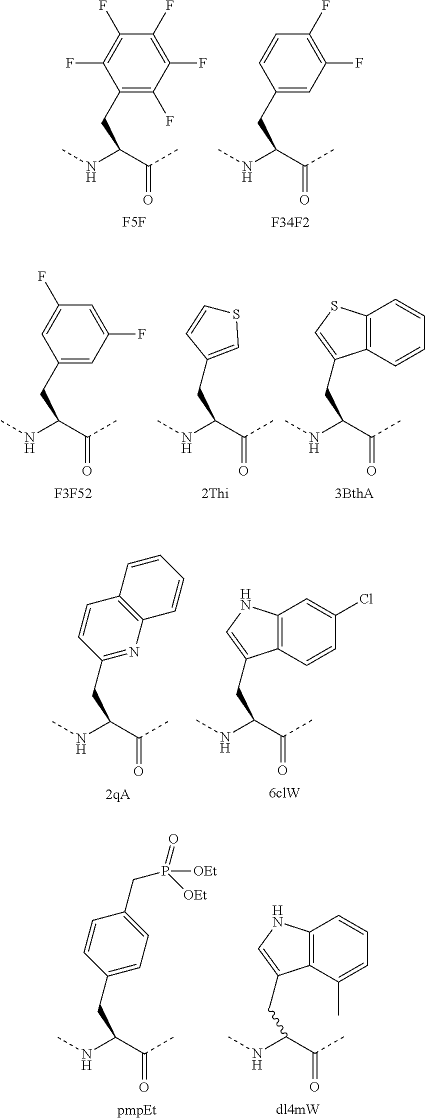

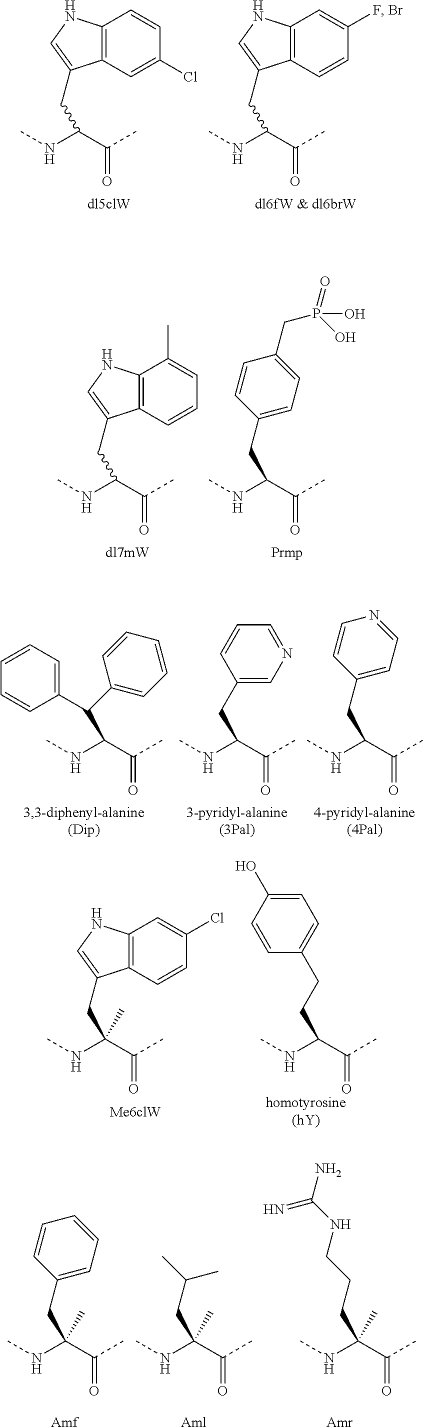

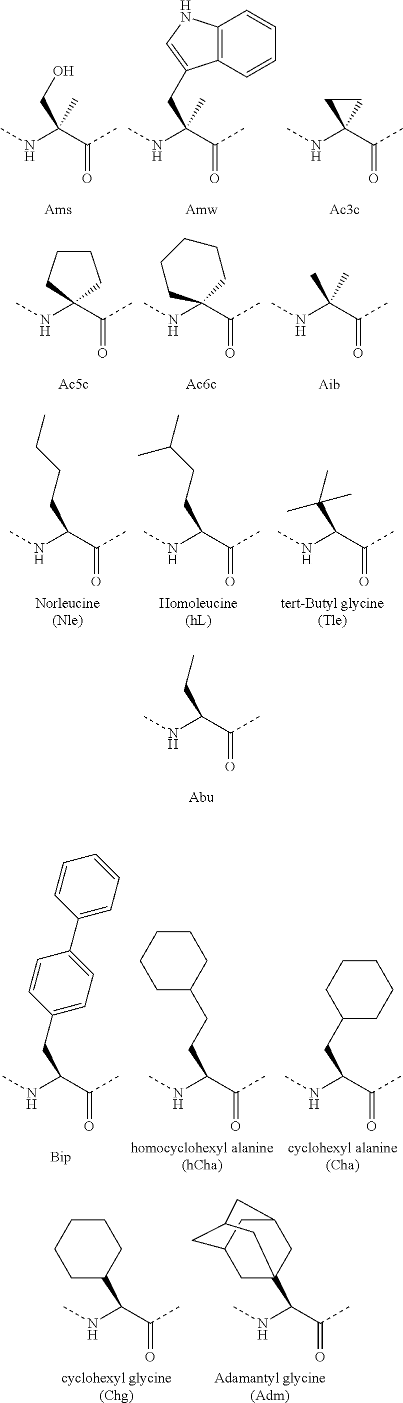

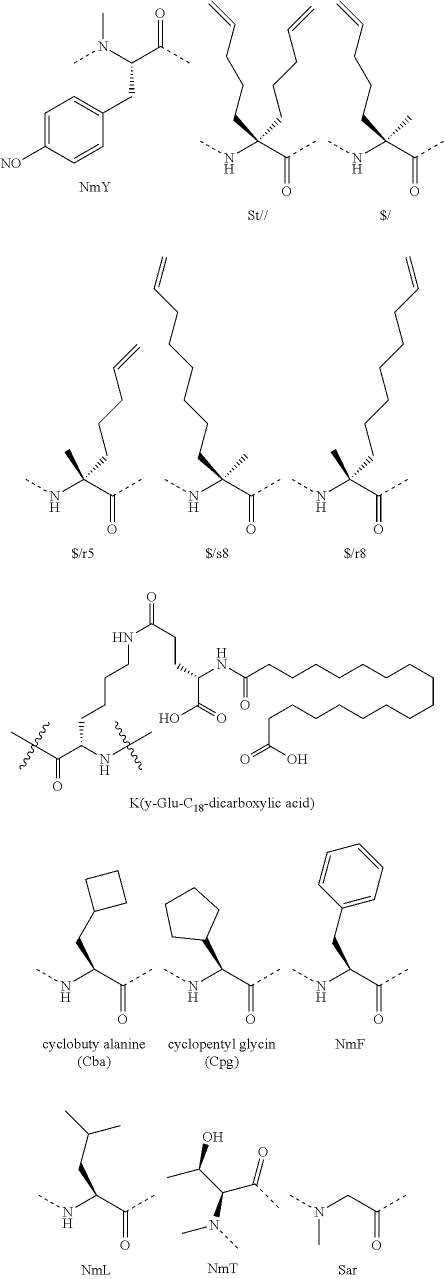

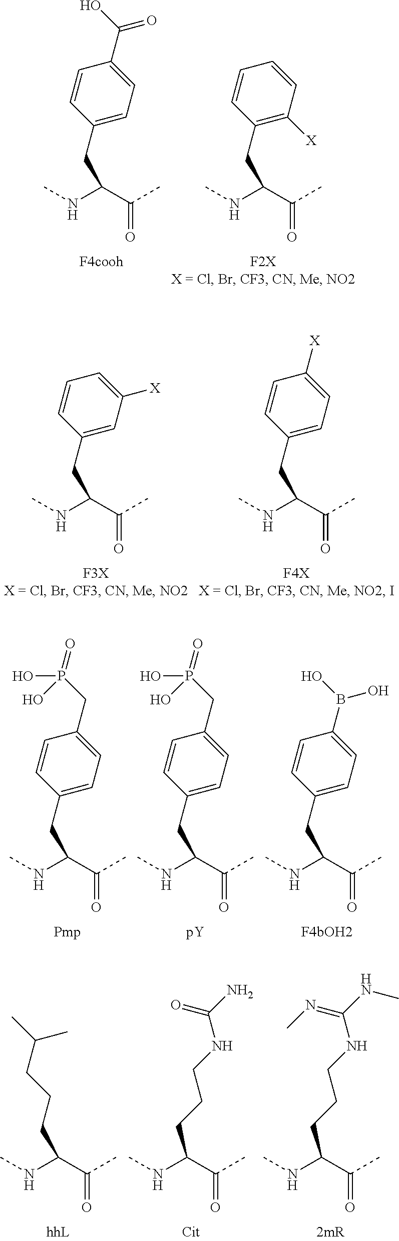

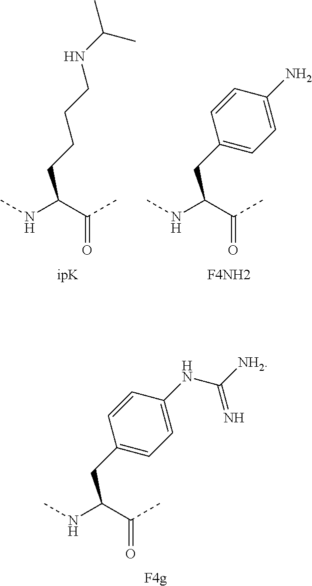

Amino acid analogs include β-amino acid analogs. Examples of β-amino acid analogs include, but are not limited to, the following: cyclic β-amino acid analogs; 3-alanine; (R)-3-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-3-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanine; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine.dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexylammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-3-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)₂-OH (symmetrical); Lys(ivDde)-OH; Lys(Me)₂-OH.HCl; Lys(Me3)-OH chloride; Nω-nitro-D-arginine; and Nω-nitro-L-arginine.

Amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs include analogs of tryptophan. Examples of amino acid analogs of tryptophan include, but are not limited to, the following: α-methyl-tryptophan; 3-(3-benzothienyl)-D-alanine; 3-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogs are racemic. In some embodiments, the D isomer of the amino acid analog is used. In some embodiments, the L isomer of the amino acid analog is used. In other embodiments, the amino acid analog comprises chiral centers that are in the R or S configuration. In still other embodiments, the amino group(s) of a α-amino acid analog is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a α-amino acid analog is protected, e.g., as its ester derivative. In some embodiments the salt of the amino acid analog is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions are substitutions based on isosteric considerations (e.g., norleucine for methionine) or other properties (e.g., 2-thienylalanine for phenylalanine, or 6-Cl-tryptophan for tryptophan).

The term “capping group” refers to the chemical moiety occurring at either the carboxy or amino terminus of the polypeptide chain of the subject peptidomimetic macrocycle. The capping group of a carboxy terminus includes an unmodified carboxylic acid (i.e. —COOH) or a carboxylic acid with a substituent. For example, the carboxy terminus can be substituted with an amino group to yield a carboxamide at the C-terminus. In some embodiments, the carboxy terminus can comprise a ghrelin agonist, such as those listed in Table 3. For example, the carboxy terminus can comprise hexarelin (L-Histidyl-2-methyl-D-tryptophyl-L-alanyl-L-tryptophyl-D-phenylalanyl-L-lysinamide. In some embodiments, the carboxy terminus can comprise a PEG. Various substituents include but are not limited to primary, secondary, and tertiary amines, including pegylated secondary amines. Representative secondary amine capping groups for the C-terminus include:

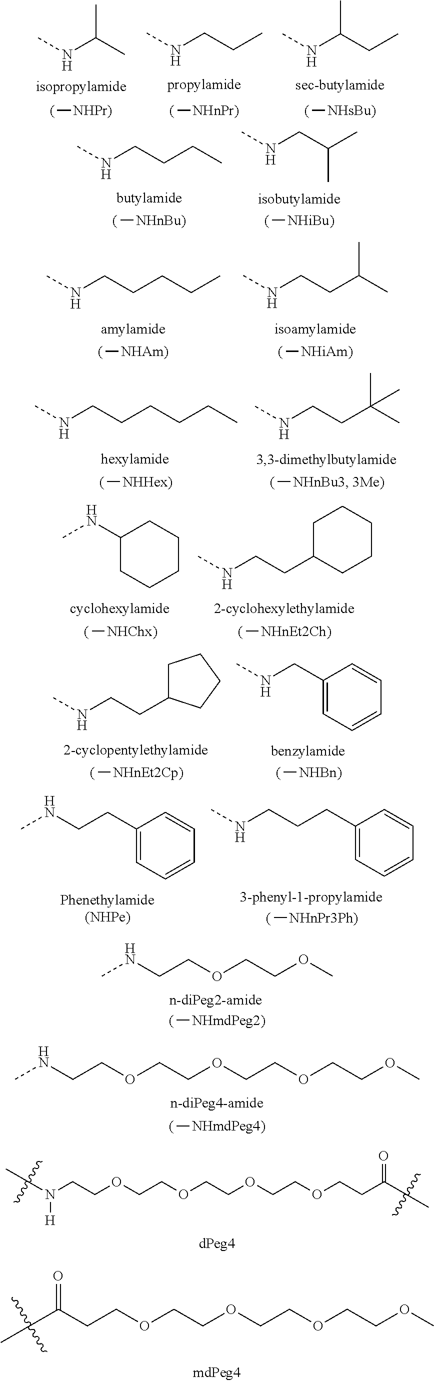

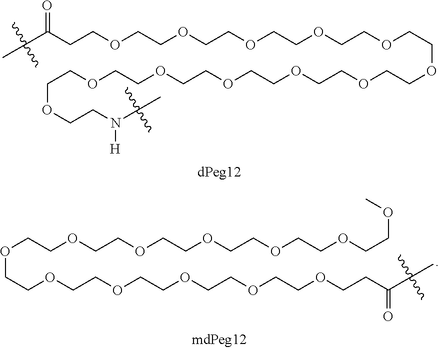 [SMILES: *NCCOCCOCCOCCOCCOCCOCCOCCOCCOCCOCCOCCOCCC(*)=O ; *C(=O)CCOCCOCCOCCOCCOCCOCCOCCOCCOCCOCCOCCOC]

The capping group of an amino terminus includes an unmodified amine (i.e. —NH₂) or an amine with a substituent. For example, the amino terminus can be substituted with an acyl group to yield a carboxamide at the N-terminus. Various substituents include but are not limited to substituted acyl groups, including C₁-C₆ carbonyls, C₇-C₃₀ carbonyls, and pegylated carbamates. Representative capping groups for the N-terminus include, but are not limited to, 4-FBzl (4-fluoro-benzyl) and the following:

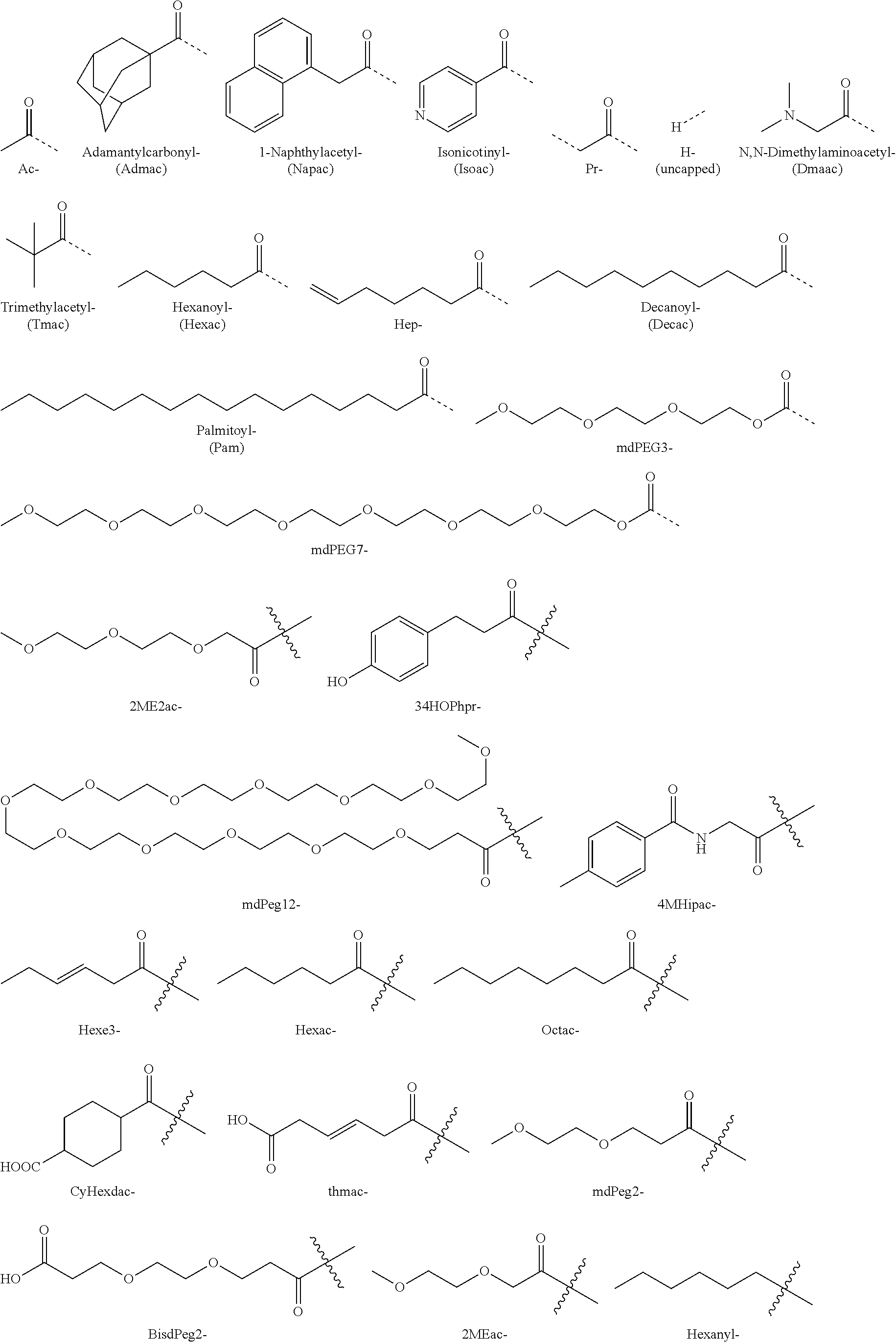

The term “member” as used as herein in conjunction with macrocycles or macrocycle-forming linkers refers to the atoms that form or can form the macrocycle, and excludes subsistent or side chain atoms. By analogy, cyclodecane 1,2-difluoro-decan and 1,3-dimenthyl cyclodecane are all consider ten-membered maracrocycles as the hydrogen or fluoro substituents or methyl side chains do not participate in forming the macrocycle.

The symbol “ [symbol] ” when used as part of a molecular structure refers to a dingle bond or a trans or cis double bond.

The term “amino acid side chain” refers to a moiety attached to the α-carbon (or another backbone atom) in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that occur in nature (e.g., an amino acid metabolite) or those that are made synthetically (e.g., an α,αdi-substituted amino acid).

The term “α,αdi-substituted amino acid” refers to a molecule or moiety containing both an amino group and a carboxyl group bound to a carbon (the α-carbon) that is attached to two natural or non-natural amino acid side chains.

The term “polypeptide” encompasses two or more naturally or non-naturally-occurring amino acids joined by a covalent bond (e.g., an amide bond). Polypeptides as described herein include full length proteins (e.g., fully processed proteins) as well as shorter amino acid sequences (e.g., fragments of naturally-occurring proteins or synthetic polypeptide fragments).

The term “first C-terminal amino acid” refers to the amino acid which is closest to the C-terminus. The term “second C-terminal amino acid” refers to the amino acid attached at the N-terminus of the first C-terminal amino acid.

The term “macrocyclization catalyst” or “macrocycle-forming catalyst” as used herein refers to any catalyst which can be used to prepare a peptidomimetic macrocycle by mediating the reaction between two reactive groups. Reactive groups can be, for example, an azide and alkyne, in which case macrocyclization catalysts include, without limitation, Cu catalysts such as catalysts which provide a reactive Cu(I) species, such as CuBr, Cul or CuOTf, as well as Cu(II) salts such as Cu(CO₂CH₃)₂, CuSO₄, and CuCl₂ that can be converted in situ to an active Cu(I) catalyst by the addition of a reducing agent such as ascorbic acid or sodium ascorbate. Macrocyclization catalysts can additionally include, for example, Ru catalysts known in the art such as Cp*RuCl(PPh₃)₂, [Cp*RuCl]₄ or other Ru catalysts which can provide a reactive Ru(II) species. In other cases, the reactive groups are terminal olefins. In such embodiments, the macrocyclization catalysts or macrocycle-forming catalysts are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. In other examples, catalysts have W or Mo centers. Various catalysts are disclosed in Grubbs et al., “Ring Closing Metathesis and Related Processes in Organic Synthesis” Acc. Chem. Res. 1995, 28, 446-452, U.S. Pat. No. 5,811,515; U.S. Pat. No. 7,932,397; U.S. Application No. 2011/0065915; U.S. Application No. 2011/0245477; Yu et al., “Synthesis of Macrocyclic Natural Products by Catalyst-Controlled Stereoselective Ring-Closing Metathesis,” Nature 2011, 479, 88; and Peryshkov et al., “Z-Selective Olefin Metathesis Reactions Promoted by Tungsten Oxo Alkylidene Complexes,” J. Am. Chem. Soc. 2011, 133, 20754. In yet other cases, the reactive groups are thiol groups. In such embodiments, the macrocyclization catalyst is, for example, a linker functionalized with two thiol-reactive groups such as halogen groups.

The term “halo” or “halogen” refers to fluorine, chlorine, bromine or iodine or a radical thereof.

The term “alkyl” refers to a hydrocarbon chain that is a straight chain or branched chain, containing the indicated number of carbon atoms. For example, C₁-C₁₀ indicates that the group has from 1 to 10 (inclusive) carbon atoms in it. In the absence of any numerical designation, “alkyl” is a chain (straight or branched) having 1 to 20 (inclusive) carbon atoms in it.

The term “alkylene” refers to a divalent alkyl (i.e., —R—).

The term “alkenyl” refers to a hydrocarbon chain that is a straight chain or branched chain having one or more carbon-carbon double bonds. The alkenyl moiety contains the indicated number of carbon atoms. For example, C₂-C₁₀ indicates that the group has from 2 to 10 (inclusive) carbon atoms in it. The term “lower alkenyl” refers to a C₂-C₆ alkenyl chain. In the absence of any numerical designation, “alkenyl” is a chain (straight or branched) having 2 to 20 (inclusive) carbon atoms in it.

The term “alkynyl” refers to a hydrocarbon chain that is a straight chain or branched chain having one or more carbon-carbon triple bonds. The alkynyl moiety contains the indicated number of carbon atoms. For example, C₂-C₁₀ indicates that the group has from 2 to 10 (inclusive) carbon atoms in it. The term “lower alkynyl” refers to a C₂-C₆ alkynyl chain. In the absence of any numerical designation, “alkynyl” is a chain (straight or branched) having 2 to 20 (inclusive) carbon atoms in it.

The term “aryl” refers to a monocyclic or bicyclic aromatic ring system wherein 0, 1, 2, 3, 4, or more atoms of each ring are substituted by a substituent. Exemplary aryls include 6-carbon monocyclic or 10-carbon bicyclic aromatic ring systems. Examples of aryl groups include phenyl, naphthyl and the like. The term “arylalkoxy” refers to an alkoxy substituted with aryl.

“Arylalkyl” refers to an aryl group, as defined above, wherein one of the aryl group's hydrogen atoms has been replaced with an alkyl group (e.g., a C₁-C₅ alkyl group) as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH₂-phenyl, 3-C(O)NH₂-phenyl, 4-C(O)NH₂-phenyl, 2-C(O)NH₂-pyridyl, 3-C(O)NH₂-pyridyl, and 4-C(O)NH₂-pyridyl,

“Alkylheterocycle” refers an alkyl group (e.g., a C₁-C₅ alkyl group), as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine, and —CH₂CH₂CH₂-imidazole.

“Alkylamido” refers to an alkyl group (e.g., a C₁-C₅ alkyl group), as defined above, wherein one of the alkyl group's hydrogen atoms has been replaced with a —C(O)NH₂ group. Representative examples of an alkylamido group include, but are not limited to, —CH₂—C(O)NH₂, —CH₂CH₂—C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃, —C(CH₃)₂CH₂C(O)NH₂, —CH₂—CH₂—NH—C(O)—CH₃, —CH₂—CH₂—NH—C(O)—CH₃—CH₃, and —CH₂—CH₂—NH—C(O)—CH═CH₂.

“Alkanol” refers to an alkyl group (e.g., a C₁-C₅ alkyl group), as defined above, wherein one of the alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂ CH₂CH₂OH, —CH₂CH(OH)CH₃, —CH₂CH(OH)CH₂CH₃, —CH(OH)CH₃ and —C(CH₃)₂CH₂OH.

“Alkylcarboxy” refers to an alkyl group (e.g., a C₁-C₅ alkyl group), as defined above, wherein one of the alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)₂CH₂COOH.

The term “cycloalkyl” as employed herein includes saturated and partially unsaturated cyclic hydrocarbon groups wherein the cycloalkyl group additionally is optionally substituted. For example a cycloalkyl can be saturated and partially unsaturated cyclic hydrocarbon groups having 3 to 12 carbons, 3 to 8 carbons, and or 3 to 6 carbons, Some cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic monocyclic, bicyclic, or tricyclic ring system having 1 or more heteroatoms. For example, a heteroaryl includes an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, the heteroatoms selected from O, N, or S-3 heteroatoms if monocyclic, 1-6 heteroatoms if bicyclic, or 1-9 heteroatoms if tricyclic, the heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, 4 or more atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heteroarylalkyl” or the term “heteroaralkyl” refers to an alkyl substituted with a heteroaryl.

The term “heteroarylalkoxy” refers to an alkoxy substituted with heteroaryl.

The term “heteroarylalkyl” or the term “heteroaralkyl” refers to an alkyl substituted with a heteroaryl.

The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, the heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

The term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In some embodiments, the compounds disclosed herein contain one or more asymmetric centers and thus occur as racemates and racemic mixtures, single enantiomers, individual diastereomers and diastereomeric mixtures. All such isomeric forms of these compounds are included unless expressly provided otherwise. In some embodiments, the compounds disclosed herein are also represented in multiple tautomeric forms, in such instances, the compounds include all tautomeric forms of the compounds described herein (e.g., if alkylation of a ring system results in alkylation at multiple sites, the invention includes all such reaction products). All such isomeric forms of such compounds are included unless expressly provided otherwise. All crystal forms of the compounds described herein are included unless expressly provided otherwise.

As used herein, the terms “increase” and “decrease” mean, respectively, to cause a statistically significantly (i.e., p<0.1) increase or decrease of at least 5%.

As used herein, the recitation of a numerical range for a variable is intended to convey that the variable is equal to any of the values within that range. Thus, for a variable which is inherently discrete, the variable is equal to any integer value within the numerical range, including the end-points of the range.

Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values ≧0 and ≦2 if the variable is inherently continuous.

As used herein, unless specifically indicated otherwise, the word “or” is used in the inclusive sense of “and/or” and not the exclusive sense of “either/or.”

The term “on average” represents the mean value derived from performing at least three independent replicates for each data point.

The term “biological activity” encompasses structural and functional properties of a macrocycle.

Biological activity is, for example, structural stability, alpha-helicity, affinity for a target, resistance to proteolytic degradation, in vivo stability, or any combination thereof.

The term “binding affinity” refers to the strength of a binding interaction, for example between a peptidomimetic macrocycle and a target. Binding affinity can be expressed, for example, as an equilibrium dissociation constant (“K_{D}”), which is expressed in units which are a measure of concentration (e.g., M, mM, μM, nM, etc.). Numerically, binding affinity and K_{D} values vary inversely, such that a lower binding affinity corresponds to a higher K_{D} value, while a higher binding affinity corresponds to a lower K_{D} value. Where high binding affinity is desirable, “improved” binding affinity refers to higher binding affinity i.e. lower K_{D} values.

The term “in vitro efficacy” refers to the extent to which a test compound, such as a peptidomimetic macrocycle, produces a beneficial result in an in vitro test system or assay. In vitro efficacy can be measured, for example, as an “IC₅₀” or “EC₅₀” value, which represents the concentration of the test compound which produces 50% of the maximal effect in the test system.

The term “ratio of in vitro efficacies” or “in vitro efficacy ratio” refers to the ratio of IC₅₀ or EC₅₀ values from a first assay (the numerator) versus a second assay (the denominator). Consequently, an improved in vitro efficacy ratio for Assay 1 versus Assay 2 refers to a lower value for the ratio expressed as IC₅₀ (Assay 1)/IC₅₀ (Assay 2) or alternatively as EC₅₀(Assay 1)/EC₅₀(Assay 2). This concept can also be characterized as “improved selectivity” in Assay 1 versus Assay 2, which can be due either to a decrease in the IC₅₀ or EC₅₀ value for Target 1 or an increase in the value for the IC₅₀ or EC₅₀ value for Target 2.

### Peptidomimetic Macrocycles

The details of one or more particular embodiments are set forth in the description below. In some embodiments, the peptide sequences are derived from a GHRH peptide. For example, the peptide sequences are derived from human GHRH (1-29) or human GHRH (1-44). A non-limiting exemplary list of suitable GHRH peptides for use is given in Table 1a, 1b, 2a, 2b and 2c below. The peptide sequences of GRF (1-32), tesamorelin (1-32), and sermorelin (GRF (1-29)), are depicted.

The full sequence of tesamorelin is

Hexe3-YADAIFTNSYRKVLGQLSARKLLQDIMSRQQGESNQERGARA
RL-NH₂

The full sequence of GRF (GRF (1-44)) is

H-YADAIFTNSYRKVLGQLSARKLLQDIMSRQQGESNQERGARARL-
NH₂.

TABLE 1

| SP# | | −1 | 0 | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|---|---|---|
| GRF | H— | | | Y | A | D | A | I | F | T | N | S | Y | R | K | V | L | G |
| 1 | Hexe3- | | | Y | A | D | A | I | F | T | N | S | Y | R | K | V | L | G |
| (Tesamorelin) | | | | | | | | | | | | | | | | | | |
| 2 | H— | | | Y | A | D | A | I | F | T | N | S | Y | R | K | V | L | G |
| (Sermorelin | | | | | | | | | | | | | | | | | | |
| (GRF 1-29)) | | | | | | | | | | | | | | | | | | |

| SP# | 16 | 17 | 18 | 19 | 20 | 21 | 22 | 23 | 24 | 25 | 26 | 27 | 28 | 29 | 30 | 31 | 32 | |
|---|---|---|---|---|---|---|---|---|---|---|---|---|---|---|---|---|---|---|
| GRF | Q | L | S | A | R | K | L | L | Q | D | I | M | S | R | Q | Q | G | —NH₂ |
| 1 | Q | L | S | A | R | K | L | L | Q | D | I | M | S | R | Q | Q | G | —NH₂ |
| (Tesamorelin) | | | | | | | | | | | | | | | | | | |
| 2 | Q | L | S | A | R | K | L | L | Q | D | I | M | S | R | | | | —NH₂ |
| (Sermorelin | | | | | | | | | | | | | | | | | | |
| (GRF 1-29)) | | | | | | | | | | | | | | | | | | |

TABLE 1a

| | | | | | | | | | | | | | | | | | | |
|---|---|---|---|---|---|---|---|---|---|---|---|---|---|---|---|---|---|---|
| 3 | H— | | | Y | a | D | $ | I | F | T | $ | S | Y | R | K | V | L | G |
| 4 | H— | | | Y | a | D | A | I | F | T | N | S | Y | R | $r8 | V | L | G |
| 9 | H— | | | NmY | A | D | A | I | F | T | $ | S | Y | R | $ | V | L | A |
| 7 | H— | | | NmY | A | D | $ | I | F | T | $ | S | Y | R | K | V | L | A |
| 11 | H— | | | NmY | A | D | A | I | F | T | A | S | Y | R | $ | V | L | A |
| 24 | H— | | | NmY | A | D | A | $r8 | F | T | A | S | Y | R | $ | V | L | A |
| 25 | H— | | | NmY | A | D | A | I | F | T | $r8 | S | Y | R | K | V | L | $ |
| 27 | H— | | | NmY | A | D | A | I | F | T | A | S | Y | R | K | V | L | $r8 |
| 18 | H— | | | NmY | A | D | A | I | F | T | A | S | Y | R | K | V | L | A |
| 30 | H— | | | NmY | A | D | A | I | F | T | A | S | Y | R | K | V | L | A |
| 32 | H— | | | NmY | A | D | A | I | F | T | A | S | Y | R | K | V | L | A |
| 33 | H— | | | NmY | A | D | A | I | F | T | A | S | Y | R | K | V | L | A |
| 56 | H— | P | P | Y | a | D | A | I | F | T | A | S | Y | R | $r8 | V | L | A |
| 57 | H— | | | NmY | A | D | A | I | F | T | A | S | Y | R | $r8 | V | L | A |
| 58 | H— | | | NmY | A | D | A | I | F | T | A | S | Y | R | $r8 | V | L | A |
| 59 | H— | | | NmY | A | D | A | I | F | T | A | S | Y | R | $r8 | V | L | A |
| 60 | H— | | | NmY | A | D | A | I | F | T | A | S | Y | R | $r8 | V | L | A |
| 61 | H— | | | NmY | A | D | A | I | F | T | A | S | Y | R | $r8 | V | L | A |
| 62 | H— | | | NmY | A | D | A | I | F | T | A | S | Y | R | $r8 | V | L | A |
| 63 | H— | | | NmY | A | D | A | I | F | T | A | S | Y | R | $r8 | V | L | A |
| 64 | H— | | | NmY | A | D | A | I | F | T | A | S | Y | R | $r8 | V | L | A |
| 65 | H— | | | NmY | A | D | A | I | F | T | A | S | Y | R | $r8 | V | L | A |
| 66 | H— | | | NmY | A | D | A | I | F | T | Aib | S | Y | R | $r8 | V | L | A |
| 67 | H— | | | NmY | A | D | A | I | F | T | Aib | S | Y | R | $r8 | V | L | A |
| 68 | H— | | | NmY | A | D | A | I | F | T | A | S | Y | R | $r8 | V | L | A |

| | | | | | | | | | | | | | | | | | | |
|---|---|---|---|---|---|---|---|---|---|---|---|---|---|---|---|---|---|---|
| 3 | Q | L | S | A | R | $ | L | L | Q | $ | I | Nle | S | R | | | | —NH₂ |
| 4 | Q | L | S | $ | R | K | L | L | Q | D | I | Nle | S | R | | | | —NH₂ |
| 9 | Q | L | S | A | R | K | A | L | Q | D | I | Nle | S | R | | | | —NH₂ |
| 7 | Q | L | S | A | R | K | A | L | Q | D | I | Nle | S | R | | | | —NH₂ |
| 11 | $ | L | S | A | R | K | A | L | Q | D | I | Nle | S | R | | | | —NH₂ |
| 24 | Q | L | S | A | R | K | A | L | Q | D | I | Nle | S | R | | | | —NH₂ |
| 25 | Q | L | S | A | R | K | A | L | Q | D | I | Nle | S | R | | | | —NH₂ |
| 27 | Q | L | S | A | R | K | $ | L | Q | D | I | Nle | S | R | | | | —NH₂ |
| 18 | Q | L | S | A | R | $ | A | L | Q | $ | I | Nle | S | R | | | | —NH₂ |
| 30 | Q | L | $r8 | A | R | K | A | L | Q | $ | I | Nle | S | R | | | | —NH₂ |
| 32 | Q | L | S | A | R | $r8 | A | L | Q | D | I | Nle | $ | R | | | | —NH₂ |
| 33 | Q | L | S | A | R | K | $r8 | L | Q | D | I | Nle | S | $ | | | | —NH₂ |
| 56 | Q | L | S | $ | R | K | A | L | Q | D | I | Nle | S | R | | | | —NH₂ |
| 57 | Q | L | S | $ | R | K | A | L | Q | D | I | Nle | S | R | | | | —NH₂ |
| 58 | A | L | S | $ | R | K | A | L | Q | D | I | Nle | S | R | | | | —NH₂ |
| 59 | Q | L | S | $ | R | K | A | L | Q | D | I | Nle | S | R | E | E | E | —NH₂ |
| 60 | Q | L | S | $ | R | K | A | L | Q | D | I | Nle | S | | | | | —NH₂ |
| 61 | Q | L | S | $ | R | K | A | L | Q | A | I | Nle | S | R | | | | —NH₂ |
| 62 | Q | L | S | $ | R | K | A | L | Q | D | A | Nle | S | R | | | | —NH₂ |
| 63 | Q | L | S | $ | R | K | A | L | A | D | I | Nle | S | R | | | | —NH₂ |
| 64 | Q | L | S | $ | R | K | A | L | Q | D | I | A | S | R | | | | —NH₂ |
| 65 | Q | L | S | $ | R | K | Aib | L | Q | D | I | Nle | S | R | | | | —NH₂ |
| 66 | Q | L | S | $ | R | K | Aib | L | Q | D | I | Nle | S | R | | | | —NH₂ |
| 67 | Q | L | S | $ | R | K | A | L | Aib | D | I | Nle | S | R | | | | —NH₂ |
| 68 | Q | L | S | $ | R | K | A | L | Q | D | I | Hse(Me) | S | R | | | | —NH₂ |

TABLE 1b

| SP# | | −1 | 0 | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|---|---|---|
| 5 | H— | | | Y | A | D | A | I | F | T | N | S | Y | R | K | V | L | G |
| 6 | H— | | $ | Y | A | D | $ | I | F | T | A | S | Y | R | K | V | L | A |
| 8 | H— | | | NmY | A | D | A | $ | F | T | A | $ | Y | R | K | V | L | A |
| 10 | H— | | | NmY | A | D | A | I | F | T | A | $ | Y | R | K | $ | L | A |
| 13 | H— | | | NmY | A | D | A | I | F | T | A | S | Y | R | K | V | $ | A |
| 14 | H— | | | NmY | A | D | A | I | F | T | A | S | Y | R | K | V | L | $ |
| 15 | H— | | | NmY | A | D | A | I | F | T | A | S | Y | R | K | V | L | A |
| 16 | H— | | | NmY | A | D | A | I | F | T | A | S | Y | R | K | V | L | A |
| 17 | H— | | | NmY | A | D | A | I | F | T | A | S | Y | R | K | V | L | A |
| 19 | H— | | | NmY | A | D | A | I | F | T | A | S | Y | R | K | V | L | A |
| 20 | H— | | | NmY | A | D | A | I | F | T | A | S | Y | R | K | V | L | A |
| 21 | H— | | | NmY | A | D | A | I | F | T | A | S | Y | R | K | V | L | A |
| 22 | H— | | | NmY | A | D | A | I | F | T | A | S | Y | R | K | V | L | A |
| 23 | H— | | | NmY | $r8 | D | A | I | F | T | A | $ | Y | R | K | V | L | A |
| 26 | H— | | | NmY | A | D | A | I | F | T | A | S | Y | R | K | V | $r8 | A |
| 28 | H— | | | NmY | A | D | A | I | F | T | A | S | Y | R | K | V | L | A |
| 29 | H— | | | NmY | A | D | A | I | F | T | A | S | Y | R | K | V | L | A |
| 31 | H— | | | NmY | A | D | A | I | F | T | A | S | Y | R | K | V | L | A |
| 34 | H— | | | NmY | A | D | A | I | F | T | N | S | Y | R | K | V | L | A |
| 35 | H— | | | NmY | A | D | A | I | F | T | A | S | Y | R | K | V | L | A |
| 36 | H— | | | Y | A | D | A | I | F | T | A | S | Y | R | K | V | L | A |
| 37 | H— | | | NipY | A | D | A | I | F | T | A | S | Y | R | K | V | L | A |
| 38 | 34HOPhpr- | | | | A | D | A | I | F | T | A | S | Y | R | K | V | L | A |
| 39 | Hexac- | | | Y | A | D | A | I | F | T | A | S | Y | R | K | V | L | A |
| 40 | Ac— | | | Y | A | D | A | I | F | T | A | S | Y | R | K | V | L | A |
| 41 | H— | | | NmY | A | D | A | I | F | T | Aib | S | Y | R | K | V | L | A |
| 42 | H— | | | NmY | A | D | A | I | F | T | T | S | Y | R | K | V | L | A |
| 43 | H— | | | NmY | A | D | A | I | F | T | Q | S | Y | R | K | V | L | A |
| 44 | H— | | | NmY | A | D | A | I | F | T | A | S | Y | R | K | V | L | Abu |
| 45 | H— | | | NmY | A | D | A | I | F | T | A | S | Y | R | K | V | L | A |
| 46 | H— | | | Y | a | D | A | I | F | T | A | S | Y | R | K | V | L | A |
| 47 | H— | | | NmY | A | D | A | I | F | T | A | S | Y | Cit | K | V | L | A |
| 48 | H— | | | NmY | A | D | A | I | F | T | A | S | Y | R | K | V | L | A |
| 49 | H— | | | NmY | A | D | A | I | F | T | A | S | Y | R | K | V | L | A |
| 50 | H— | | | NmY | A | D | A | I | F | T | A | S | Y | R | ipK | V | L | A |
| 51 | H— | | | NmY | A | D | A | I | F | T | A | S | Y | R | K | V | L | A |
| 52 | H— | | | NmY | A | D | A | I | F | T | Aib | S | Y | R | K | V | L | A |
| 53 | H— | | | NmY | A | D | A | I | F | T | Aib | S | Y | R | K | V | L | A |
| 54 | H— | | | NmY | A | D | A | I | F | T | A | S | Y | R | K | V | L | A |
| 55 | H— | | | NmY | A | D | A | I | F | T | Aib | S | Y | R | K | V | L | A |

| SP# | 16 | 17 | 18 | 19 | 20 | 21 | 22 | 23 | 24 | 25 | 26 | 27 | 28 | 29 | 30 | 31 | 32 | |
|---|---|---|---|---|---|---|---|---|---|---|---|---|---|---|---|---|---|---|
| 5 | Q | L | S | A | R | K | L | L | Q | D | I | Nle | S | R | | | | —NH₂ |
| 6 | Q | L | S | A | R | K | A | L | Q | D | I | Nle | S | R | | | | —NH₂ |
| 8 | Q | L | S | A | R | K | A | L | Q | D | I | Nle | S | R | | | | —NH₂ |
| 10 | Q | L | S | A | R | K | A | L | Q | D | I | Nle | S | R | | | | —NH₂ |
| 13 | Q | L | $ | A | R | K | A | L | Q | D | I | Nle | S | R | | | | —NH₂ |
| 14 | Q | L | S | $ | R | K | A | L | Q | D | I | Nle | S | R | | | | —NH₂ |
| 15 | Q | $ | S | A | R | $ | A | L | Q | D | I | Nle | S | R | | | | —NH₂ |
| 16 | Q | L | $ | A | R | K | $ | L | Q | D | I | Nle | S | R | | | | —NH₂ |
| 17 | Q | L | S | $ | R | K | A | $ | Q | D | I | Nle | S | R | | | | —NH₂ |
| 19 | Q | L | S | A | R | K | $ | L | Q | D | $ | Nle | S | R | | | | —NH₂ |
| 20 | Q | L | S | A | R | K | A | $ | Q | D | I | $ | S | R | | | | —NH₂ |
| 21 | Q | L | S | A | R | K | A | L | $ | D | I | Nle | $ | R | | | | —NH₂ |
| 22 | Q | L | S | A | R | K | A | L | Q | $ | I | Nle | S | $ | | | | —NH₂ |
| 23 | Q | L | S | A | R | K | A | L | Q | D | I | Nle | S | R | | | | —NH₂ |
| 26 | Q | L | S | A | R | $ | A | L | Q | D | I | Nle | S | R | | | | —NH₂ |
| 28 | $r8 | L | S | A | R | K | A | $ | Q | D | I | Nle | S | R | | | | —NH₂ |
| 29 | Q | $r8 | S | A | R | K | A | L | $ | D | I | Nle | S | R | | | | —NH₂ |
| 31 | Q | L | S | $r8 | R | K | A | L | Q | D | $ | Nle | S | R | | | | —NH₂ |
| 34 | Q | L | S | A | R | K | A | L | Q | D | I | Nle | S | R | | | | —NH₂ |
| 35 | Q | L | S | A | R | K | A | L | Q | D | I | Nle | S | R | | | | —NH₂ |
| 36 | Q | L | S | A | R | K | A | L | Q | D | I | Nle | S | R | | | | —NH₂ |
| 37 | Q | L | S | A | R | K | A | L | Q | D | I | Nle | S | R | | | | —NH₂ |
| 38 | Q | L | S | A | R | K | A | L | Q | D | I | Nle | S | R | | | | —NH₂ |
| 39 | Q | L | S | A | R | K | A | L | Q | D | I | Nle | S | R | | | | —NH₂ |
| 40 | Q | L | S | A | R | K | A | L | Q | D | I | Nle | S | R | | | | —NH₂ |
| 41 | Q | L | S | A | R | K | A | L | Q | D | I | Nle | S | R | | | | —NH₂ |
| 42 | Q | L | S | A | R | K | A | L | Q | D | I | Nle | S | R | | | | —NH₂ |
| 43 | Q | L | S | A | R | K | A | L | Q | D | I | Nle | S | R | | | | —NH₂ |
| 44 | Q | L | S | A | R | K | A | L | Q | D | I | Nle | S | R | | | | —NH₂ |
| 45 | Q | L | S | A | R | K | A | L | Q | D | I | Nle | D | R | | | | —NH₂ |
| 46 | Q | L | S | A | R | K | A | L | Q | D | I | Nle | S | R | | | | —NH₂ |
| 47 | Q | L | S | A | R | K | A | L | Q | D | I | Nle | S | R | | | | —NH₂ |
| 48 | Q | L | S | A | Cit | K | A | L | Q | D | I | Nle | S | R | | | | —NH₂ |
| 49 | Q | L | S | A | R | K | A | L | Q | D | I | Nle | S | Cit | | | | —NH₂ |
| 50 | Q | L | S | A | R | K | A | L | Q | D | I | Nle | S | R | | | | —NH₂ |
| 51 | Q | L | S | A | R | ipK | A | L | Q | D | I | Nle | S | R | | | | —NH₂ |
| 52 | Q | L | S | A | R | K | Aib | L | Q | D | I | Nle | S | R | | | | —NH₂ |
| 53 | Q | L | S | A | R | K | A | L | Aib | D | I | Nle | S | R | | | | —NH₂ |
| 54 | Q | L | Aib | A | R | K | A | L | A | D | I | Nle | S | R | | | | —NH₂ |
| 55 | Q | L | Aib | A | R | K | A | L | Aib | D | I | Nle | S | R | | | | —NH₂ |

TABLE 2

| SP# | | −1 | 0 | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|---|---|---|
| GRF | H— | | | Y | A | D | A | I | F | T | N | S | Y | R | K | V | L | G |
| 1 | Hexe3- | | | Y | A | D | A | I | F | T | N | S | Y | R | K | V | L | G |
| (Tesamorelin) | | | | | | | | | | | | | | | | | | |
| 2 | H— | | | Y | A | D | A | I | F | T | N | S | Y | R | K | V | L | G |
| (Sermorelin | | | | | | | | | | | | | | | | | | |
| (GRF 1-29)) | | | | | | | | | | | | | | | | | | |

| SP# | 16 | 17 | 18 | 19 | 20 | 21 | 22 | 23 | 24 | 25 | 26 | 27 | 28 | 29 | 30 | 31 | 32 | |
|---|---|---|---|---|---|---|---|---|---|---|---|---|---|---|---|---|---|---|
| GRF | Q | L | S | A | R | K | L | L | Q | D | I | M | S | R | | | | NH₂ |
| 1 | Q | L | S | A | R | K | L | L | Q | D | I | M | S | R | Q | Q | G | NH₂ |
| (Tesamorelin) | | | | | | | | | | | | | | | | | | |
| 2 | Q | L | S | A | R | K | L | L | Q | D | I | M | S | R | | | | NH₂ |
| (Sermorelin | | | | | | | | | | | | | | | | | | |
| (GRF 1-29)) | | | | | | | | | | | | | | | | | | |

TABLE 2a

| SP# | | −1 | 0 | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|---|---|---|
| 12 | H— | | | NmY | A | D | A | I | F | T | A | S | Y | R | K | $ | L | A |
| 69 | H— | | | NmY | a | D | A | I | F | T | Q | S | Y | R | K | $ | L | A |
| 70 | H— | | | NmY | a | D | A | I | F | T | Aib | S | Y | R | K | $ | L | A |
| 71 | H— | | | NmY | I | D | A | I | F | T | Q | S | Y | R | K | $ | L | A |
| 72 | H— | | | NmY | a | P | A | I | F | T | Q | S | Y | R | K | $ | L | A |
| 73 | H— | | | F₄COOH | a | D | A | I | F | T | Q | S | Y | R | K | $ | L | A |
| 74 | H— | | | F₄NH₂ | a | D | A | I | F | T | Q | S | Y | R | K | $ | L | A |
| 75 | H— | | P | Y | a | D | A | I | F | T | Q | S | Y | R | K | $ | L | A |
| 76 | H— | | K(γ-Glu-C₁₈— | Y | a | D | A | I | F | T | Q | S | Y | R | K | $ | L | A |
| | | | dicarboxylic acid) | | | | | | | | | | | | | | | |
| 77 | 4MHipac- | | | | A | D | A | I | F | T | Q | S | Y | R | K | $ | L | A |
| 78 | Hexe3- | | | Y | A | D | A | I | F | T | Q | S | Y | R | K | $ | L | A |
| 79 | Hexe3- | | | Y | a | D | A | I | F | T | Q | S | Y | R | K | $ | L | A |
| 80 | Hexac- | | | Y | a | D | A | I | F | T | Q | S | Y | R | K | $ | L | A |
| 81 | Octac- | | | Y | a | D | A | I | F | T | Q | S | Y | R | K | $ | L | A |
| 82 | mdPeg2- | | | Y | a | D | A | I | F | T | Q | S | Y | R | K | $ | L | A |
| 83 | mdPeg12- | | | Y | a | D | A | I | F | T | Q | S | Y | R | K | $ | L | A |
| 84 | 2MEac- | | | Y | a | D | A | I | F | T | Q | S | Y | R | K | $ | L | A |
| 85 | 2ME2ac- | | | Y | a | D | A | I | F | T | Q | S | Y | R | K | $ | L | A |
| 86 | BisdPeg2- | | | Y | a | D | A | I | F | T | Q | S | Y | R | K | $ | L | A |
| 87 | CyHexdac- | | | Y | a | D | A | I | F | T | Q | S | Y | R | K | $ | L | A |
| 88 | thmac- | | | Y | a | D | A | I | F | T | Q | S | Y | R | K | $ | L | A |
| 89 | ^{A}Hexanyl- | | | Y | a | D | A | I | F | T | Q | S | Y | R | K | $ | L | A |
| 90 | H— | | | NmY | a | D | A | I | F | T | A | S | Y | R | K | $ | L | A |
| 91 | H— | | | NmY | a | D | A | I | F | T | Q | S | Y | R | K | $ | L | A |
| 92 | H— | | | NmY | a | D | A | I | F | T | Q | S | Y | R | K | $ | L | A |
| 93 | H— | | | NmY | a | D | A | I | F | T | Q | S | Y | R | K | $ | L | A |
| 94 | H— | | | NmY | a | D | A | I | F | T | Q | S | Y | R | K | $ | L | A |
| 95 | H— | | | NmY | a | D | A | I | F | T | Q | S | Y | R | K | $5a5 | L | A |
| 96 | H— | | | NmY | a | D | A | I | F | T | Q | S | Y | R | K | $4a5 | L | A |
| 97 | H— | | | NmY | a | D | A | I | F | T | Q | S | Y | R | K | $5n3 | L | A |
| 98 | H— | | | NmY | a | D | A | I | F | T | Q | S | Y | R | K | $4n3 | L | A |
| 99 | H— | | | NmY | a | D | A | I | F | T | Q | S | Y | R | K | bK | L | A |
| 100 | H— | | | NmY | a | D | A | I | F | T | Q | S | Y | R | K | dialkyne | L | A |
| 101 | H— | | | NmY | a | D | A | I | F | T | Q | S | Y | R | K | $s6 | L | A |
| 102 | H— | | | NmY | a | D | A | I | F | T | Q | S | Y | R | K | $ | L | A |
| 103 | H— | | | NmY | a | D | A | I | F | T | Q | S | Y | R | K | $s6 | L | A |
| 104 | H— | | | Y | a | D | A | I | F | T | Q | S | Y | R | K | $ | L | A |
| 105 | H— | | | Y | a | D | A | I | F | T | Q | S | Y | R | K | $ | L | A |
| 106 | H— | | | Y | a | D | A | I | F | T | Q | S | Y | R | K | $ | L | A |
| 107 | H— | | | Y | a | D | A | I | F | T | Q | S | Y | R | K | $ | L | A |
| 108 | H— | | | Y | a | D | A | I | F | T | Q | S | Y | R | K | $ | L | A |
| 121 | H— | | | NmY | I | D | A | I | F | T | A | S | Y | R | K | $ | L | A |
| 122 | H— | | | NmY | a | P | A | I | F | T | A | S | Y | R | K | $ | L | A |
| 123 | H— | | | F₄COOH | a | D | A | I | F | T | A | S | Y | R | K | $ | L | A |
| 124 | H— | | | F₄NH₂ | a | D | A | I | F | T | A | S | Y | R | K | $ | L | A |
| 125 | H— | | P | Y | a | D | A | I | F | T | A | S | Y | R | K | $ | L | A |
| 126 | H— | | K(γ-Glu-C₁₈— | Y | a | D | A | I | F | T | A | S | Y | R | K | $ | L | A |
| | | | dicarboxylic acid) | | | | | | | | | | | | | | | |
| 127 | 4MHipac- | | | | A | D | A | I | F | T | A | S | Y | R | K | $ | L | A |
| 128 | Hexe3- | | | Y | A | D | A | I | F | T | A | S | Y | R | K | $ | L | A |
| 129 | Hexe3- | | | Y | a | D | A | I | F | T | A | S | Y | R | K | $ | L | A |
| 130 | Hexac- | | | Y | a | D | A | I | F | T | A | S | Y | R | K | $ | L | A |
| 131 | Octac- | | | Y | a | D | A | I | F | T | A | S | Y | R | K | $ | L | A |
| 132 | mdPeg2- | | | Y | a | D | A | I | F | T | A | S | Y | R | K | $ | L | A |
| 133 | mdPeg12- | | | Y | a | D | A | I | F | T | A | S | Y | R | K | $ | L | A |
| 134 | 2MEac- | | | Y | a | D | A | I | F | T | A | S | Y | R | K | $ | L | A |
| 135 | 2ME2ac- | | | Y | a | D | A | I | F | T | A | S | Y | R | K | $ | L | A |
| 136 | BisdPeg2- | | | Y | a | D | A | I | F | T | A | S | Y | R | K | $ | L | A |
| 137 | CyHexdac- | | | Y | a | D | A | I | F | T | A | S | Y | R | K | $ | L | A |
| 138 | thmac- | | | Y | a | D | A | I | F | T | A | S | Y | R | K | $ | L | A |
| 139 | ^{A}Hexanyl- | | | Y | a | D | A | I | F | T | A | S | Y | R | K | $ | L | A |

| SP# | 16 | 17 | 18 | 19 | 20 | 21 | 22 | 23 | 24 | 25 | 26 | 27 | 28 | 29 | 30 | 31 | 32 | |
|---|---|---|---|---|---|---|---|---|---|---|---|---|---|---|---|---|---|---|
| 12 | Q | $ | S | A | R | K | A | L | Q | D | I | Nle | S | R | | | | NH₂ |
| 69 | Q | $ | S | A | R | K | A | L | Q | D | I | Nle | S | R | | | | NH₂ |
| 70 | Q | $ | S | A | R | K | A | L | Q | D | I | Nle | S | R | | | | NH₂ |
| 71 | Q | $ | S | A | R | K | A | L | Q | D | I | Nle | S | R | | | | NH₂ |
| 72 | Q | $ | S | A | R | K | A | L | Q | D | I | Nle | S | R | | | | NH₂ |
| 73 | Q | $ | S | A | R | K | A | L | Q | D | I | Nle | S | R | | | | NH₂ |
| 74 | Q | $ | S | A | R | K | A | L | Q | D | I | Nle | S | R | | | | NH₂ |
| 75 | Q | $ | S | A | R | K | A | L | Q | D | I | Nle | S | R | | | | NH₂ |
| 76 | Q | $ | S | A | R | K | A | L | Q | D | I | Nle | S | R | | | | NH₂ |
| 77 | Q | $ | S | A | R | K | A | L | Q | D | I | Nle | S | R | | | | NH₂ |
| 78 | Q | $ | S | A | R | K | A | L | Q | D | I | Nle | S | R | | | | NH₂ |
| 79 | Q | $ | S | A | R | K | A | L | Q | D | I | Nle | S | R | | | | NH₂ |
| 80 | Q | $ | S | A | R | K | A | L | Q | D | I | Nle | S | R | | | | NH₂ |
| 81 | Q | $ | S | A | R | K | A | L | Q | D | I | Nle | S | R | | | | NH₂ |
| 82 | Q | $ | S | A | R | K | A | L | Q | D | I | Nle | S | R | | | | NH₂ |
| 83 | Q | $ | S | A | R | K | A | L | Q | D | I | Nle | S | R | | | | NH₂ |
| 84 | Q | $ | S | A | R | K | A | L | Q | D | I | Nle | S | R | | | | NH₂ |
| 85 | Q | $ | S | A | R | K | A | L | Q | D | I | Nle | S | R | | | | NH₂ |
| 86 | Q | $ | S | A | R | K | A | L | Q | D | I | Nle | S | R | | | | NH₂ |
| 87 | Q | $ | S | A | R | K | A | L | Q | D | I | Nle | S | R | | | | NH₂ |
| 88 | Q | $ | S | A | R | K | A | L | Q | D | I | Nle | S | R | | | | NH₂ |
| 89 | Q | $ | S | A | R | K | A | L | Q | D | I | Nle | S | R | | | | NH₂ |
| 90 | Q | $ | S | A | R | K | A | L | Q | D | I | Nle | S | R | | | | NH₂ |
| 91 | A | $ | S | A | R | K | A | L | Q | D | I | Nle | S | R | | | | NH₂ |
| 92 | E | $ | S | A | R | K | A | L | Q | D | I | Nle | S | R | | | | NH₂ |
| 93 | Nle | $ | S | A | R | K | A | L | Q | D | I | Nle | S | R | | | | NH₂ |
| 94 | S | $ | S | A | R | K | A | L | Q | D | I | Nle | S | R | | | | NH₂ |
| 95 | Q | $5n3 | S | A | R | K | A | L | Q | D | I | Nle | S | R | | | | NH₂ |
| 96 | Q | $4n3 | S | A | R | K | A | L | Q | D | I | Nle | S | R | | | | NH₂ |
| 97 | Q | $5a5 | S | A | R | K | A | L | Q | D | I | Nle | S | R | | | | NH₂ |
| 98 | Q | $4a5 | S | A | R | K | A | L | Q | D | I | Nle | S | R | | | | NH₂ |
| 99 | Q | bE | S | A | R | K | A | L | Q | D | I | Nle | S | R | | | | NH₂ |
| 100 | Q | dialkyne | S | A | R | K | A | L | Q | D | I | Nle | S | R | | | | NH₂ |
| 101 | Q | $ | S | A | R | K | A | L | Q | D | I | Nle | S | R | | | | NH₂ |
| 102 | Q | $s6 | S | A | R | K | A | L | Q | D | I | Nle | S | R | | | | NH₂ |
| 103 | Q | $s6 | S | A | R | K | A | L | Q | D | I | Nle | S | R | | | | NH₂ |
| 104 | A | $ | S | A | R | K | A | L | Q | D | I | Nle | S | R | | | | NH₂ |
| 105 | E | $ | S | A | R | K | A | L | Q | D | I | Nle | S | R | | | | NH₂ |
| 106 | Nle | $ | S | A | R | K | A | L | Q | D | I | Nle | S | R | | | | NH₂ |
| 107 | S | $ | S | A | R | K | A | L | Q | D | I | Nle | S | R | | | | NH₂ |
| 108 | Q | $ | S | A | R | K | A | L | Q | D | I | Nle | S | R | | | | NH₂ |
| 121 | Q | $ | S | A | R | K | A | L | Q | D | I | Nle | S | R | | | | NH₂ |
| 122 | Q | $ | S | A | R | K | A | L | Q | D | I | Nle | S | R | | | | NH₂ |
| 123 | Q | $ | S | A | R | K | A | L | Q | D | I | Nle | S | R | | | | NH₂ |
| 124 | Q | $ | S | A | R | K | A | L | Q | D | I | Nle | S | R | | | | NH₂ |
| 125 | Q | $ | S | A | R | K | A | L | Q | D | I | Nle | S | R | | | | NH₂ |
| 126 | Q | $ | S | A | R | K | A | L | Q | D | I | Nle | S | R | | | | NH₂ |
| 127 | Q | $ | S | A | R | K | A | L | Q | D | I | Nle | S | R | | | | NH₂ |
| 128 | Q | $ | S | A | R | K | A | L | Q | D | I | Nle | S | R | | | | NH₂ |
| 129 | Q | $ | S | A | R | K | A | L | Q | D | I | Nle | S | R | | | | NH₂ |
| 130 | Q | $ | S | A | R | K | A | L | Q | D | I | Nle | S | R | | | | NH₂ |
| 131 | Q | $ | S | A | R | K | A | L | Q | D | I | Nle | S | R | | | | NH₂ |
| 132 | Q | $ | S | A | R | K | A | L | Q | D | I | Nle | S | R | | | | NH₂ |
| 133 | Q | $ | S | A | R | K | A | L | Q | D | I | Nle | S | R | | | | NH₂ |
| 134 | Q | $ | S | A | R | K | A | L | Q | D | I | Nle | S | R | | | | NH₂ |
| 135 | Q | $ | S | A | R | K | A | L | Q | D | I | Nle | S | R | | | | NH₂ |
| 136 | Q | $ | S | A | R | K | A | L | Q | D | I | Nle | S | R | | | | NH₂ |
| 137 | Q | $ | S | A | R | K | A | L | Q | D | I | Nle | S | R | | | | NH₂ |
| 138 | Q | $ | S | A | R | K | A | L | Q | D | I | Nle | S | R | | | | NH₂ |
| 139 | Q | $ | S | A | R | K | A | L | Q | D | I | Nle | S | R | | | | NH₂ |

TABLE 2b

| | | | | | | | | | | | | | | | | |
|---|---|---|---|---|---|---|---|---|---|---|---|---|---|---|---|---|
| 109 | 2ME2ac- | Y | a | D | A | I | F | T | Q | S | Y | R | K | $ | L | A |
| 110 | 2ME2ac- | Y | a | D | A | I | F | T | Q | S | Y | R | K | $ | L | A |
| 111 | 2ME2ac- | Y | a | D | A | I | F | T | Q | S | Y | R | K | $ | L | A |
| 112 | 2ME2ac- | Y | a | D | A | I | F | T | Q | S | Y | R | K | $ | L | A |
| 113 | H— | Y | a | D | A | I | F | T | Q | S | Y | R | K | $ | L | A |
| 114 | H— | Y | a | D | A | I | F | T | Q | S | Y | R | K | $ | L | A |
| 115 | H— | Y | a | D | A | I | F | T | Q | S | Y | R | K | $ | L | A |
| 116 | H— | Y | a | D | A | I | F | T | Q | S | Y | R | K | $ | L | A |

| | | | | | | | | | | | | | | | | | | |
|---|---|---|---|---|---|---|---|---|---|---|---|---|---|---|---|---|---|---|
| 109 | Q | $ | S | A | R | K | A | L | Q | D | I | Nle | S | R | ^{B}K(dPeg4-dPeg4- | | | NH₂ |
| | | | | | | | | | | | | | | | mdPeg4) | | | |
| 110 | Q | $ | S | A | R | K | A | L | Q | D | I | Nle | S | R | dPeg4 | dPeg4 | dPeg4 | NH₂ |
| 111 | Q | $ | S | A | R | K | A | L | Q | D | I | Nle | S | R | ^{B}K(mdPeg12) | | | NH₂ |
| 112 | Q | $ | S | A | R | K | A | L | Q | D | I | Nle | S | R | dPeg12 | | | NH₂ |
| 113 | Q | $ | S | A | R | K | A | L | Q | D | I | Nle | S | R | ^{B}K(dPeg4-dPeg4- | | | NH₂ |
| | | | | | | | | | | | | | | | mdPeg4) | | | |
| 114 | Q | $ | S | A | R | K | A | L | Q | D | I | Nle | S | R | dPeg4 | dPeg4 | dPeg4 | NH₂ |
| 115 | Q | $ | S | A | R | K | A | L | Q | D | I | Nle | S | R | ^{B}K(mdPeg12) | | | NH₂ |
| 116 | Q | $ | S | A | R | K | A | L | Q | D | I | Nle | S | R | dPeg12 | | | NH₂ |

TABLE 2c

| SP# | | −1 | 0 | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|---|---|---|
| 117 | 2ME2ac- | | | Y | a | D | A | I | F | T | Q | S | Y | R | K | $ | L | A |
| 118 | 2ME2ac- | | | Y | a | D | A | I | F | T | Q | S | Y | R | K | $ | L | A |
| 119 | H— | | | Y | a | D | A | I | F | T | Q | S | Y | R | K | $ | L | A |
| 120 | H— | | | Y | a | D | A | I | F | T | Q | S | Y | R | K | $ | L | A |

| SP# | 16 | 17 | 18 | 19 | 20 | 21 | 22 | 23 | 24 | 25 | 26 | 27 | 28 | 29 | 30 | 31 | 32 | |
|---|---|---|---|---|---|---|---|---|---|---|---|---|---|---|---|---|---|---|
| 117 | Q | $ | S | A | R | K | A | L | Q | D | I | Nle | S | R | ^{B}K(dPeg4-dPeg4- | | | NH₂ |
| | | | | | | | | | | | | | | | dPeg4-hexarelin) | | | |
| 118 | Q | $ | S | A | R | K | A | L | Q | D | I | Nle | S | R | ^{B}K(dPeg4- | | | NH₂ |
| | | | | | | | | | | | | | | | hexarelin) | | | |
| 119 | Q | $ | S | A | R | K | A | L | Q | D | I | Nle | S | R | ^{B}K(dPeg4-dPeg4- | | | NH₂ |
| | | | | | | | | | | | | | | | dPeg4-hexarelin) | | | |
| 120 | Q | $ | S | A | R | K | A | L | Q | D | I | Nle | S | R | ^{B}K(dPeg4- | | | NH₂ |
| | | | | | | | | | | | | | | | hexarelin) | | | |

In the sequences shown above and elsewhere, the following abbreviations are used: “Nle” represents norleucine, “Aib” represents 2-aminoisobutyric acid, “Ac” represents acetyl, and “Pr” represents propionyl. Amino acids represented as “$” are alpha-Me S5-pentenyl-alanine olefin amino acids connected by an all-carbon crosslinker comprising one double bond. Amino acids represented as “$r5” are alpha-Me R5-pentenyl-alanine olefin amino acids connected by an all-carbon comprising one double bond. Amino acids represented as “$s8” are alpha-Me S8-octenyl-alanine olefin amino acids connected by an all-carbon crosslinker comprising one double bond. Amino acids represented as “$r8” are alpha-Me R8-octenyl-alanine olefin amino acids connected by an all-carbon crosslinker comprising one double bond. “Ahx” represents an aminocyclohexyl linker. The crosslinkers are linear all-carbon crosslinker comprising eight or eleven carbon atoms between the alpha carbons of each amino acid. Amino acids represented as “$/” are alpha-Me S5-pentenyl-alanine olefin amino acids that are not connected by any crosslinker. Amino acids represented as “$/r5” are alpha-Me R5-pentenyl-alanine olefin amino acids that are not connected by any crosslinker. Amino acids represented as “$/s8” are alpha-Me S8-octenyl-alanine olefin amino acids that are not connected by any crosslinker. Amino acids represented as “$/r8” are alpha-Me R8-octenyl-alanine olefin amino acids that are not connected by any crosslinker. Amino acids represented as “Amw” are alpha-Me tryptophan amino acids. Amino acids represented as “Aml” are alpha-Me leucine amino acids. Amino acids represented as “Amf” are alpha-Me phenylalanine amino acids. Amino acids represented as “2ff” are 2-fluoro-phenylalanine amino acids. Amino acids represented as “3ff” are 3-fluoro-phenylalanine amino acids. Amino acids represented as “St” are amino acids comprising two pentenyl-alanine olefin side chains, each of which is crosslinked to another amino acid as indicated. Amino acids represented as “St//” are amino acids comprising two pentenyl-alanine olefin side chains that are not crosslinked. Amino acids represented as “% St” are amino acids comprising two pentenyl-alanine olefin side chains, each of which is crosslinked to another amino acid as indicated via fully saturated hydrocarbon crosslinks. Amino acids represented as “Ba” are beta-alanine. The lower-case character “e” or “z” within the designation of a crosslinked amino acid (e.g., “$er8” or “$zr8”) represents the configuration of the double bond (E or Z, respectively). In other contexts, lower-case letters such as “a” or “f” represent D amino acids (e.g., D-alanine, or D-phenylalanine, respectively). Amino acids designated as “NmW” represent N-methyltryptophan. Amino acids designated as “NmY” represent N-methyltyrosine. Amino acids designated as “NmA” represent N-methylalanine. “Kbio” represents a biotin group attached to the side chain amino group of a lysine residue. Amino acids designated as “Sar” represent sarcosine. Amino acids designated as “Cha” represent cyclohexyl alanine. Amino acids designated as “Cpg” represent cyclopentyl glycine. Amino acids designated as “Chg” represent cyclohexyl glycine. Amino acids designated as “Cba” represent cyclobutyl alanine. Amino acids designated as “F₄I” represent 4-iodo phenylalanine. “7L” represents N15 isotopic leucine. Amino acids designated as “F₃Cl” represent 3-chloro phenylalanine. Amino acids designated as “F4cooh” represent 4-carboxy phenylalanine. Amino acids designated as “F₃4F₂” represent 3,4-difluoro phenylalanine. Amino acids designated as “6clW” represent 6-chloro tryptophan. Amino acids designated as “$rda6” represent alpha-Me R6-hexynyl-alanine alkynyl amino acids, crosslinked via a dialkyne bond to a second alkynyl amino acid. Amino acids designated as “$da5” represent alpha-Me S5-pentynyl-alanine alkynyl amino acids, wherein the alkyne forms one half of a dialkyne bond with a second alkynyl amino acid. Amino acids designated as “$ra9” represent alpha-Me R9-nonynyl-alanine alkynyl amino acids, crosslinked via an alkyne metathesis reaction with a second alkynyl amino acid. Amino acids designated as “$s6” represent alpha-Me S6-hexynyl-alanine alkynyl amino acids, crosslinked via an alkyne metathesis reaction with a second alkynyl amino acid. The designation “iso1” or “iso2” indicates that the peptidomimetic macrocycle is a single isomer. Amino acids designated as “Cit” represent citrulline.

A peptidomimetic macrocycle can include a drug, a toxin, a derivative of polyethylene glycol; a second polypeptide; a carbohydrate, etc. Where a polymer or other agent is linked to a peptidomimetic macrocycle, it can be desirable for the composition to be substantially homogeneous. The addition of polyethelene glycol (PEG) molecules can improve the pharmacokinetic and pharmacodynamic properties of the polypeptide. For example, PEGylation can reduce renal clearance and can result in a more stable plasma concentration. PEG is a water soluble polymer and can be represented as linked to the polypeptide as formula: X0-(CH₂CH₂0)ₙ—CH₂CH₂—Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the polypeptide. Y may also be a maleimide linkage to a thiol group (including but not limited to, the thiol group of cysteine). Other methods for linking PEG to a polypeptide, directly or indirectly, are known to those of ordinary skill in the art. The PEG can be linear or branched. Various forms of PEG including various functionalized derivatives are commercially available. In some embodiments, PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In some embodiments, a peptidomimetic macrocycle can be prepared based on solubility of the polypeptide, for example if the prepared peptidomimetic macrocycle is determined to be soluble based on visual examination of the turbidity of a solution of the polypeptide. In some embodiments, an aqueous solubility of the peptidomimetic macrocycle is determined by evaluating the turbidity of a solution comprising the peptidomimetic macrocycle. In some embodiments, a plasma solubility of the peptidomimetic macrocycle is determined by evaluating the turbidity of a solution comprising the peptidomimetic macrocycle.

In some embodiments, a peptidomimetic macrocycle comprises a ghrelin agonist. For example, a peptidomimetic macrocycle can be conjugated to a ghrelin agonist. In some embodiments, the peptidomimetic macrocycle comprises a ghrelin agonist, such as those listed in Table 3. In some embodiments, the peptidomimetic macrocycle comprises a ghrelin agonist, wherein the ghrelin agonist is conjugated to an amino acid such as Lys. In some embodiments, the conjugated Lys is conjugated to a PEG. In some embodiments, the conjugated Lys comprises Lys([PEG]ₙ), where n is 1, 2, 3, 4, 5, 6, 7, 8, 9, 10, or more. In some embodiments, the conjugated Lys is conjugated to a ghrelin agonist. In some embodiments, the conjugated Lys comprises Lys(dPeg₄-dPeg₄-mdPeg₄). In some embodiments, the conjugated Lys comprises Lys (mdPeg₁₂). In some embodiments, the conjugated Lys comprises Lys(dPeg₄-dPeg₄-dPeg₄-[ghrelin agonist]). In some embodiments, the conjugated Lys comprises Lys(dPeg₄-[ghrelin agonist]).

In some embodiments, the peptidomimetic macrocycle comprises PEG, wherein the PEG is optionally conjugated to an amino acid such as Lys. In some embodiments, the peptidomimetic macrocycle comprises a ghrelin agonist, such as a ghrelin agonist of Table 3, wherein the ghrelin agonist is optionally conjugated to an amino acid such as Lys. In some embodiments, the peptidomimetic macrocycle comprises a spacer (such as PEG), wherein the spacer is optionally conjugated to an amino acid such as Lys. In some embodiments, the peptidomimetic macrocycle comprises a ghrelin agonist, wherein the ghrelin agonist is optionally conjugated to an amino acid such as Lys. In some embodiments, the ghrelin agonist is selected from the group consisting of hexarelin, anamorelin, capromorelin, GHRP-6, ibutamoren, ipamorelin, macimorelin, pralmorelin, relamorelin and tabimorelin In some embodiments, the peptidomimetic macrocycle comprises a spacer and/or a Ghrelin agonist wherein the spacer and/or Ghrelin agonist is conjugated to a Lys, wherein the conjugated Lys is located at one or more of the following locations: 8, 9, 15, 16, 18, 19, 22, 24, 25, 26, 28 or 30; of amino acids 1-30 of Human Growth Hormone-Release Hormone (GHRH 1-32. In some embodiments, the peptidomimetic macrocycle comprises PEG, wherein the PEG is optionally conjugated to an amino acid such as Lys. In some embodiments, the peptidomimetic macrocycle comprises a ghrelin agonist selected from a ghrelin agonist of Table 3, wherein the ghrelin agonist is optionally conjugated to an amino acid. In some embodiments, the conjugated amino acid is conjugated to a spacer, such as PEG. In some embodiments, the conjugated amino acid is Lys. In some embodiments, the conjugated amino acid is Lys(dPeg₄-dPeg₄-dPeg₄-[Ghrelin agonist]). In some embodiments, the conjugated amino acid is Lys(dPeg₄-[Ghrelin agonist]). In some embodiments, the conjugated Lys is conjugated to a Ghrelin agonist, a spacer (such as a PEG), or both.

TABLE 3
Table 3 - Ghrelin agonists

| Name | IUPAC Name | Structure |
|---|---|---|
| Hexarelin | (2S)-6-amino-2-[[(2R)-2-[[(2S)- 2-[[(2S)-2-[[(2R)-2-[[(2S)-2- amino-3-(1H-imidazol-5- yl)propanoyl]amino]-3-(2- methyl-1H-indol-3- yl)propanoyl]amino]propanoyl] amino]-3-(1H-indol-3- yl)propanoyl]amino]-3- phenylpropanoyl]amino] hexanamide | 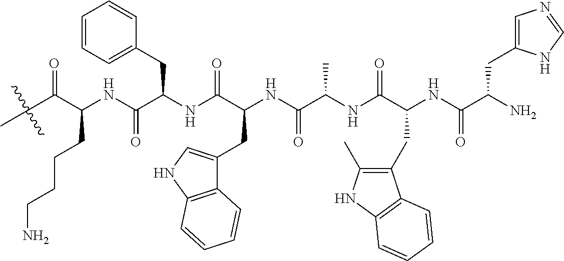 [SMILES: *C(=O)[C@H](CCCCN)NC(=O)[C@@H](Cc1ccccc1)NC(=O)[C@H](Cc1c[nH]c2ccccc12)NC(=O)[C@H](C)NC(=O)[C@@H](Cc1c(C)[nH]c2ccccc12)NC(=O)[C@@H](N)Cc1cnc[nH]1] |
| Anamorelin | 2-Amino-N-[(2R)-1-[(3R)-3- benzyl-3- [dimethylamino(methyl)carbamoyl] piperidin-1-yl]-3-(1H-indol-3- yl)-1-oxopropan-2-yl]-2- methylpropanamide | 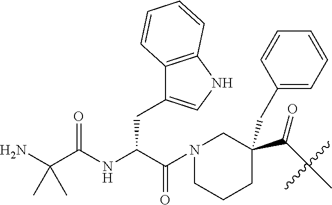 [SMILES: *C(=O)[C@@]1(Cc2ccccc2)CCCN(C(=O)[C@@H](Cc2c[nH]c3ccccc23)NC(=O)C(C)(C)N)C1] |
| Capromorelin | N-[(2R)-1-[(3aR)-2-methyl-3- oxo-3a-(phenylmethyl)-6,7- dihydro-4H-pyrazolo[4,3- c]pyridin-5-yl]-1-oxo-3- (phenylmethoxy)propan-2-yl]-2- amino-2-methylpropanamide | 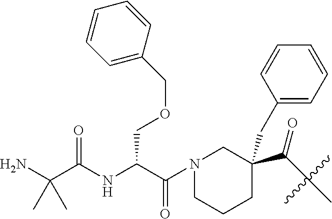 [SMILES: *C(=O)[C@@]1(Cc2ccccc2)CCCN(C(=O)[C@@H](COCc2ccccc2)NC(=O)C(C)(C)N)C1] |
| GHRP-6 | L-histidyl-D-tryptophyl-L-alanyl- L-tryptophyl-D-phenylalanyl-L- Lysinamide | 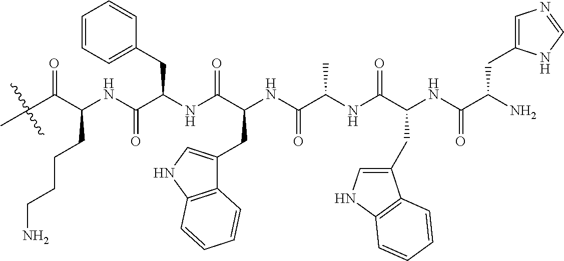 [SMILES: *C(=O)[C@H](CCCCN)NC(=O)[C@@H](Cc1ccccc1)NC(=O)[C@H](Cc1c[nH]c2ccccc12)NC(=O)[C@H](C)NC(=O)[C@@H](Cc1c[nH]c2ccccc12)NC(=O)[C@@H](N)Cc1cnc[nH]1] |
| Ibutamoren | (R)-1′-(2-methylalanyl-O-benzyl- D-seryl)-1-(methylsulfonyl)-1,2- dihydrospiro[indole-3,4′- piperidine] | 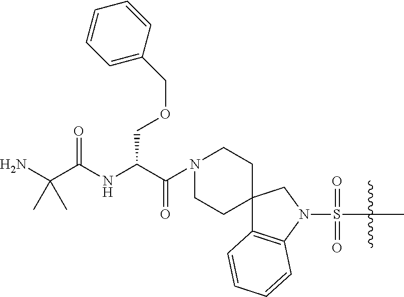 [SMILES: *S(=O)(=O)N1CC2(CCN(C(=O)[C@@H](COCc3ccccc3)NC(=O)C(C)(C)N)CC2)c2ccccc21] |
| Ipamorelin | (2S)-6-Amino-2-[[(2R)-2-[[(2R)- 2-[[(2S)-2-[(2-amino-2- methylpropanoyl)amino]-3-(4H- imidazol-4-yl )propanoyl)amino]- 3-naphthalen-2- ylpropanoyl]amino]-3- phenylpropanoyl]amino] hexanamide | 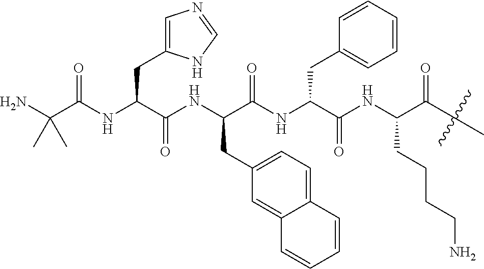 [SMILES: *C(=O)[C@H](CCCCN)NC(=O)[C@@H](Cc1ccccc1)NC(=O)[C@@H](Cc1ccc2ccccc2c1)NC(=O)[C@H](Cc1cnc[nH]1)NC(=O)C(C)(C)N] |
| Macimorelin | 2-Amino-N-[(2R)-1-[[(1R)-1- formamido-2-(1H-indol-3- yl)ethyl]amino]-3-1H-indol-3-yl)- 1-oxopropan-2-yl]-2- methylpropanamide | 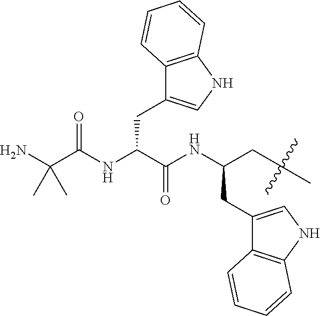 [SMILES: *C[C@@H](Cc1c[nH]c2ccccc12)NC(=O)[C@@H](Cc1c[nH]c2ccccc12)NC(=O)C(C)(C)N] |
| Pralmorelin | (2S)-6-Amino-2-[[(2S)-2-[[(2S)- 2-[[(2S)-2-[[(2R)-2-[[(2R)-2- aminopropanoyl]amino]-3- naphthalen-2- ylpropanoyl]amino]propanoyl] amino]-3-(1H-indol-3- yl)propanoyl]amino]-3- phenylpropanoyl]amino] hexanamide | 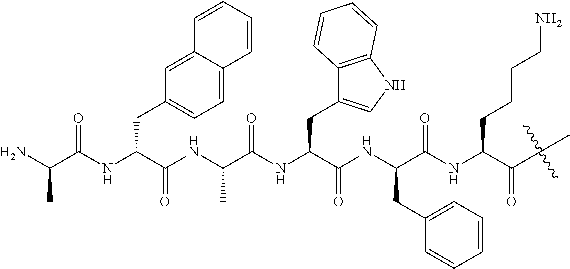 [SMILES: *C(=O)[C@H](CCCCN)NC(=O)[C@@H](Cc1ccccc1)NC(=O)[C@H](Cc1c[nH]c2ccccc12)NC(=O)[C@H](C)NC(=O)[C@@H](Cc1ccc2ccccc2c1)NC(=O)[C@@H](C)N] |
| Relamorelin | 4-[[(2S)-2-[[(2R)-2-[[(2R)-3-(1- Benzothiophen-3-yl)-2- (piperidine-3- carbonylamino)propanoyl]amino]- 3-(1H-indol-3- yl)propanoyl]amino]-3- phenylpropanoyl]amino]piperidine- 4-carboxamide | 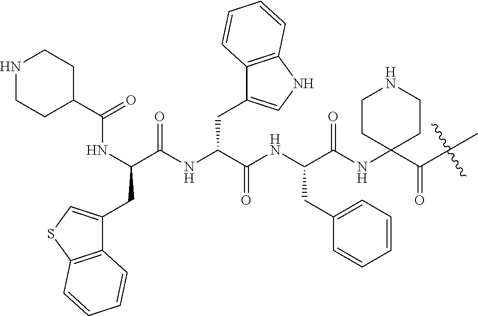 [SMILES: *C(=O)C1(NC(=O)[C@H](Cc2ccccc2)NC(=O)[C@@H](Cc2c[nH]c3ccccc23)NC(=O)[C@@H](Cc2csc3ccccc23)NC(=O)C2CCNCC2)CCNCC1] |
| Tabimorelin | N-[(2E)-5-amino-5-methyl- hexenoyl]-N-methyl-3-(2- naphthyl)alanyl-N,Nα-dimethyl- D-phenylalaninamide | 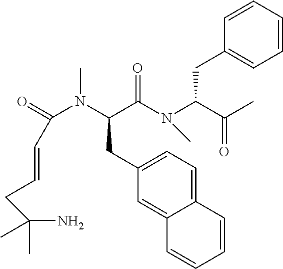 [SMILES: CC(=O)[C@@H](Cc1ccccc1)N(C)C(=O)[C@@H](Cc1ccc2ccccc2c1)N(C)C(=O)/C=C/CC(C)(C)N] |
| SM-130,686 | (+)-(3S)-3-(2-chlorophenyl)-1-[2- (diethylamino)ethyl]-3-hydroxo- 2-oxo-4- (trifluoromethyl)indoline-6- carboxamide | 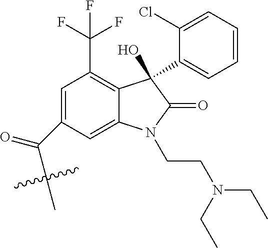 [SMILES: *C(=O)c1cc2c(c(C(F)(F)F)c1)[C@](O)(c1ccccc1Cl)C(=O)N2CCN(CC)CC] |

In some embodiments, a peptidomimetic macrocycle is obtained in more than one isomer, for example due to the configuration of a double bond within the structure of the crosslinker (E vs Z). Such isomers can or cannot be separable by conventional chromatographic methods. In some embodiments, one isomer has improved biological properties relative to the other isomer. In one embodiment, an E crosslinker olefin isomer of a peptidomimetic macrocycle has better solubility, better target affinity, better in vivo or in vitro efficacy or higher helicity relative to its Z counterpart. In another embodiment, a Z crosslinker olefin isomer of a peptidomimetic macrocycle has better solubility, better target affinity, better in vivo or in vitro efficacy or higher helicity relative to its E counterpart.

In some embodiments, a peptidomimetic macrocycle has the Formula (I):

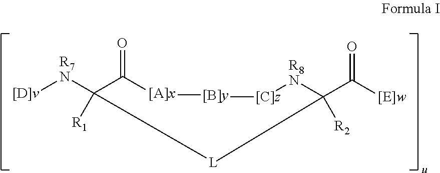

wherein:

each A, C, D, and E is independently a natural or non-natural amino acid;

each B is independently a natural or non-natural amino acid, amino acid analog

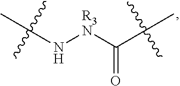

[—NH-L₃-CO—], [—NH-L₃-SO₂—], or [—NH-L₃-];

each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-;

each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;

each L is independently a macrocycle-forming linker;

each L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;

each R₄ is alkylene, alkenylene, alkynylene, heteroalkylene, cycloalkylene, heterocycloalkylene, arylene, or heteroarylene;

each K is independently O, S, SO, SO₂, CO, CO₂, or CONR₃;

n is an integer from 1-5;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;

each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;

each v and w is independently an integer from 1-1000, for example 1-500, 1-200, 1-100, 1-50, 1-40, 1-25, 1-20, 1-15, or 1-10;

u is an integer from 1-10; and

each x, y and z is independently an integer from 0-10.

In some embodiments, L is a macrocycle-forming linker of the formula -L₁-L₂-. In some embodiments, each L₁ and L₂ is independently alkylene, alkenylene, alkynylene, heteroalkylene, cycloalkylene, heterocycloalkylene, arylene, heteroarylene, or [—R₄—K—R₄-]ₙ, each being optionally substituted with R₅; each R₄ is independently alkylene, alkenylene, alkynylene, heteroalkylene, cycloalkylene, heterocycloalkylene, arylene, or heteroarylene; each K is O, S, SO, SO₂, CO, CO₂, or CONR₃; and n is an integer from 1-5.

In one example, at least one of R₁ and R₂ is alkyl, unsubstituted or substituted with halo-. In another example, each R₁ and R₂ is independently an alkyl group, unsubstituted or substituted with halo-. In some embodiments, at least one of R₁ and R₂ is methyl. In other embodiments, R₁ and R₂ are methyl.

In some embodiments, w is an integer from 3-10, for example 3-6, 3-8, 6-8, or 6-10. In some embodiments, w is 3. In other embodiments, w is 6. In some embodiments, v is an integer from 1-1000, for example 1-500, 1-200, 1-100, 1-50, 1-30, 1-20, or 1-10. In some embodiments, v is 2.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments, x+y+z is at least 2. In other embodiments, x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g., Gln-Asp-Ala, as well as embodiments where the amino acids are identical, e.g., Gln-Gln-Gln. This applies for any value of x, y, or z in the indicated ranges. Similarly, when u is greater than 1, each compound may encompass peptidomimetic macrocycles which are the same or different. For example, a compound may comprise peptidomimetic macrocycles comprising different linker lengths or chemical compositions.

In some embodiments, the peptidomimetic macrocycle comprises a secondary structure which is a helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

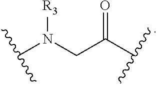

In other embodiments, the length of the macrocycle-forming linker L as measured from a first Cα to a second Cα is selected to stabilize a desired secondary peptide structure, such as a helix formed by residues of the peptidomimetic macrocycle including, but not necessarily limited to, those between the first Cα to a second Ca.

In some embodiments, a peptidomimetic macrocycle of Formula (I) has Formula (Ic):

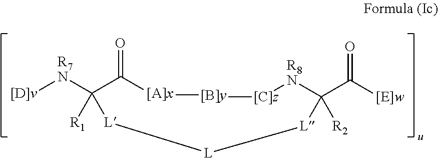

wherein:

each A, C, D, and E is independently a natural or non-natural amino acid;

each B is independently a natural or non-natural amino acid, amino acid analog,

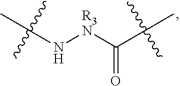

[—NH-L₃-CO—], [—NH-L₃-SO₂—], or [—NH-L₃-];

each L is independently a macrocycle-forming linker;

each L′ is independently alkylene, alkenylene, alkynylene, heteroalkylene, cycloalkylene, heterocycloalkylene, arylene, or heteroarylene, each being optionally substituted with R₅, or a bond, or together with R₁ and the atom to which both R₁ and L′ are bound forms a ring;

each L″ is independently alkylene, alkenylene, alkynylene, heteroalkylene, cycloalkylene, heterocycloalkylene, arylene, or heteroarylene, each being optionally substituted with R₅, or a bond, or together with R₂ and the atom to which both R₂ and L″ are bound forms a ring;

each R₁ is independently —H, alkyl, alkenyl, alkynyl, arylalkyl, cycloalkyl, cycloalkylalkyl, heteroalkyl, or heterocycloalkyl, unsubstituted or substituted with halo-, or together with L′ and the atom to which both R₁ and L′ are bound forms a ring;

each R₂ is independently —H, alkyl, alkenyl, alkynyl, arylalkyl, cycloalkyl, cycloalkylalkyl, heteroalkyl, or heterocycloalkyl, unsubstituted or substituted with halo-, or together with L″ and the atom to which both R₂ and L″ are bound forms a ring;

each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;

each L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;

each R₄ is alkylene, alkenylene, alkynylene, heteroalkylene, cycloalkylene, heterocycloalkylene, arylene, or heteroarylene;

each K is O, S, SO, SO₂, CO, CO₂, or CONR₃;

n is an integer from 1-5;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;

each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;

each v and w is independently an integer from 1-1000, for example 1-500, 1-200, 1-100, 1-50, 1-40, 1-25, 1-20, 1-15, or 1-10;

u is an integer from 1-10; and

each x, y and z is independently an integer from 0-10.

In some embodiments, L is a macrocycle-forming linker of the formula -L₁-L₂-. In some embodiments, each L₁ and L₂ is independently alkylene, alkenylene, alkynylene, heteroalkylene, cycloalkylene, heterocycloalkylene, arylene, heteroarylene, or [—R₄—K—R₄-]ₙ, each being optionally substituted with R₅; each R₄ is independently alkylene, alkenylene, alkynylene, heteroalkylene, cycloalkylene, heterocycloalkylene, arylene, or heteroarylene; each K is O, S, SO, SO₂, CO, CO₂, or CONR₃; and n is an integer from 1-5.

In one example, at least one of R₁ and R₂ is alkyl, unsubstituted or substituted with halo-. In another example, each R₁ and R₂ is independently an alkyl group that is unsubstituted or substituted with halo-. In some embodiments, at least one of R₁ and R₂ is methyl. In other embodiments, R₁ and R₂ are methyl.

In some embodiments, w is an integer from 3-10, for example 3-6, 3-8, 6-8, or 6-10. In some embodiments, w is 3. In other embodiments, w is 6. In some embodiments, v is an integer from 1-1000, for example 1-500, 1-200, 1-100, 1-50, 1-30, 1-20, or 1-10. In some embodiments, v is 2.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments, x+y+z is at least 2. In other embodiments, x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g., Gln-Asp-Ala, as well as embodiments where the amino acids are identical, e.g., Gln-Gln-Gln. This applies for any value of x, y, or z in the indicated ranges. Similarly, when u is greater than 1, each compound may encompass peptidomimetic macrocycles which are the same or different. For example, a compound may comprise peptidomimetic macrocycles comprising different linker lengths or chemical compositions.

In some embodiments, the peptidomimetic macrocycle comprises a secondary structure which is a helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

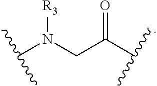

In other embodiments, the length of the macrocycle-forming linker L as measured from a first Cα to a second Cα is selected to stabilize a desired secondary peptide structure, such as a helix formed by residues of the peptidomimetic macrocycle including, but not necessarily limited to, those between the first Cα to a second Ca.

For example, u is 1. For example, u is 2.

In some embodiments, the sum of x+y+z is 2, 3 or 6, for example 3 or 6.

In some embodiments, the peptidomimetic macrocycle of Formula (I) has the Formula:

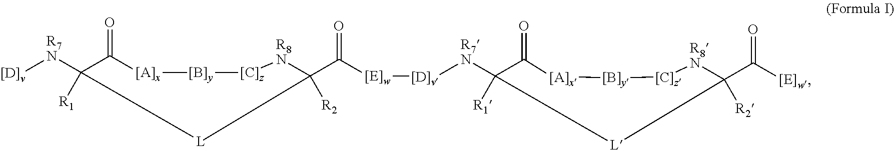

wherein:

each A, C, D, and E is independently an amino acid;

each B is independently an amino acid,

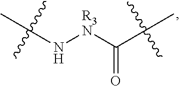

[—NH-L₃-CO—], [—NH-L₃-SO₂—], or [—NH-L₃-];

L is a macrocycle-forming linker of the formula -L₁-L₂-;

L′ is a macrocycle-forming linker of the formula -L₁′-L₂′-;

and wherein A, B, C, D, and E, taken together with the crosslinked amino acids connected by the macrocycle-forming linkers L and L′, form the amino acid sequence of the peptidomimetic macrocycle;

each R₁, R₁′, R₂, and R₂′ is independently —H, alkyl, alkenyl, alkynyl, arylalkyl, cycloalkyl, cycloalkylalkyl, heteroalkyl, or heterocycloalkyl, unsubstituted or substituted with halo-; each L₁, L₁′, L₂, L₂′, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;

each K is independently O, S, SO, SO₂, CO, CO₂, or CONR₃;

each R₇ and R₇′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;

each R₈′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;

each v, v′, w, and w′ is independently an integer from 1-1000, for example 1-500, 1-200, 1-100, 1-50, 1-40, 1-25, 1-20, 1 to 15, or 1 to 10;

each x, x′, y, y′, z, and z′ is independently an integer from 0-10; and

n is an integer from 1-5. In some embodiments, the sum of x′+y′+z′ is 2, 3 or 6, for example 3 or 6.

In some embodiments of any of the peptidomimetic macrocycles described herein, each K is O, S, SO, SO₂, CO, or CO₂.

In one example, at least one of R₁ and R₂ is alkyl, unsubstituted or substituted with halo-. In another example, each R₁ and R₂ is independently an alkyl group that is unsubstituted or substituted with halo-. In some embodiments, at least one of R₁ and R₂ is methyl. In other embodiments, R₁ and R₂ are methyl.

In some embodiments, each w and w′ is independently an integer from 3-10, for example 3-6, 3-8, 6-8, or 6-10. In some embodiments, each w and w′ is independently 3. In other embodiments, each w and w′ is independently 6. In some embodiments, each v and v′ is independently an integer from 1-1000, for example 1-500, 1-200, 1-100, 1-50, 1-30, 1-20, or 1-10. In some embodiments, each v and v′ is independently 2.

In some embodiments, each w and w′ is independently between 1 and 1000. For example, the first amino acid represented by E comprises a small hydrophobic side chain. In some embodiments, each w and w′ is independently between 2 and 1000. For example, the second amino acid represented by E comprises a small hydrophobic side chain. In some embodiments, each w and w′ is independently between 3 and 1000. For example, the third amino acid represented by E comprises a small hydrophobic side chain. For example, the third amino acid represented by E comprises a small hydrophobic side chain. In some embodiments, each w and w′ is independently between 4 and 1000. In some embodiments, w is between 5 and 1000. In some embodiments, each w and w′ is independently between 6 and 1000. In some embodiments, each w and w′ is independently between 7 and 1000. In some embodiments, each w and w′ is independently between 8 and 1000.

In some embodiments of the invention, the sum of x+y+z is at least 3, and/or the sum of x′+y′+z′ is at least 3. In other embodiments of the invention, the sum of x+y+z is 1, 2, 3, 4, 5, 6, 7, 8, 9 or 10 (for example 2, 3 or 6) and/or the sum of x′+y′+z′ is 1, 2, 3, 4, 5, 6, 7, 8, 9 or 10 (for example 2, 3 or 6).

Each occurrence of A, B, C, D or E in a macrocycle or macrocycle precursor is independently selected. For example, a sequence represented by the formula [A]ₓ, when x is 3, encompasses embodiments where the amino acids are not identical, e.g., Gln-Asp-Ala as well as embodiments where the amino acids are identical, e.g., Gln-Gln-Gln. This applies for any value of x, y, or z in the indicated ranges. Similarly, when u is greater than 1, each compound may encompass peptidomimetic macrocycles which are the same or different. For example, a compound may comprise peptidomimetic macrocycles comprising different linker lengths or chemical compositions.

In some embodiments, the peptidomimetic macrocycle comprises a helical secondary structure and R₈ is —H, allowing for intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

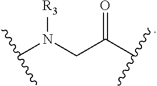

In other embodiments, the length of the macrocycle-forming linker L as measured from a first Cα to a second Cα is selected to stabilize a desired secondary peptide structure, such as an α-helix formed by residues of the peptidomimetic macrocycle including, but not limited to, those between the first Cα to a second Ca.

In one embodiment, the peptidomimetic macrocycle of Formula (I) is

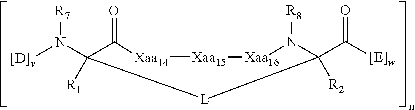

or a pharmaceutically-acceptable salt thereof wherein:
each of Xaa₁₄, Xaa₁₅, and Xaa₁₆ is independently an amino acid, wherein at least one, two, or each of Xaa₁₄, Xaa₁₅, and Xaa₁₆ are the same amino acid as the amino acid at the corresponding position of the sequence Xaa₁₃-Leu₁₄-Ala/Gly/Abu₁₅-Gln/Ala/Glu/Nle/Ser₁₆-Xaa₁₇, where each of Xaa₁₃ and Xaa₁₇ is independently an amino acid.

In one embodiment, the peptidomimetic macrocycle of Formula (I) is

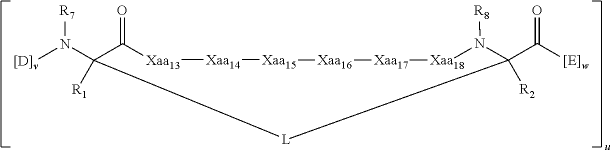

or a pharmaceutically-acceptable salt thereof wherein:
each of Xaa₁₃, Xaa₁₄, Xaa₁₅, Xaa₁₆, Xaa₁₇, and Xaa₁₈ is independently an amino acid, wherein at least one, two, three, four, five, or each of Xaa₁₃, Xaa₁₄, Xaa₁₅, Xaa₁₆, Xaa₁₇, and Xaa₁₈, are the same amino acid as the amino acid at the corresponding position of the sequence Xaa₁₂-Val₁₃-Leu₁₄-Ala/Gly₁₅-Gln/Ala₁₆-Leu₁₇-Ser₁₈-Xaa₁₉, where each of Xaa₁₂ and Xaa₁₉ is independently an amino acid;
each D and E is independently an amino acid.

In one embodiment, the peptidomimetic macrocycle of Formula (I) is:

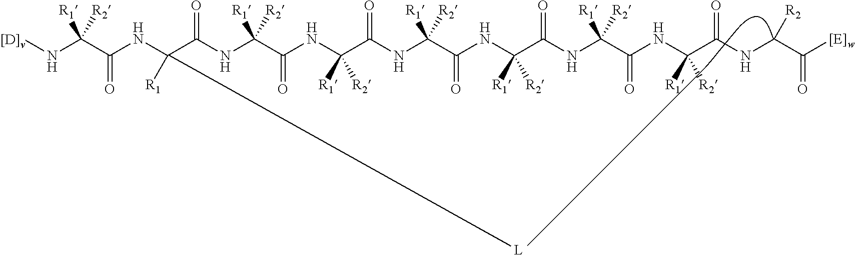

wherein each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle of Formula (I) is:

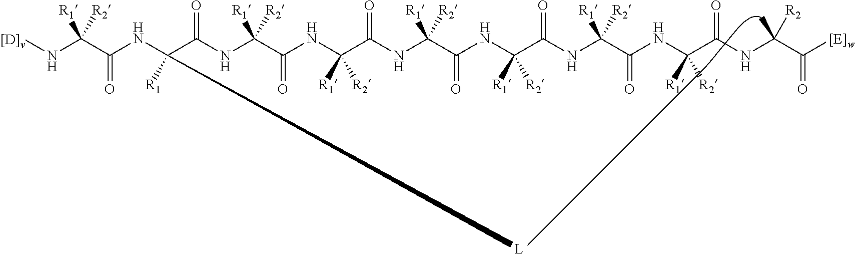

wherein each R₁′ and R₂′ is independently an amino acid.

In one embodiment, the peptidomimetic macrocycle of Formula (I) is:

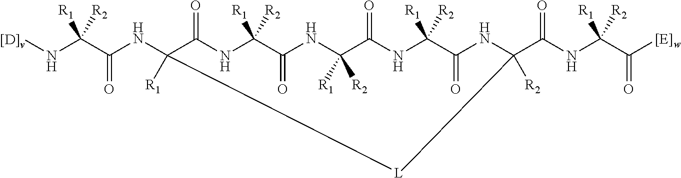

wherein each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-.

In related embodiments, the peptidomimetic macrocycle comprises a structure of Formula (I) which is:

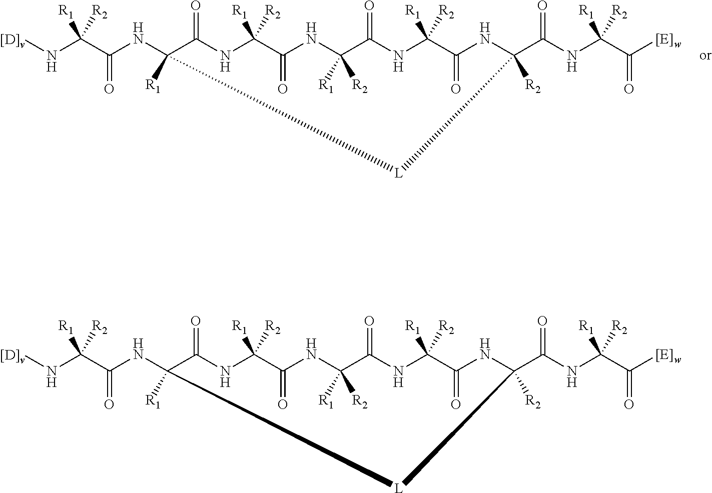

In other embodiments, the peptidomimetic macrocycle of Formula (I) is a compound of any of the formulas shown below:

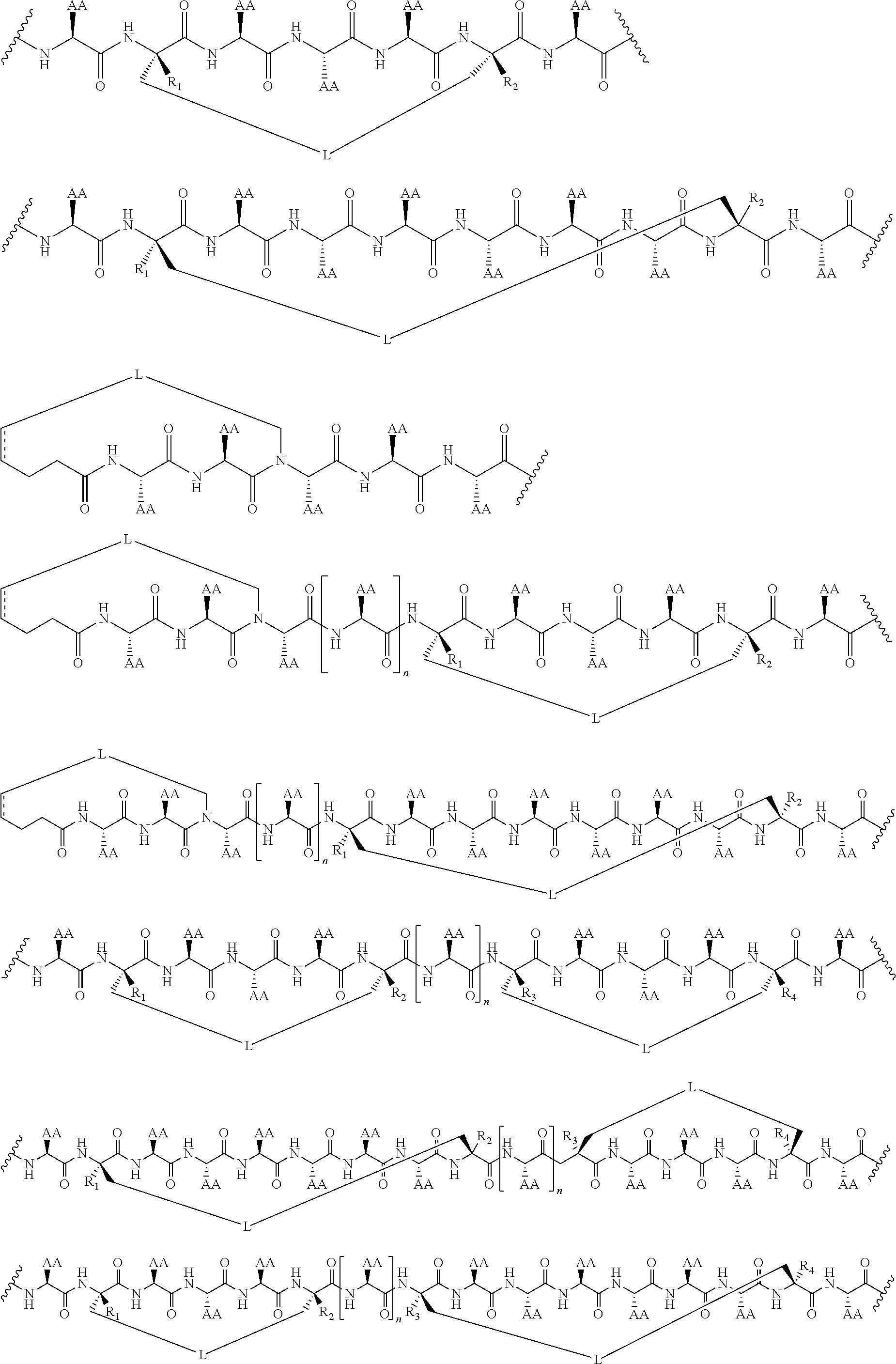

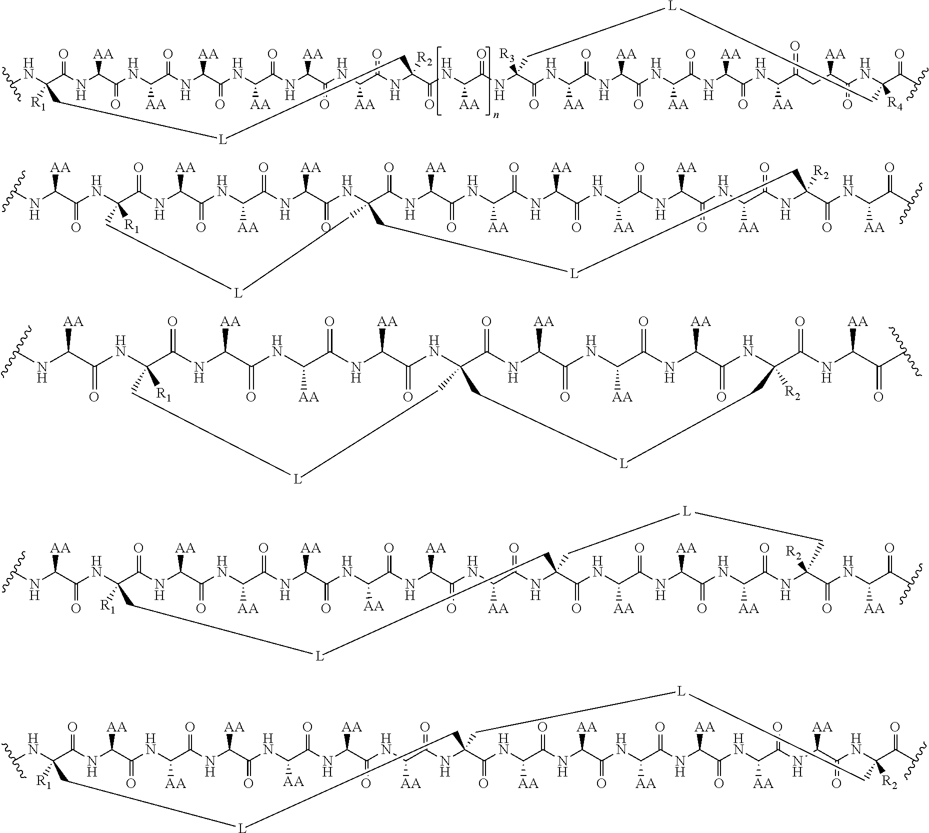

wherein “AA” represents any natural or non-natural amino acid side chain and “ [symbol] ” is [D]ᵥ, [E]_{w} as defined above, and n is an integer between 0 and 20, 50, 100, 200, 300, 400 or 500. In some embodiments, the substituent “n” shown in the preceding paragraph is 0. In other embodiments, the substituent “n” shown in the preceding paragraph is less than 50, 40, 30, 20, 10, or 5.

Exemplary embodiments of the macrocycle-forming linker L are shown below.

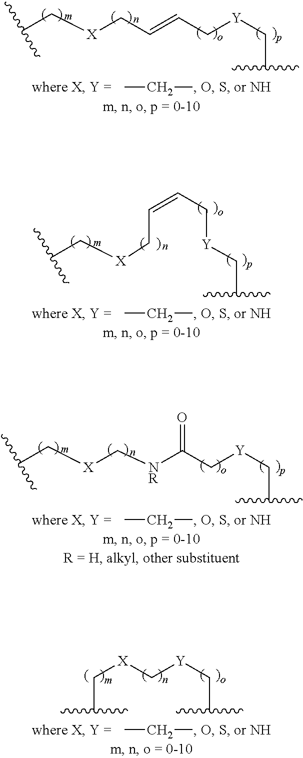

In some embodiments, the peptidomimetic macrocycles have the Formula (I):

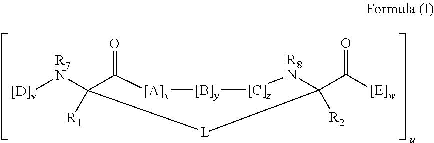

wherein:

each A, C, D, and E is independently a natural or non-natural amino acid;

each B is independently a natural or non-natural amino acid, amino acid analog,

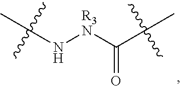

[—NH-L₃-CO—], [—NH-L₃-SO₂—], or [—NH-L₃-];

each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-;

each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;

each L is independently a macrocycle-forming linker of the formula

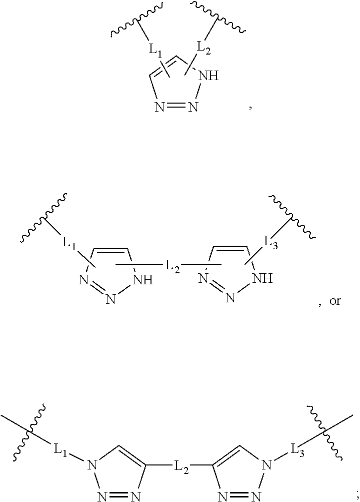

each L₁, L₂ and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;

each R₄ is independently alkylene, alkenylene, alkynylene, heteroalkylene, cycloalkylene, heterocycloalkylene, arylene, or heteroarylene;

each K is independently O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;

each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;

each v and w is independently an integer from 1-1000;

u is an integer from 1-10;

each x, y and z is independently an integer from 0-10; and

n is an integer from 1-5.

In one example, at least one of R₁ and R₂ is alkyl, unsubstituted or substituted with halo-. In another example, each R₁ and R₂ is independently an alkyl group that is unsubstituted or substituted with halo-. In some embodiments, at least one of R₁ and R₂ is methyl. In other embodiments, R₁ and R₂ are methyl.

In some embodiments, w is an integer from 3-10, for example 3-6, 3-8, 6-8, or 6-10. In some embodiments, w is 3. In other embodiments, w is 6. In some embodiments, v is an integer from 1-1000, for example 1-500, 1-200, 1-100, 1-50, 1-30, 1-20, or 1-10. In some embodiments, v is 2.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments, x+y+z is at least 2. In other embodiments, x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g., Gln-Asp-Ala, as well as embodiments where the amino acids are identical, e.g., Gln-Gln-Gln. This applies for any value of x, y, or z in the indicated ranges.

In some embodiments, lipidating or PEGylating a peptidomimetic macrocycle increases bioavailability, increases blood circulation, alters pharmacokinetics, decreases immunogenicity and/or decreases the needed frequency of administration.

In other embodiments, at least one of [D] and [E] in the compound of Formula (I) represents a moiety comprising an additional macrocycle-forming linker such that the peptidomimetic macrocycle comprises at least two macrocycle-forming linkers. In a specific embodiment, a peptidomimetic macrocycle comprises two macrocycle-forming linkers.

In the peptidomimetic macrocycles of the invention, any of the macrocycle-forming linkers described herein may be used in any combination with any of the sequences shown in Table 1a, 1b, 2a, 2b, or 2c, and also with any of the R— substituents indicated herein.

In some embodiments, the peptidomimetic macrocycle comprises at least one α-helix motif. For example, A, B and/or C in the compound of Formula (I) include one or more α-helices. As a general matter, α-helices include between 3 and 4 amino acid residues per turn. In some embodiments, the α-helix of the peptidomimetic macrocycle includes 1 to 5 turns and, therefore, 3 to 20 amino acid residues. In specific embodiments, the α-helix includes 1 turn, 2 turns, 3 turns, 4 turns, or 5 turns. In some embodiments, the macrocycle-forming linker stabilizes an α-helix motif included within the peptidomimetic macrocycle. Thus, in some embodiments, the length of the macrocycle-forming linker L from a first Cα to a second Cα is selected to increase the stability of an α-helix. In some embodiments, the macrocycle-forming linker spans from 1 turn to 5 turns of the α-helix. In some embodiments, the macrocycle-forming linker spans approximately 1 turn, 2 turns, 3 turns, 4 turns, or 5 turns of the α-helix. In some embodiments, the length of the macrocycle-forming linker is approximately 5 Å to 9 Å per turn of the α-helix, or approximately 6 Å to 8 Å per turn of the α-helix. Where the macrocycle-forming linker spans approximately 1 turn of an α-helix, the length is equal to approximately 5 carbon-carbon bonds to 13 carbon-carbon bonds, approximately 7 carbon-carbon bonds to 11 carbon-carbon bonds, or approximately 9 carbon-carbon bonds. Where the macrocycle-forming linker spans approximately 2 turns of an α-helix, the length is equal to approximately 8 carbon-carbon bonds to 16 carbon-carbon bonds, approximately 10 carbon-carbon bonds to 14 carbon-carbon bonds, or approximately 12 carbon-carbon bonds. Where the macrocycle-forming linker spans approximately 3 turns of an α-helix, the length is equal to approximately 14 carbon-carbon bonds to 22 carbon-carbon bonds, approximately 16 carbon-carbon bonds to 20 carbon-carbon bonds, or approximately 18 carbon-carbon bonds.

Where the macrocycle-forming linker spans approximately 4 turns of an α-helix, the length is equal to approximately 20 carbon-carbon bonds to 28 carbon-carbon bonds, approximately 22 carbon-carbon bonds to 26 carbon-carbon bonds, or approximately 24 carbon-carbon bonds. Where the macrocycle-forming linker spans approximately 5 turns of an α-helix, the length is equal to approximately 26 carbon-carbon bonds to 34 carbon-carbon bonds, approximately 28 carbon-carbon bonds to 32 carbon-carbon bonds, or approximately 30 carbon-carbon bonds. Where the macrocycle-forming linker spans approximately 1 turn of an α-helix, the linkage contains approximately 4 atoms to 12 atoms, approximately 6 atoms to 10 atoms, or approximately 8 atoms. Where the macrocycle-forming linker spans approximately 2 turns of the α-helix, the linkage contains approximately 7 atoms to 15 atoms, approximately 9 atoms to 13 atoms, or approximately 11 atoms. Where the macrocycle-forming linker spans approximately 3 turns of the α-helix, the linkage contains approximately 13 atoms to 21 atoms, approximately 15 atoms to 19 atoms, or approximately 17 atoms. Where the macrocycle-forming linker spans approximately 4 turns of the α-helix, the linkage contains approximately 19 atoms to 27 atoms, approximately 21 atoms to 25 atoms, or approximately 23 atoms. Where the macrocycle-forming linker spans approximately 5 turns of the α-helix, the linkage contains approximately 25 atoms to 33 atoms, approximately 27 atoms to 31 atoms, or approximately 29 atoms. Where the macrocycle-forming linker spans approximately 1 turn of the α-helix, the resulting macrocycle forms a ring containing approximately 17 members to 25 members, approximately 19 members to 23 members, or approximately 21 members. Where the macrocycle-forming linker spans approximately 2 turns of the α-helix, the resulting macrocycle forms a ring containing approximately 29 members to 37 members, approximately 31 members to 35 members, or approximately 33 members. Where the macrocycle-forming linker spans approximately 3 turns of the α-helix, the resulting macrocycle forms a ring containing approximately 44 members to 52 members, approximately 46 members to 50 members, or approximately 48 members. Where the macrocycle-forming linker spans approximately 4 turns of the α-helix, the resulting macrocycle forms a ring containing approximately 59 members to 67 members, approximately 61 members to 65 members, or approximately 63 members. Where the macrocycle-forming linker spans approximately 5 turns of the α-helix, the resulting macrocycle forms a ring containing approximately 74 members to 82 members, approximately 76 members to 80 members, or approximately 78 members.

In some embodiments, L is a macrocycle-forming linker of the formula

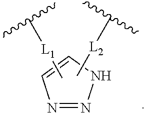

In some embodiments, L is a macrocycle-forming linker of the formula

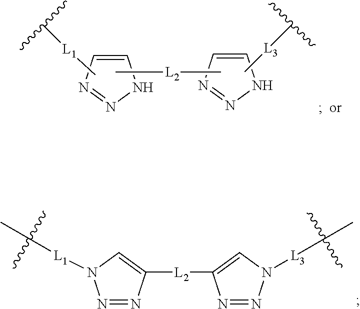

or a tautomer thereof.

Exemplary embodiments of such macrocycle-forming linkers L are shown below.

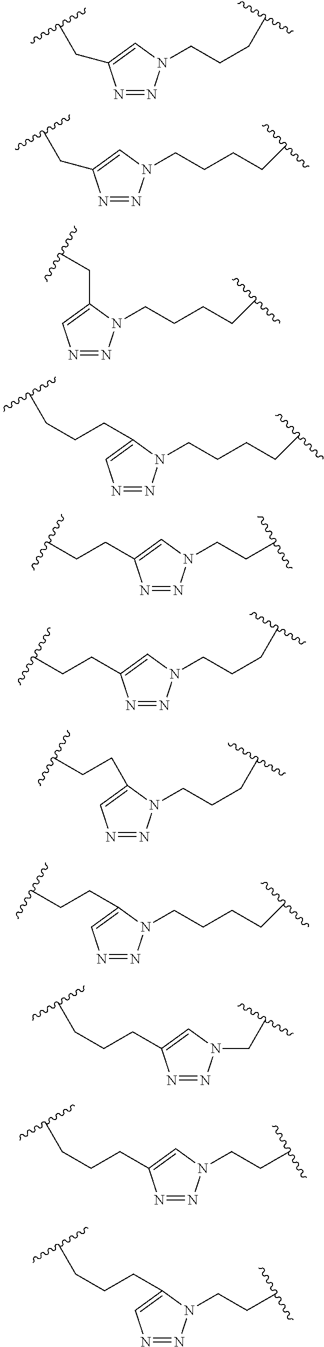

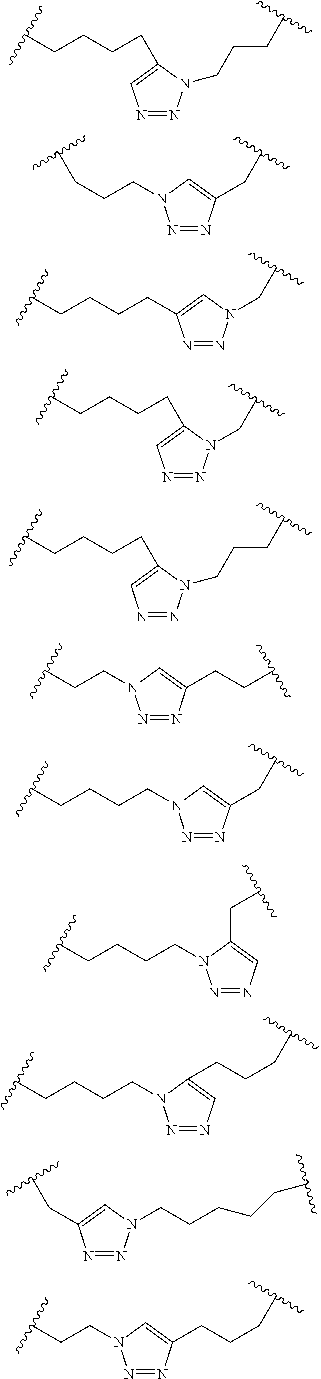

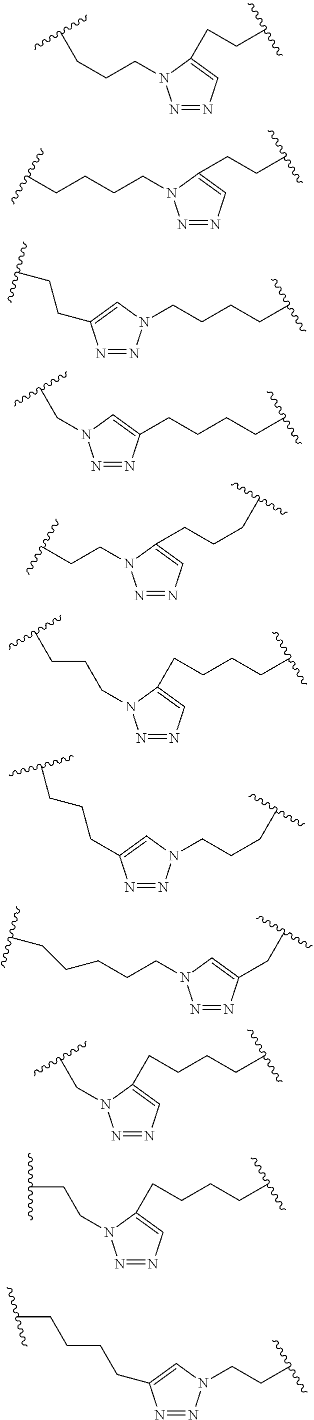

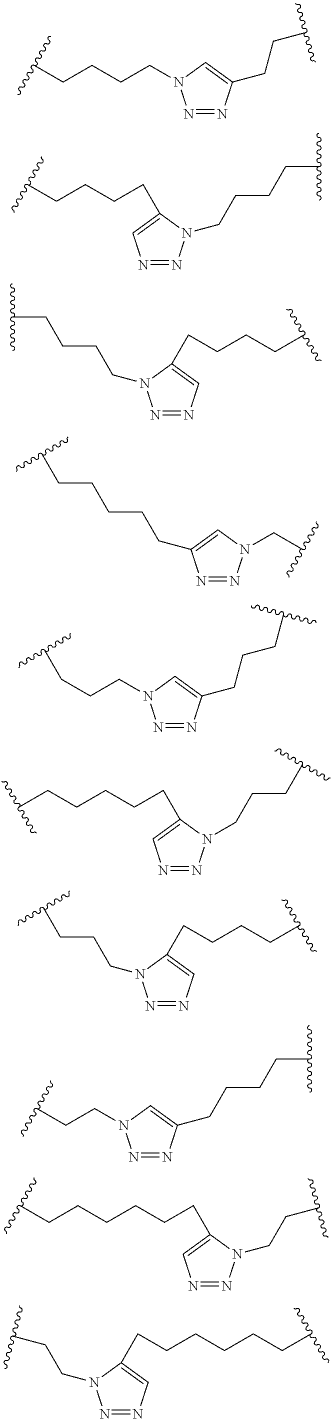

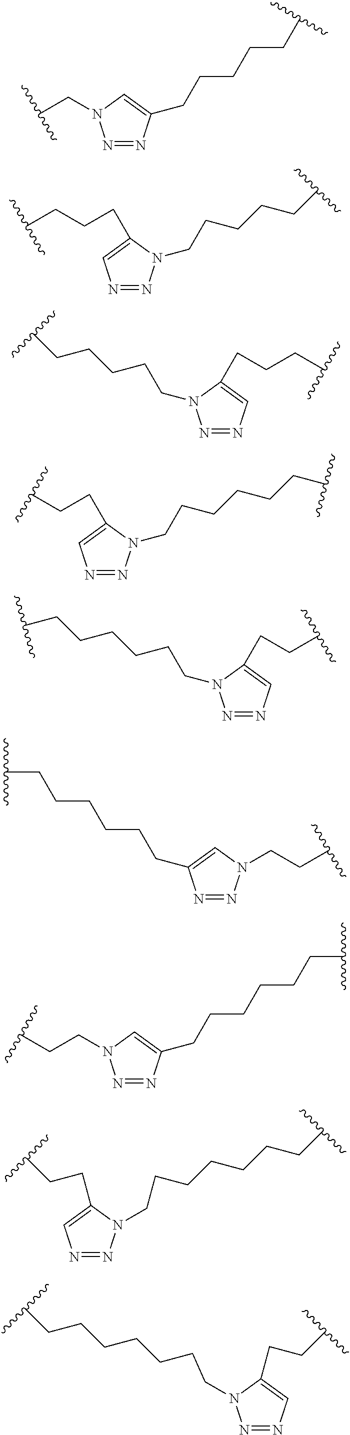

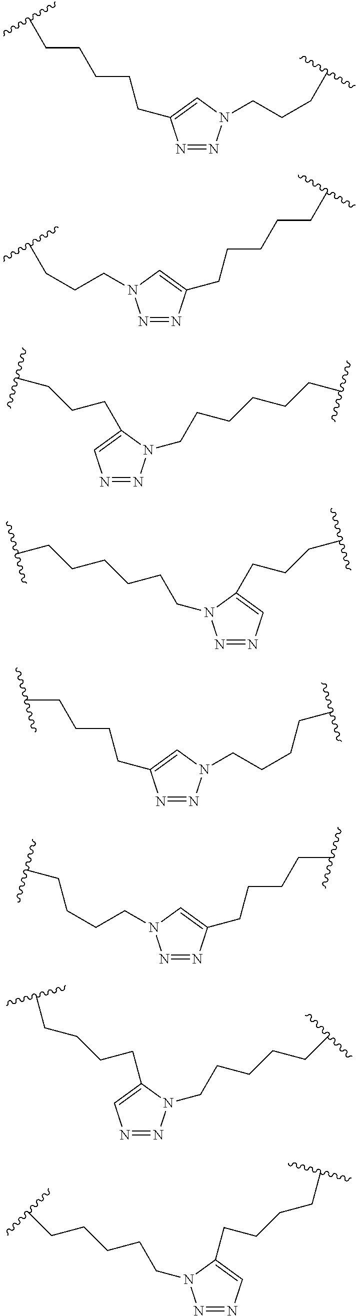

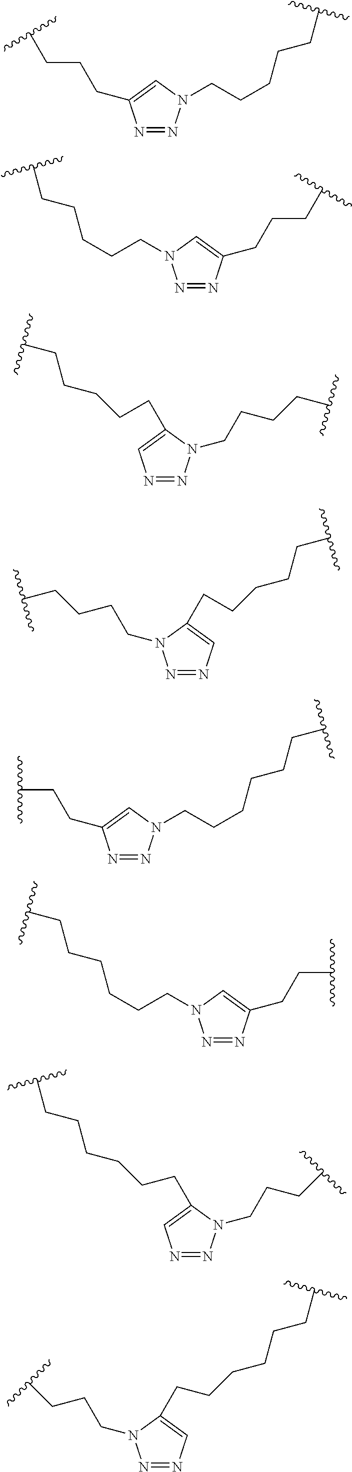

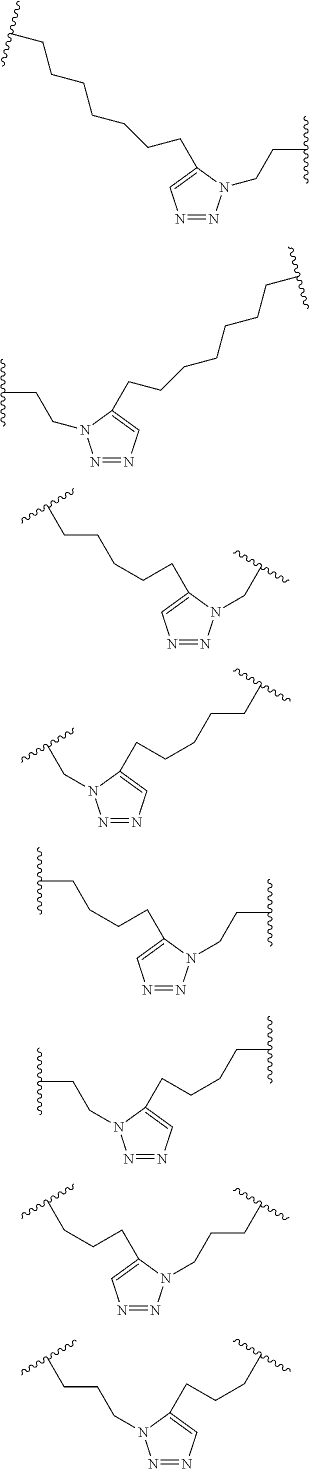

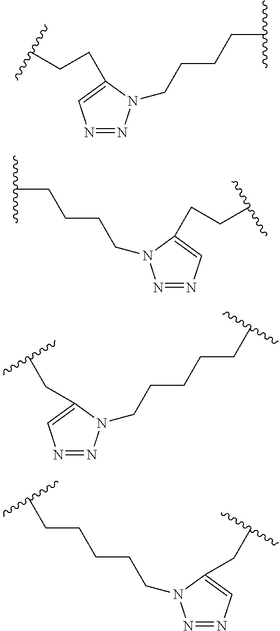 [SMILES: *CCCCn1nncc1CC* ; *CCCCn1nncc1CC* ; *CCCCCn1nncc1C* ; *CCCCCn1nncc1C*]

In other embodiments, the invention provides peptidomimetic macrocycles of Formula (III):

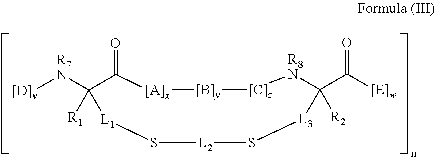

wherein:

each A, C, D, and E is independently a natural or non-natural amino acid;

each B is independently a natural or non-natural amino acid, amino acid analog,

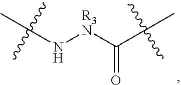

[—NH-L₄-CO—], [—NH-L₄-SO₂—], or [—NH-L₄-];

each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-;

each R₃ is independently hydrogen, alkyl, alkenyl, alkynyl, arylalkyl, heteroalkyl, cycloalkyl, heterocycloalkyl, cycloalkylalkyl, aryl, or heteroaryl, unsubstituted or substituted with R₅;

each L₁, L₂, L₃ and L₄ is independently alkylene, alkenylene, alkynylene, heteroalkylene, cycloalkylene, heterocycloalkylene, arylene, heteroarylene or [—R₄—K—R₄-]ₙ, each being unsubstituted or substituted with R₅;

each K is independently O, S, SO, SO₂, CO, CO₂, or CONR₃;

each R₄ is independently alkylene, alkenylene, alkynylene, heteroalkylene, cycloalkylene, heterocycloalkylene, arylene, or heteroarylene;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

each R₇ is independently —H, alkyl, alkenyl, alkynyl, arylalkyl, cycloalkyl, heteroalkyl, cycloalkylalkyl, heterocycloalkyl, aryl, or heteroaryl, unsubstituted or substituted with R₅, or part of a cyclic structure with a D residue;

each R₈ is —H, alkyl, alkenyl, alkynyl, arylalkyl, cycloalkyl, heteroalkyl, cycloalkylalkyl, heterocycloalkyl, aryl, or heteroaryl, unsubstituted or substituted with R₅, or part of a cyclic structure with an E residue;

each v and w is independently an integer from 1-1000;

u is an integer from 1-10;

each x, y and z is independently an integer from 0-10; and

n is an integer from 1-5.

In one example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl.

In some embodiments, w is an integer from 3-10, for example 3-6, 3-8, 6-8, or 6-10. In some embodiments, w is 3. In other embodiments, w is 6. In some embodiments, v is an integer from 1-1000, for example 1-500, 1-200, 1-100, 1-50, 1-30, 1-20, or 1-10. In some embodiments, v is 2.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments, x+y+z is at least 2. In other embodiments, x+y+z is 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g., Gln-Asp-Ala, as well as embodiments where the amino acids are identical, e.g., Gln-Gln-Gln. This applies for any value of x, y, or z in the indicated ranges.

In some embodiments, each of the first two amino acid represented by E comprises an uncharged side chain or a negatively charged side chain. In some embodiments, each of the first three amino acid represented by E comprises an uncharged side chain or a negatively charged side chain. In some embodiments, each of the first four amino acid represented by E comprises an uncharged side chain or a negatively charged side chain.

In some embodiments, the first C-terminal amino acid and/or the second C-terminal amino acid represented by E comprise a hydrophobic side chain. For example, the first C-terminal amino acid and/or the second C-terminal amino acid represented by E comprises a hydrophobic side chain, for example a small hydrophobic side chain. In some embodiments, the first C-terminal amino acid, the second C-terminal amino acid, and/or the third C-terminal amino acid represented by E comprise a hydrophobic side chain. For example, the first C-terminal amino acid, the second C-terminal amino acid, and/or the third C-terminal amino acid represented by E comprises a hydrophobic side chain, for example a small hydrophobic side chain.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments, the peptidomimetic macrocycle comprises a secondary structure which is a helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

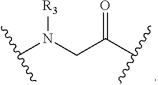

In other embodiments, the length of the macrocycle-forming linker [-L₁-S-L₂-S-L₃-] as measured from a first Cα to a second Cα is selected to stabilize a desired secondary peptide structure, such as a helix (including, but not limited to a 3₁₀ helix or an α-helix) formed by residues of the peptidomimetic macrocycle including, but not necessarily limited to, those between the first Cα to a second Cα.

Macrocycles or macrocycle precursors are synthesized, for example, by solution phase or solid-phase methods, and can contain both naturally-occurring and non-naturally-occurring amino acids. See, for example, Hunt, “The Non-Protein Amino Acids” in Chemistry and Biochemistry of the Amino Acids, edited by G. C. Barrett, Chapman and Hall, 1985. In some embodiments, the thiol moieties are the side chains of the amino acid residues L-cysteine, D-cysteine, α-methyl-L cysteine, α-methyl-D-cysteine, L-homocysteine, D-homocysteine, α-methyl-L-homocysteine or α-methyl-D-homocysteine. A bis-alkylating reagent is of the general formula X-L₂-Y wherein L₂ is a linker moiety and X and Y are leaving groups that are displaced by —SH moieties to form bonds with L₂. In some embodiments, X and Y are halogens such as I, Br, or Cl.

In some embodiments, lipidating or PEGylating a peptidomimetic macrocycle increases bioavailability, increases blood circulation, alters pharmacokinetics, decreases immunogenicity and/or decreases the needed frequency of administration.

In other embodiments, at least one of [D] and [E] in the compound of Formula (I), (II), or (III) represents a moiety comprising an additional macrocycle-forming linker such that the peptidomimetic macrocycle comprises at least two macrocycle-forming linkers. In a specific embodiment, a peptidomimetic macrocycle comprises two macrocycle-forming linkers.

In the peptidomimetic macrocycles, any of the macrocycle-forming linkers described herein may be used in any combination with any of the sequences shown in Tables 1a, 1b, 2a, 2b, or 2c, and also with any of the R— substituents indicated herein.

In other embodiments, the invention provides peptidomimetic macrocycles of Formula (II) or (IIa):

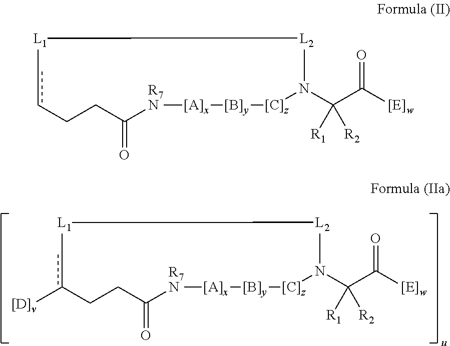

wherein:

each A, C, D, and E is independently an amino acid;

each B is independently an amino acid,

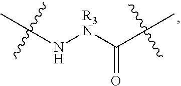

[—NH-L₃-CO—], [—NH-L₃-SO₂—], or [—NH-L₃-];

each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-, or part of a cyclic structure with an E residue;

each R₃ is independently hydrogen, alkyl, alkenyl, alkynyl, arylalkyl, heteroalkyl, cycloalkyl, heterocycloalkyl, cycloalkylalkyl, aryl, or heteroaryl, optionally substituted with R₅;

each L₁, L₂, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;

wherein A, B, C, D, and E, taken together with the crosslinked amino acids connected by the macrocycle-forming linker -L₁-L₂-, form the amino acid sequence of the peptidomimetic macrocycle which is at least about 60%, 65%, 70%, 75%, 80%, 85%, 90%, 95%, 96%, 97%, 98%, or 99% identical to an amino acid sequence chosen from the group consisting of the amino acid sequences in Table 1a, 1b, 2a, 2b, or 2c;

each R₄ is independently alkylene, alkenylene, alkynylene, heteroalkylene, cycloalkylene, heterocycloalkylene, arylene, or heteroarylene;

each K is independently O, S, SO, SO₂, CO, CO₂, or CONR₃;

each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;

each R₆ is independently —H, alkyl, alkenyl, alkynyl, arylalkyl, cycloalkylalkyl, heterocycloalkyl, a fluorescent moiety, a radioisotope or a therapeutic agent;

each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅;

each v and w is independently an integer from 1-1000, for example 1-100;

u is an integer from 1-10, for example u is 1-3;

each x, y and z is independently an integer from 0-10; and each n is independently an integer from 1-5.

In one example, at least one of R₁ and R₂ is alkyl, unsubstituted or substituted with halo-. In another example, each R₁ and R₂ is independently an alkyl group that is unsubstituted or substituted with halo-. In some embodiments, at least one of R₁ and R₂ is methyl. In other embodiments, R₁ and R₂ are methyl.

In some embodiments, w is an integer from 3-10, for example 3-6, 3-8, 6-8, or 6-10. In some embodiments, w is 3. In other embodiments, w is 6. In some embodiments, v is an integer from 1-1000, for example 1-500, 1-200, 1-100, 1-50, 1-30, 1-20, or 1-10. In some embodiments, v is 2.

In some embodiments, w is between 1 and 1000. For example, the first amino acid represented by E comprises a small hydrophobic side chain. In some embodiments, w is between 2 and 1000. For example, the second amino acid represented by E comprises a small hydrophobic side chain. In some embodiments, w is between 3 and 1000. For example, the third amino acid represented by E comprises a small hydrophobic side chain. For example, the third amino acid represented by E comprises a small hydrophobic side chain. In some embodiments, w is between 4 and 1000. In some embodiments, w is between 5 and 1000. In some embodiments, w is between 6 and 1000. In some embodiments, w is between 7 and 1000. In some embodiments, w is between 8 and 1000.

In some embodiments of the invention, the sum of x+y+z is at least 1. In other embodiments of the invention, the sum of x+y+z is at least 2. In other embodiments of the invention, the sum of x+y+z is 1, 2, 3, 4, 5, 6, 7, 8, 9 or 10. Each occurrence of A, B, C, D or E in a macrocycle or macrocycle precursor is independently selected. For example, a sequence represented by the formula [A]ₓ, when x is 3, encompasses embodiments where the amino acids are not identical, e.g., Gln-Asp-Ala as well as embodiments where the amino acids are identical, e.g., Gln-Gln-Gln. This applies for any value of x, y, or z in the indicated ranges.

In some embodiments, the peptidomimetic macrocycle comprises a secondary structure which is an α-helix and R₈ is —H, allowing intrahelical hydrogen bonding. In some embodiments, at least one of A, B, C, D or E is an α,α-disubstituted amino acid. In one example, B is an α,α-disubstituted amino acid. For instance, at least one of A, B, C, D or E is 2-aminoisobutyric acid. In other embodiments, at least one of A, B, C, D or E is

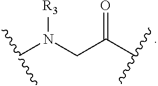

In other embodiments, the length of the macrocycle-forming linker -L₁-L₂- as measured from a first Cα to a second Cα is selected to stabilize a desired secondary peptide structure, such as an α-helix formed by residues of the peptidomimetic macrocycle including, but not necessarily limited to, those between the first Cα to a second Cα.

Exemplary embodiments of the macrocycle-forming linker -L₁-L₂- are shown below.

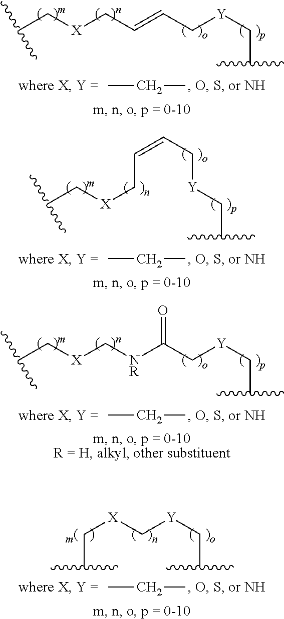

A compound described herein can be at least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure on a chemical, optical, isomeric, enantiomeric, or diastereomeric basis. Purity can be assessed, for example, by HPLC, MS, LC/MS, melting point, or NMR.

### Peptide Homology

Two or more peptides can share a degree of homology. A pair of peptides can have, for example, up to about 20% pairwise homology, up to about 25% pairwise homology, up to about 30% pairwise homology, up to about 35% pairwise homology, up to about 40% pairwise homology, up to about 45% pairwise homology, up to about 50% pairwise homology, up to about 55% pairwise homology, up to about 60% pairwise homology, up to about 65% pairwise homology, up to about 70% pairwise homology, up to about 75% pairwise homology, up to about 80% pairwise homology, up to about 85% pairwise homology, up to about 90% pairwise homology, up to about 95% pairwise homology, up to about 96% pairwise homology, up to about 97% pairwise homology, up to about 98% pairwise homology, up to about 99% pairwise homology, up to about 99.5% pairwise homology, or up to about 99.9% pairwise homology. A pair of peptides can have, for example, at least about 20% pairwise homology, at least about 25% pairwise homology, at least about 30% pairwise homology, at least about 35% pairwise homology, at least about 40% pairwise homology, at least about 45% pairwise homology, at least about 50% pairwise homology, at least about 55% pairwise homology, at least about 60% pairwise homology, at least about 65% pairwise homology, at least about 70% pairwise homology, at least about 75% pairwise homology, at least about 80% pairwise homology, at least about 85% pairwise homology, at least about 90% pairwise homology, at least about 95% pairwise homology, at least about 96% pairwise homology, at least about 97% pairwise homology, at least about 98% pairwise homology, at least about 99% pairwise homology, at least about 99.5% pairwise homology, at least about 99.9% pairwise homology.

Various methods and software programs can be used to determine the homology between two or more peptides, such as NCBI BLAST, Clustal W, MAFFT, Clustal Omega, AlignMe, Praline, or another suitable method or algorithm.

### Preparation of Peptidomimetic Macrocycles

Peptidomimetic macrocycles may be prepared by any of a variety of methods known in the art. For example, any of the residues indicated by “X”, “Z” or “XX” in Table 1a, 1b, 2a, 2b, or 2c may be substituted with a residue capable of forming a crosslinker with a second residue in the same molecule or a precursor of such a residue.

Various methods to effect formation of peptidomimetic macrocycles are known in the art. For example, the preparation of peptidomimetic macrocycles of Formula (I) is described in Schafmeister et al., J. Am. Chem. Soc. 122:5891-5892 (2000); Schafmeister & Verdine, J. Am. Chem. Soc. 122:5891 (2005); Walensky et al., Science 305:1466-1470 (2004); U.S. Pat. No. 7,192,713 and PCT application WO 2008/121767. The α,α-disubstituted amino acids and amino acid precursors disclosed in the cited references may be employed in synthesis of the peptidomimetic macrocycle precursor polypeptides. For example, the “S5-olefin amino acid” is (S)-α-(2′-pentenyl) alanine and the “R₈ olefin amino acid” is (R)-α-(2′-octenyl) alanine. Following incorporation of such amino acids into precursor polypeptides, the terminal olefins are reacted with a metathesis catalyst, leading to the formation of the peptidomimetic macrocycle. In various embodiments, the following amino acids may be employed in the synthesis of the peptidomimetic macrocycle:

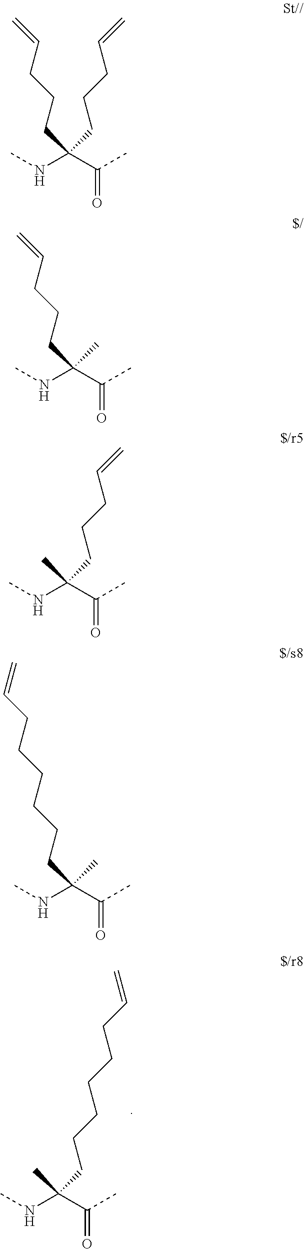

In various embodiments, the following amino acids may be employed in the synthesis of the peptidomimetic macrocycle, wherein L′ is an atom (for example, C, O, N, or S); and g is an integer from 1-20, for example, 1, 2, 3, 4, 5, 6, 7, 8, 9, 10, 11, 12, 13, 14, 15, 16, 17, 18, 19, or 20:

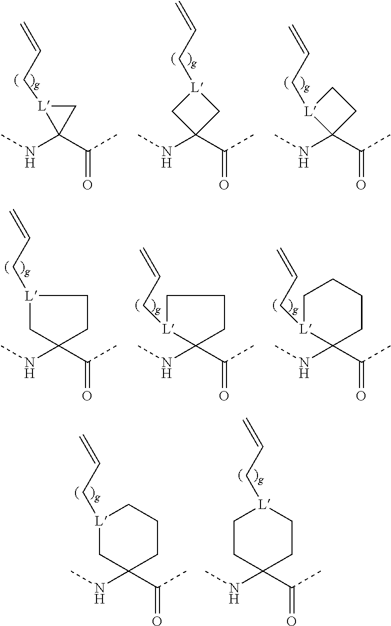

In some embodiments, the invention provides a method for synthesizing a peptidomimetic macrocycle, the method comprising the steps of contacting a peptidomimetic precursor of Formula (V) or Formula (VI):

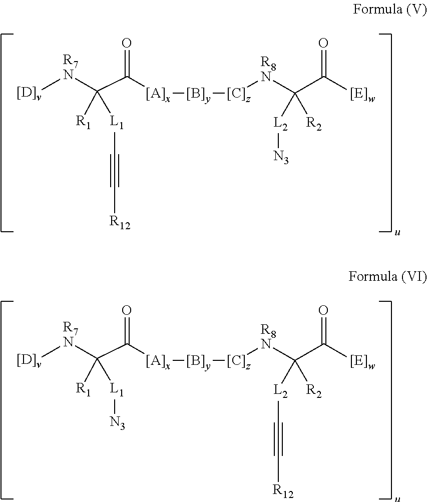

with a macrocyclization catalyst;

wherein v, w, x, y, z, A, B, C, D, E, R₁, R₂, R₇, R₈, L₁ and L₂ are as defined for Formula (II); R₁₂ is —H when the macrocyclization catalyst is a Cu catalyst and R₁₂ is —H or alkyl when the macrocyclization catalyst is a Ru catalyst, and further wherein the contacting step results in a covalent linkage being formed between the alkyne and azide moiety in Formula (V) or Formula (VI). For example, R₁₂ may be methyl when the macrocyclization catalyst is a Ru catalyst.

In the peptidomimetic macrocycles, at least one of R₁ and R₂ is alkyl, alkenyl, alkynyl, arylalkyl, cycloalkyl, cycloalkylalkyl, heteroalkyl, or heterocycloalkyl, unsubstituted or substituted with halo-. In some embodiments, both R₁ and R₂ are independently alkyl, alkenyl, alkynyl, arylalkyl, cycloalkyl, cycloalkylalkyl, heteroalkyl, or heterocycloalkyl, unsubstituted or substituted with halo-. In some embodiments, at least one of A, B, C, D or E is an α,α-disubstituted amino acid. In one example, B is an α,α-disubstituted amino acid. For instance, at least one of A, B, C, D or E is 2-aminoisobutyric acid.

For example, at least one of R₁ and R₂ is alkyl, unsubstituted or substituted with halo-. In another example, both R₁ and R₂ are independently alkyl, unsubstituted or substituted with halo-. In some embodiments, at least one of R₁ and R₂ is methyl. In other embodiments, R₁ and R₂ are methyl. The macrocyclization catalyst may be a Cu catalyst or a Ru catalyst.

In some embodiments, the peptidomimetic precursor is purified prior to the contacting step. In other embodiments, the peptidomimetic macrocycle is purified after the contacting step. In still other embodiments, the peptidomimetic macrocycle is refolded after the contacting step. The method may be performed in solution, or, alternatively, the method may be performed on a solid support.

Also envisioned herein is performing the method in the presence of a target macromolecule that binds to the peptidomimetic precursor or peptidomimetic macrocycle under conditions that favor the binding. In some embodiments, the method is performed in the presence of a target macromolecule that binds preferentially to the peptidomimetic precursor or peptidomimetic macrocycle under conditions that favor the binding. The method may also be applied to synthesize a library of peptidomimetic macrocycles.

In some embodiments, the alkyne moiety of the peptidomimetic precursor of Formula (V) or Formula (VI) is a sidechain of an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid, and (R)-2-amino-2-methyl-8-nonynoic acid. In other embodiments, the azide moiety of the peptidomimetic precursor of Formula (V) or Formula (VI) is a sidechain of an amino acid selected from the group consisting of ε-azido-L-lysine, ε-azido-D-lysine, ε-azido-α-methyl-L-lysine, ε-azido-α-methyl-D-lysine, 6-azido-α-methyl-L-ornithine, and 6-azido-α-methyl-D-ornithine.

In some embodiments, x+y+z is 3, and A, B and C are independently natural or non-natural amino acids. In other embodiments, x+y+z is 6, and A, B and C are independently natural or non-natural amino acids.

In some embodiments, the contacting step is performed in a solvent selected from the group consisting of protic solvent, aqueous solvent, organic solvent, and mixtures thereof. For example, the solvent may be chosen from the group consisting of H₂O, THF, THF/H₂O, tBuOH/H₂O, DMF, DIPEA, CH₃CN or CH₂Cl₂, ClCH₂CH₂Cl or a mixture thereof. The solvent may be a solvent which favors helix formation.

Alternative but equivalent protecting groups, leaving groups or reagents are substituted, and certain of the synthetic steps are performed in alternative sequences or orders to produce the desired compounds. Synthetic chemistry transformations and protecting group methodologies (protection and deprotection) useful in synthesizing the compounds described herein include, for example, those such as described in Larock, Comprehensive Organic Transformations, VCH Publishers (1989); Greene and Wuts, Protective Groups in Organic Synthesis, 2d. Ed., John Wiley and Sons (1991); Fieser and Fieser, Fieser and Fieser's Reagents for Organic Synthesis, John Wiley and Sons (1994); and Paquette, ed., Encyclopedia of Reagents for Organic Synthesis, John Wiley and Sons (1995), and subsequent editions thereof.

The peptidomimetic macrocycles disclosed herein are made, for example, by chemical synthesis methods, such as described in Fields et al., Chapter 3 in Synthetic Peptides: A User's Guide, ed. Grant, W. H. Freeman & Co., New York, N.Y., 1992, p. 77. Hence, for example, peptides are synthesized using the automated Merrifield techniques of solid phase synthesis with the amine protected by either tBoc or Fmoc chemistry using side chain protected amino acids on, for example, an automated peptide synthesizer (e.g., Applied Biosystems (Foster City, Calif.), Model 430A, 431, or 433).

One manner of producing the peptidomimetic precursors and peptidomimetic macrocycles described herein uses solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Side chain functional groups are protected as necessary with base stable, acid labile groups.

Longer peptidomimetic precursors are produced, for example, by conjoining individual synthetic peptides using native chemical ligation. Alternatively, the longer synthetic peptides are biosynthesized by well-known recombinant DNA and protein expression techniques. Such techniques are provided in well-known standard manuals with detailed protocols. To construct a gene encoding a peptidomimetic precursor of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptidomimetic precursors are made, for example, in a high-throughput, combinatorial fashion using, for example, a high-throughput polychannel combinatorial synthesizer (e.g., Thuramed TETRAS multichannel peptide synthesizer from CreoSalus, Louisville, Ky. or Model Apex 396 multichannel peptide synthesizer from AAPPTEC, Inc., Louisville, Ky.).

In some embodiments, the peptidomimetic macrocycles comprise triazole macrocycle-forming linkers. For example, the synthesis of such peptidomimetic macrocycles involves a multi-step process that features the synthesis of a peptidomimetic precursor containing an azide moiety and an alkyne moiety; followed by contacting the peptidomimetic precursor with a macrocyclization catalyst to generate a triazole-linked peptidomimetic macrocycle. Such a process is described, for example, in U.S. application Ser. No. 12/037,041, filed on Feb. 25, 2008. Macrocycles or macrocycle precursors are synthesized, for example, by solution phase or solid-phase methods, and can contain both naturally-occurring and non-naturally-occurring amino acids. See, for example, Hunt, “The Non-Protein Amino Acids” in Chemistry and Biochemistry of the Amino Acids, edited by G. C. Barrett, Chapman and Hall, 1985.

In some embodiments, an azide is linked to the α-carbon of a residue and an alkyne is attached to the α-carbon of another residue. In some embodiments, the azide moieties are azido-analogs of amino acids L-lysine, D-lysine, alpha-methyl-L-lysine, alpha-methyl-D-lysine, L-ornithine, D-ornithine, alpha-methyl-L-ornithine or alpha-methyl-D-ornithine. In another embodiment, the alkyne moiety is L-propargylglycine. In yet other embodiments, the alkyne moiety is an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid and (R)-2-amino-2-methyl-8-nonynoic acid.

The following synthetic schemes are provided solely to illustrate the present invention and are not intended to limit the scope of the invention, as described herein. To simplify the drawings, the illustrative schemes depict azido amino acid analogs ε-azido-α-methyl-L-lysine and ε-azido-α-methyl-D-lysine, and alkyne amino acid analogs L-propargylglycine, (S)-2-amino-2-methyl-4-pentynoic acid, and (S)-2-amino-2-methyl-6-heptynoic acid. Thus, in the following synthetic schemes, each R₁, R₂, R₇ and R₈ is —H; each L₁ is —(CH₂)₄—; and each L₂ is —(CH₂)—. However, as noted throughout the detailed description above, many other amino acid analogs can be employed in which R₁, R₂, R₇, R₈, L₁ and L₂ can be independently selected from the various structures disclosed herein.

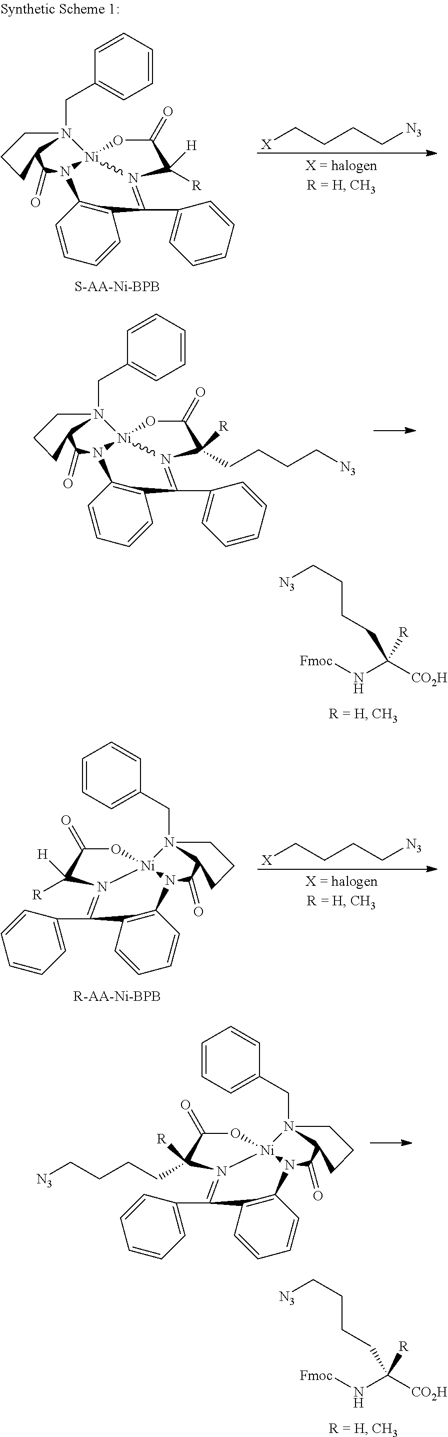

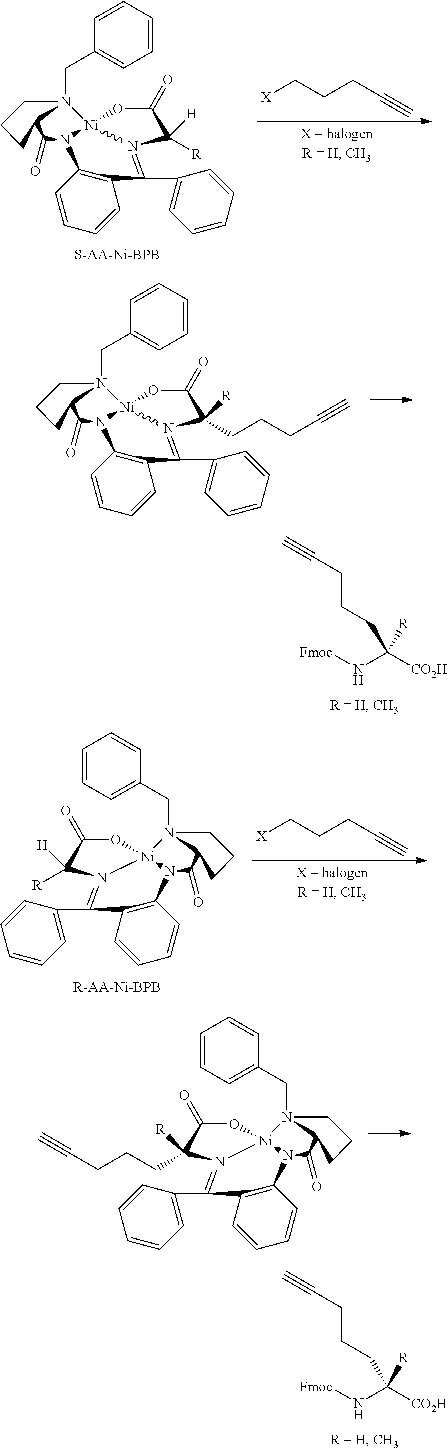

Synthetic Scheme 1 describes the preparation of several compounds of the invention. Ni(II) complexes of Schiff bases derived from the chiral auxiliary (S)-2-[N—(N′-benzylprolyl)amino]benzophenone (BPB) and amino acids such as glycine or alanine are prepared as described in Belokon et al. (1998), Tetrahedron Asymm. 9:4249-4252. The resulting complexes are subsequently reacted with alkylating reagents comprising an azido or alkynyl moiety to yield enantiomerically enriched compounds of the invention. If desired, the resulting compounds can be protected for use in peptide synthesis.

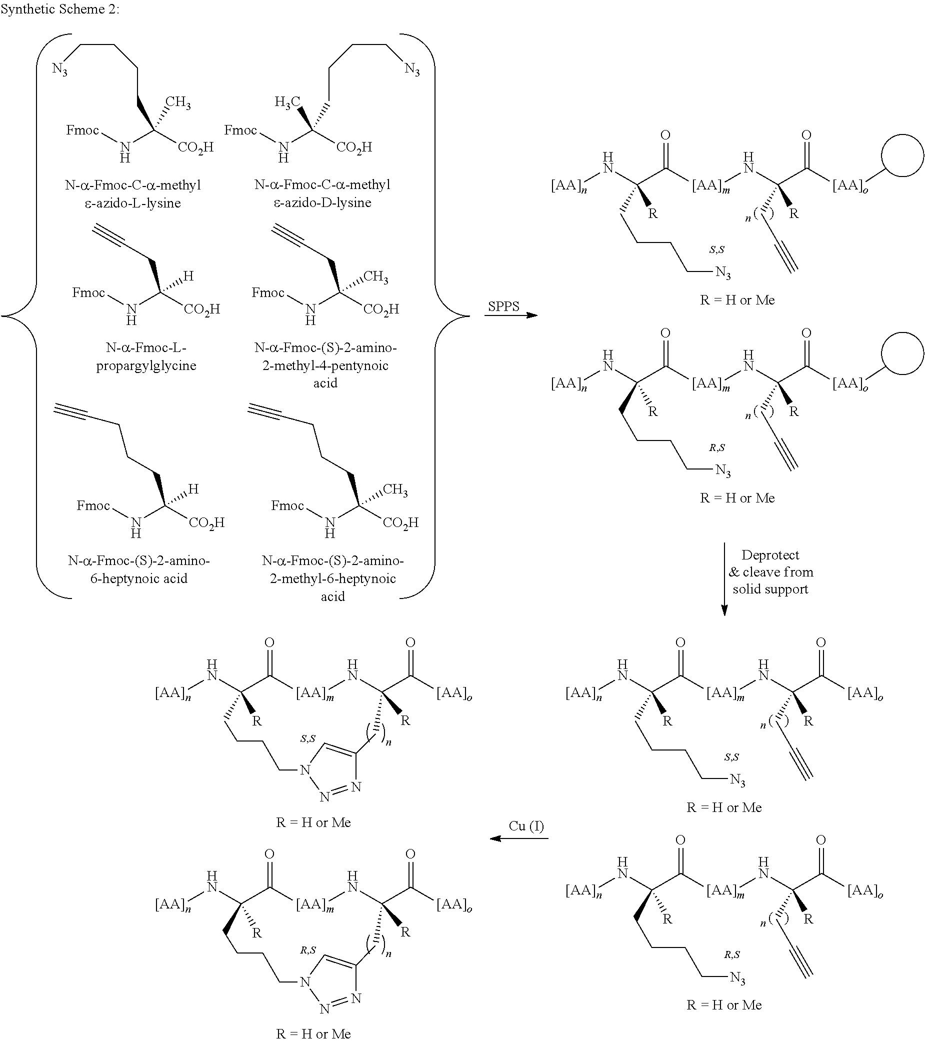

In the general method for the synthesis of peptidomimetic macrocycles shown in Synthetic Scheme 2, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization catalyst such as a Cu(I) in organic or aqueous solutions (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). In one embodiment, the triazole forming reaction is performed under conditions that favor α-helix formation. In one embodiment, the macrocyclization step is performed in a solvent chosen from the group consisting of H₂O, THF, CH₃CN, DMF, DIPEA, tBuOH or a mixture thereof. In another embodiment, the macrocyclization step is performed in DMF. In some embodiments, the macrocyclization step is performed in a buffered aqueous or partially aqueous solvent.

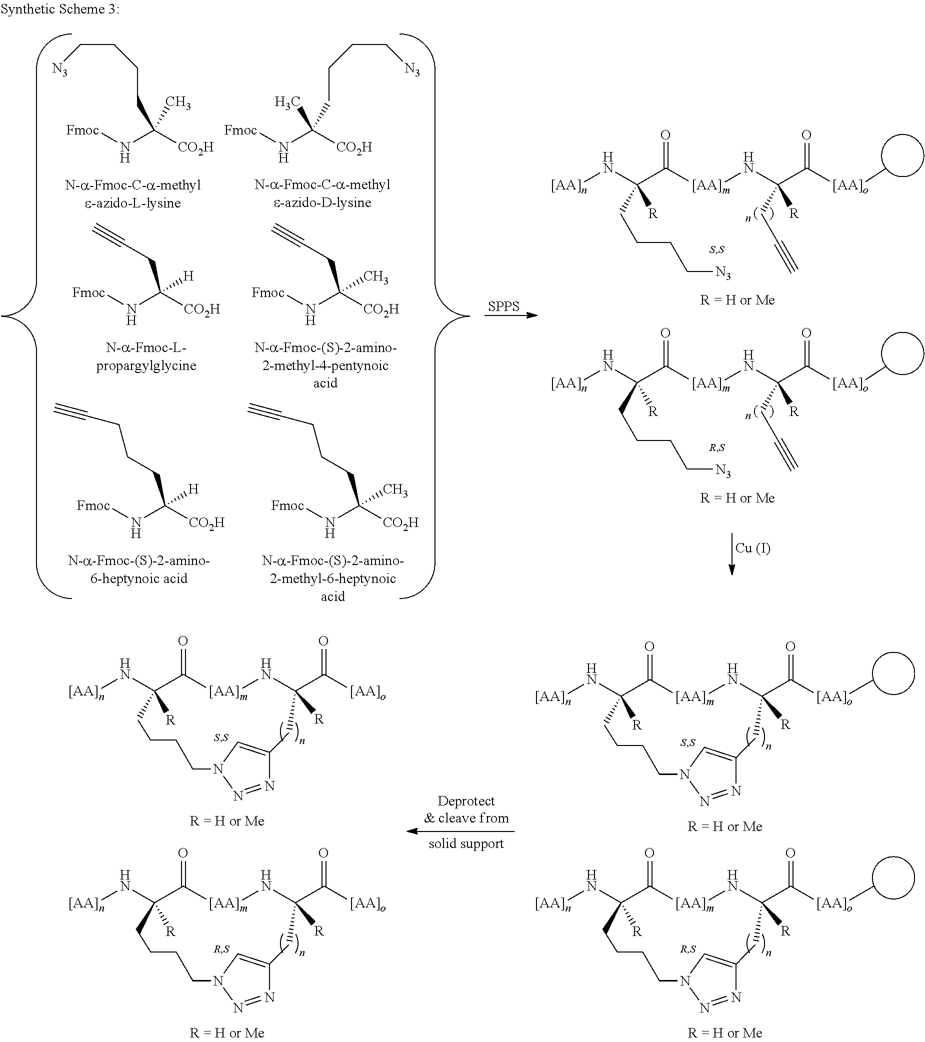

In the general method for the synthesis of peptidomimetic macrocycles shown in Synthetic Scheme 3, the peptidomimetic precursor contains an azide moiety and an alkyne moiety and is synthesized by solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is reacted with a macrocyclization catalyst such as a Cu(I) catalyst on the resin as a crude mixture (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). The resultant triazole-containing peptidomimetic macrocycle is then deprotected and cleaved from the solid-phase resin by standard conditions (e.g., strong acid such as 95% TFA). In some embodiments, the macrocyclization step is performed in a solvent chosen from the group consisting of CH₂Cl₂, ClCH₂CH₂Cl, DMF, THF, NMP, DIPEA, 2,6-lutidine, pyridine, DMSO, H₂O or a mixture thereof. In some embodiments, the macrocyclization step is performed in a buffered aqueous or partially aqueous solvent.

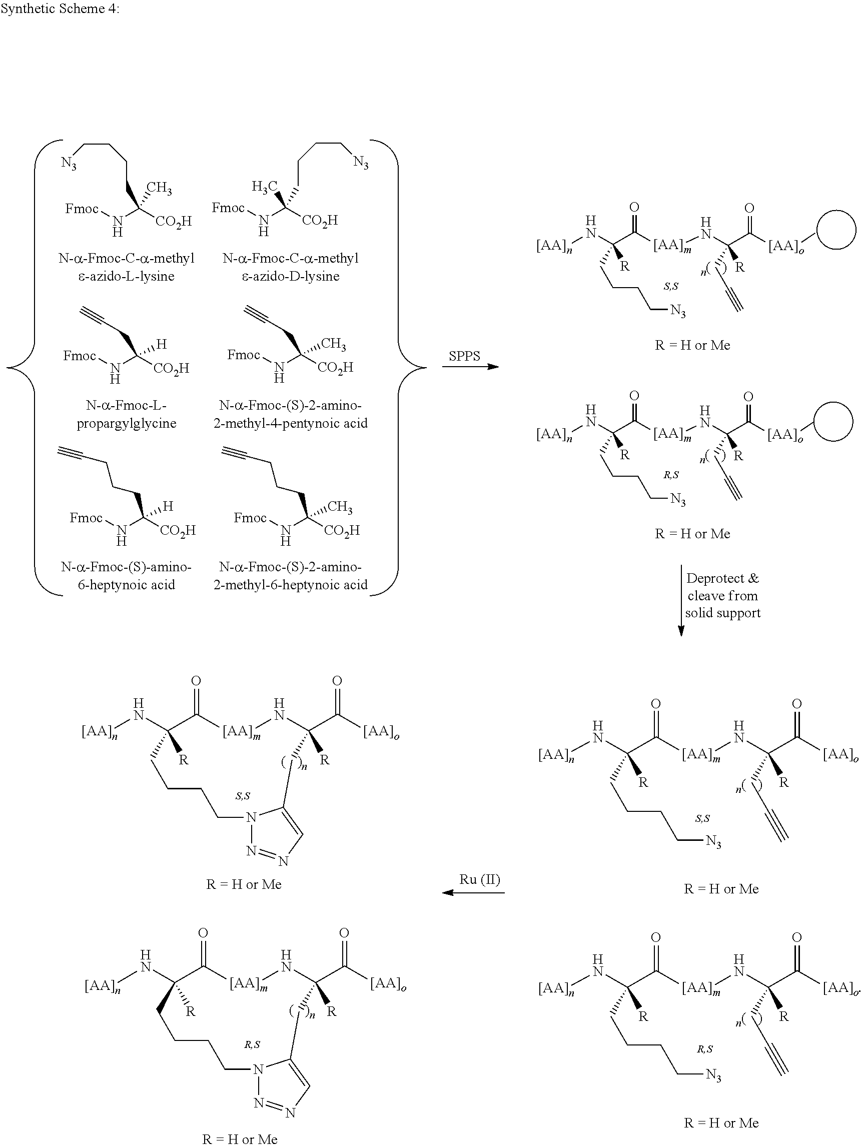

In the general method for the synthesis of peptidomimetic macrocycles shown in Synthetic Scheme 4, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization catalyst such as a Ru(II) catalysts, for example Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of DMF, CH₃CN and THF.

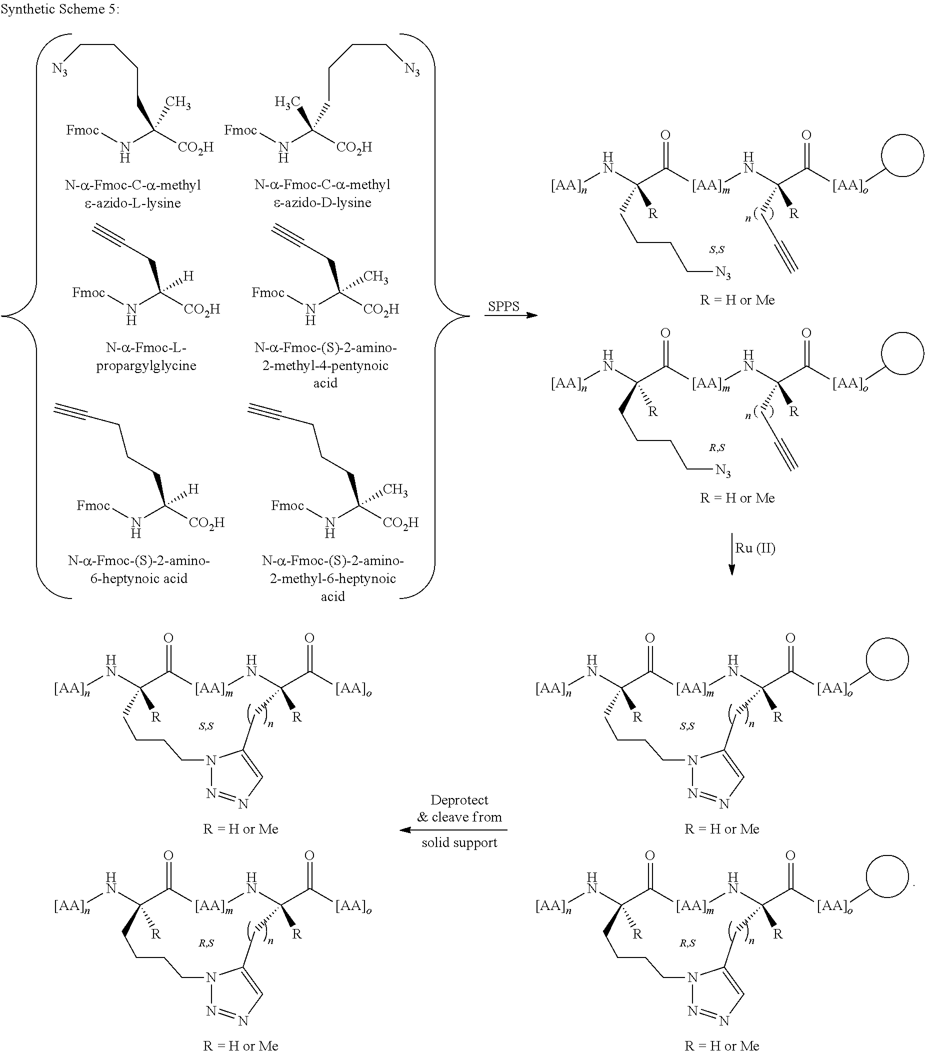

In the general method for the synthesis of peptidomimetic macrocycles shown in Synthetic Scheme 5, the peptidomimetic precursor contains an azide moiety and an alkyne moiety and is synthesized by solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-ε-azido-L-lysine, and N-methyl-ε-azido-D-lysine. The peptidomimetic precursor is reacted with a macrocyclization catalyst such as a Ru(II) catalyst on the resin as a crude mixture. For example, the catalyst can be Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of CH₂Cl₂, ClCH₂CH₂Cl, CH₃CN, DMF, and THF.

The present invention contemplates the use of non-naturally-occurring amino acids and amino acid analogs in the synthesis of the peptidomimetic macrocycles described herein. Any amino acid or amino acid analog amenable to the synthetic methods employed for the synthesis of stable triazole containing peptidomimetic macrocycles can be used. For example, L-propargylglycine is contemplated as a useful amino acid. However, other alkyne-containing amino acids that contain a different amino acid side chain are also useful in the invention, e.g., L-propargylglycine contains one methylene unit between the α-carbon of the amino acid and the alkyne of the amino acid side chain. The invention also contemplates the use of amino acids with multiple methylene units between the α-carbon and the alkyne. Also, the azido-analogs of amino acids L-lysine, D-lysine, alpha-methyl-L-lysine, and alpha-methyl-D-lysine are contemplated as useful amino acids. However, other terminal azide amino acids that contain a different amino acid side chain are also useful in the invention. For example, the azido-analog of L-lysine contains four methylene units between the α-carbon of the amino acid and the terminal azide of the amino acid side chain. The invention also contemplates the use of amino acids with fewer than or greater than four methylene units between the α-carbon and the terminal azide. Table 4 shows some amino acids useful in the preparation of peptidomimetic macrocycles disclosed herein.

TABLE 4

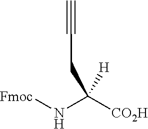

N-α-Fmoc-L-propargyl glycine

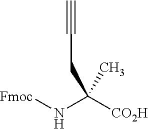

N-α-Fmoc-(S)-2-amino-2-
methyl-4-pentynoic acid

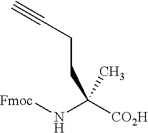

N-α-Fmoc-(S)-2-amino-2-
methyl-5-hexynoic acid

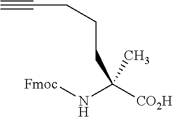

N-α-Fmoc-(S)-2-amino-2-
methyl-6-heptynoic acid

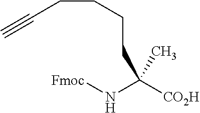

N-α-Fmoc-(S)-2-amino-2-
methyl-7-octynoioc acid

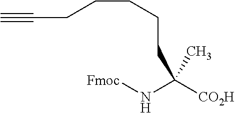

N-α-Fmoc-(S)-2-amino-2-
methyl-8-nonynoic acid

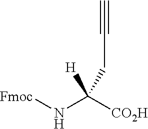

N-α-Fmoc-D-propargyl glycine

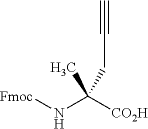

N-α-Fmoc-(R)-2-amino-2-
methyl-4-pentynoic acid

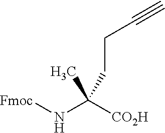

N-α-Fmoc-(R)-2-amino-2-
methyl-5-hexynoic acid

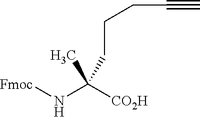

N-α-Fmoc-(R)-2-amino-2-
methyl-6-heptynoic acid

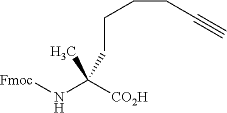

N-α-Fmoc-(R)-2-amino-2-
methyl-7-octynoic acid

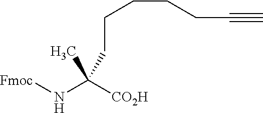

N-α-Fmoc-(R)-2-amino-2-
methyl-8-nonynoic acid

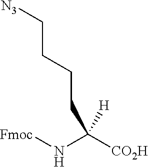

N-α-Fmoc-ε-azido-
L-lysine

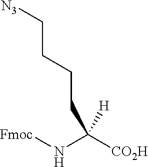

N-α-Fmoc-ε-azido-
α-methyl-L-lysine

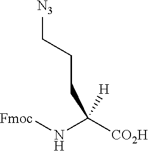

L-α-Fmoc-δ-azido-
L-ornithine

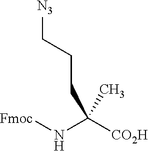

N-α-Fmoc-ε-azido-
α-methyl-L-
ornithine

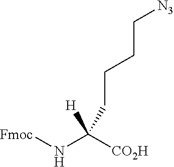

N-α-Fmoc-ε-azido-
D-lysine

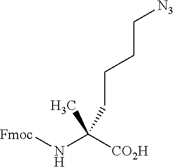

N-α-Fmoc-ε-azido-
α-methyl-D-lysine

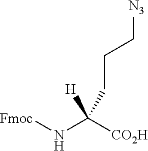

N-α-Fmoc-δ-azido-
D-ornithine

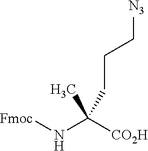

N-α-Fmoc-ε-azido-
α-methyl-D-
ornithine

In some embodiments the amino acids and amino acid analogs are of the D-configuration. In other embodiments they are of the L-configuration. In some embodiments, some of the amino acids and amino acid analogs contained in the peptidomimetic are of the D-configuration while some of the amino acids and amino acid analogs are of the L-configuration. In some embodiments the amino acid analogs are α,α-disubstituted, such as α-methyl-L-propargylglycine, α-methyl-D-propargylglycine, ε-azidRo-α-methyl-L-lysine, and ε-azido-α-methyl-D-lysine. In some embodiments the amino acid analogs are N-alkylated, e.g., N-methyl-L-propargylglycine, N-methyl-D-propargylglycine, N-methyl-α-azido-L-lysine, and N-methyl-ε-azido-D-lysine.

In some embodiments, the —NH moiety of the amino acid is protected using a protecting group, including without limitation -Fmoc and -Boc. In other embodiments, the amino acid is not protected prior to synthesis of the peptidomimetic macrocycle.

In other embodiments, peptidomimetic macrocycles of Formula (III) are synthesized. The preparation of such macrocycles is described, for example, in U.S. application Ser. No. 11/957,325, filed on Dec. 17, 2007. The following synthetic schemes describe the preparation of such compounds. To simplify the drawings, the illustrative schemes depict amino acid analogs derived from L- or D-cysteine, in which L₁ and L₃ are both —(CH₂)—. However, as noted throughout the detailed description above, many other amino acid analogs can be employed in which L₁ and L₃ can be independently selected from the various structures disclosed herein. The symbols “[AA]ₘ”, “[AA],”, “[AA]ₒ” represent a sequence of amide bond-linked moieties such as natural or unnatural amino acids. As described previously, each occurrence of “AA” is independent of any other occurrence of “AA”, and a formula such as “[AA]ₘ” encompasses, for example, sequences of non-identical amino acids as well as sequences of identical amino acids.

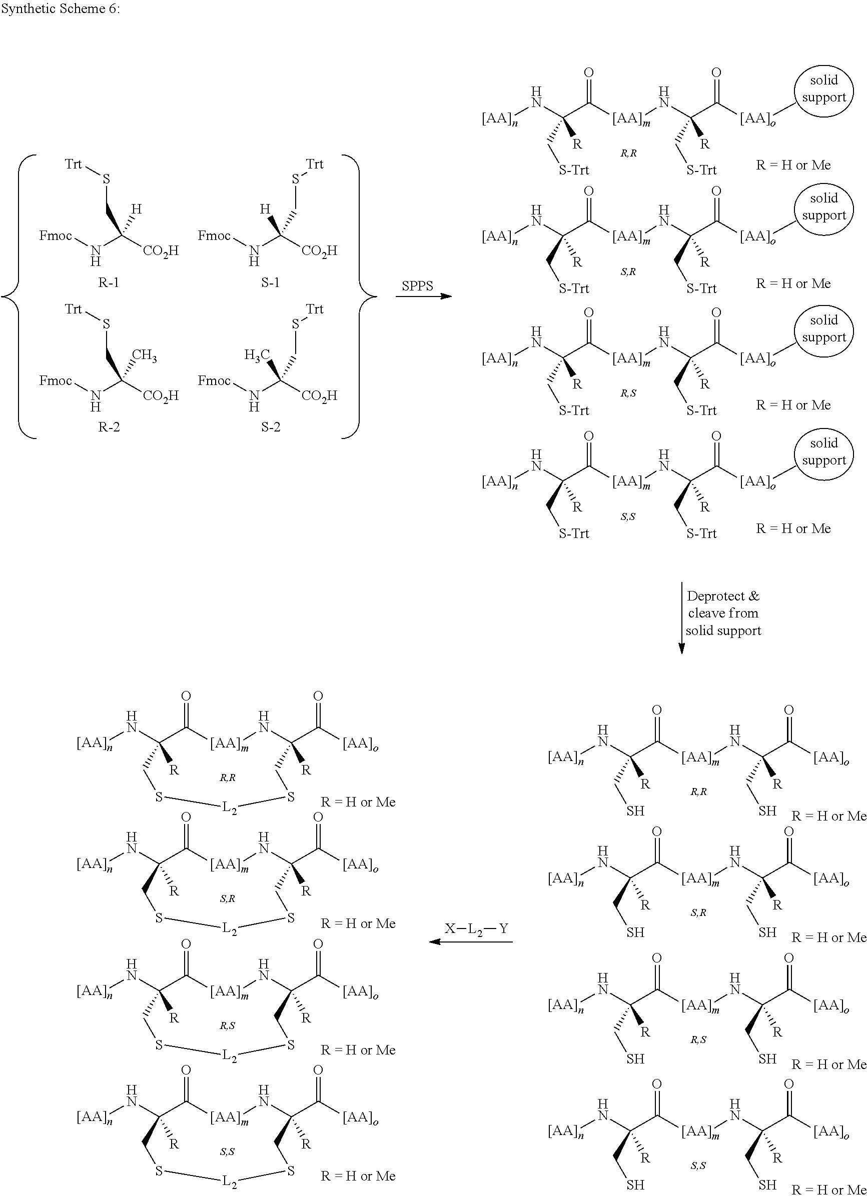

In Scheme 6, the peptidomimetic precursor contains two —SH moieties and is synthesized by solid-phase peptide synthesis (SPPS) using commercially available N-α-Fmoc amino acids such as N-α-Fmoc-S-trityl-L-cysteine or N-α-Fmoc-S-trityl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-trityl monomers by known methods (Bioorganic Chemistry: Peptides and Proteins, Oxford University Press, New York: 1998, the entire contents of which are incorporated herein by reference). The precursor peptidomimetic is then deprotected and cleaved from the solid-phase resin by standard conditions (e.g., strong acid such as 95% TFA). The precursor peptidomimetic is reacted as a crude mixture or is purified prior to reaction with X-L₂-Y in organic or aqueous solutions. In some embodiments the alkylation reaction is performed under dilute conditions (i.e. 0.15 mmol/L) to favor macrocyclization and to avoid polymerization. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH, or NH₃/DMF (Or et al. (1991), J. Org. Chem. 56:3146-3149). In other embodiments, the alkylation is performed in an aqueous solution such as 6M guanidinium HCL, pH 8 (Brunel et al. (2005), Chem. Commun. (20):2552-2554). In other embodiments, the solvent used for the alkylation reaction is DMF or dichloroethane.

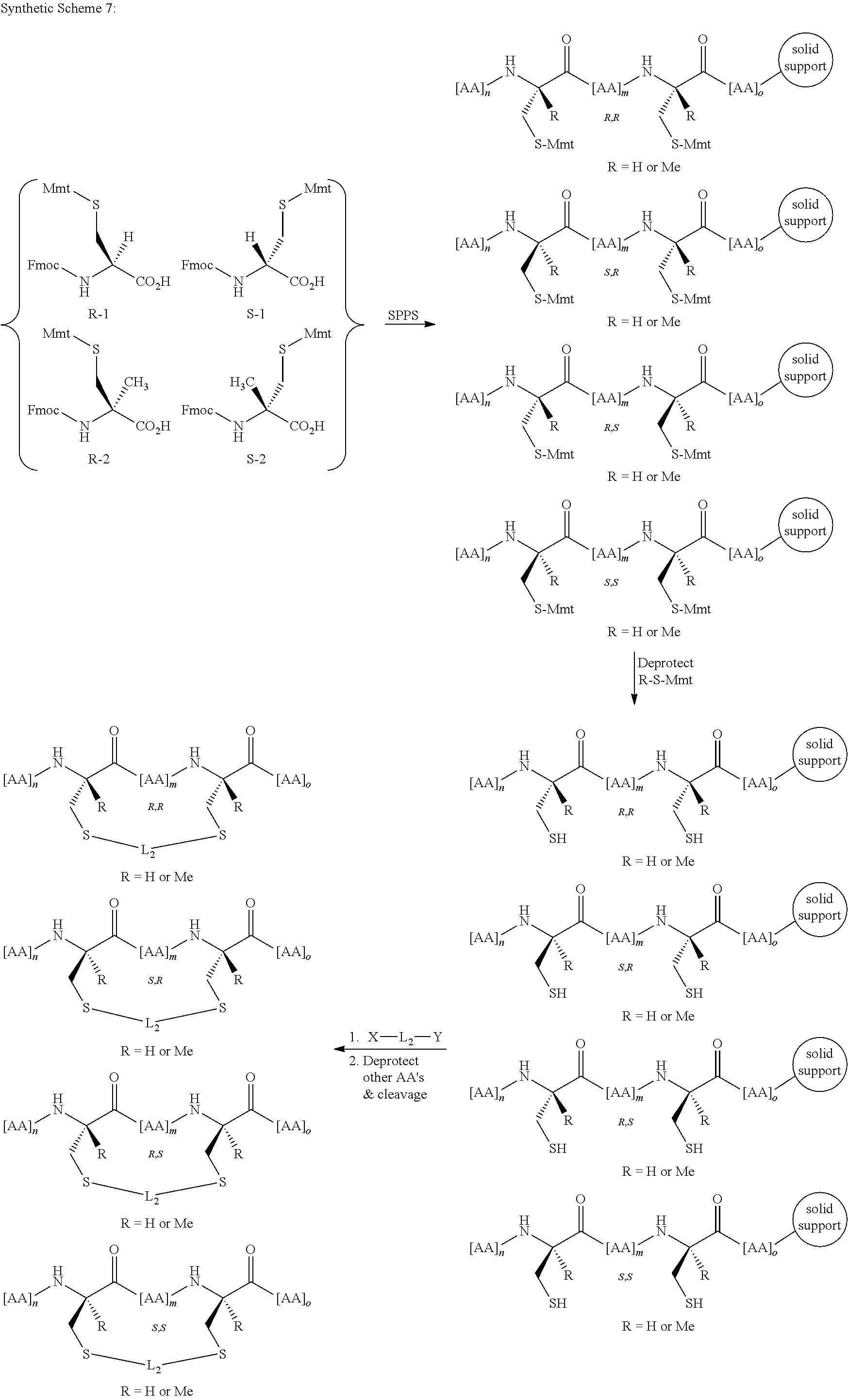

In Scheme 7, the precursor peptidomimetic contains two or more —SH moieties, of which two are specially protected to allow their selective deprotection and subsequent alkylation for macrocycle formation.

The precursor peptidomimetic is synthesized by solid-phase peptide synthesis (SPPS) using commercially available N-α-Fmoc amino acids such as N-α-Fmoc-S-p-methoxytrityl-L-cysteine or N-α-Fmoc-S-p-methoxytrityl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-p-methoxytrityl monomers by known methods (Bioorganic Chemistry: Peptides and Proteins, Oxford University Press, New York: 1998, the entire contents of which are incorporated herein by reference). The Mmt protecting groups of the peptidomimetic precursor are then selectively cleaved by standard conditions (e.g., mild acid such as 1% TFA in DCM). The precursor peptidomimetic is then reacted on the resin with X-L₂-Y in an organic solution. For example, the reaction takes place in the presence of a hindered base such as diisopropylethylamine. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH or NH₃/DMF (Or et al. (1991), J. Org. Chem. 56:3146-3149). In other embodiments, the alkylation reaction is performed in DMF or dichloroethane. The peptidomimetic macrocycle is then deprotected and cleaved from the solid-phase resin by standard conditions (e.g., strong acid such as 95% TFA).

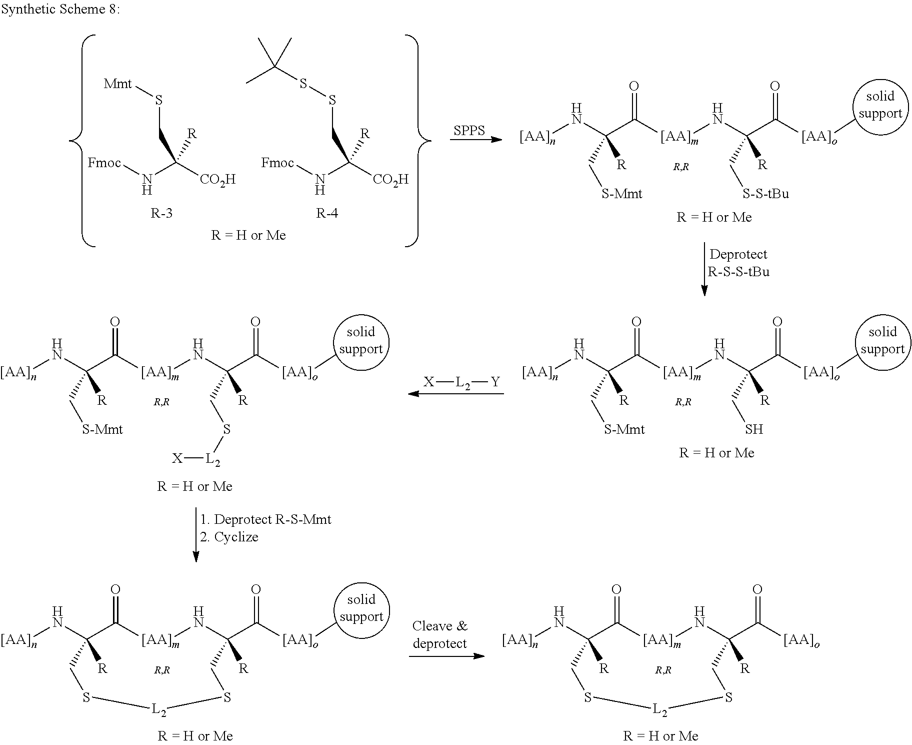

In Scheme 8, the peptidomimetic precursor contains two or more —SH moieties, of which two are specially protected to allow their selective deprotection and subsequent alkylation for macrocycle formation. The peptidomimetic precursor is synthesized by solid-phase peptide synthesis (SPPS) using commercially available N-α-Fmoc amino acids such as N-α-Fmoc-S-p-methoxytrityl-L-cysteine, N-α-Fmoc-S-p-methoxytrityl-D-cysteine, N-α-Fmoc-S—S-t-butyl-L-cysteine, and N-α-Fmoc-S—S-t-butyl-D-cysteine. Alpha-methylated versions of D-cysteine or L-cysteine are generated by known methods (Seebach et al. (1996), Angew. Chem. Int. Ed. Engl. 35:2708-2748, and references therein) and then converted to the appropriately protected N-α-Fmoc-S-p-methoxytrityl or N-α-Fmoc-S—S-t-butyl monomers by known methods (Bioorganic Chemistry: Peptides and Proteins, Oxford University Press, New York: 1998, the entire contents of which are incorporated herein by reference). The S—S-tButyl protecting group of the peptidomimetic precursor is selectively cleaved by known conditions (e.g., 20% 2-mercaptoethanol in DMF, reference: Galande et al. (2005), J. Comb. Chem. 7:174-177). The precursor peptidomimetic is then reacted on the resin with a molar excess of X-L₂-Y in an organic solution. For example, the reaction takes place in the presence of a hindered base such as diisopropylethylamine. The Mmt protecting group of the peptidomimetic precursor is then selectively cleaved by standard conditions (e.g., mild acid such as 1% TFA in DCM). The peptidomimetic precursor is then cyclized on the resin by treatment with a hindered base in organic solutions. In some embodiments, the alkylation reaction is performed in organic solutions such as NH₃/MeOH or NH₃/DMF (Or et al. (1991), J. Org. Chem. 56:3146-3149). The peptidomimetic macrocycle is then deprotected and cleaved from the solid-phase resin by standard conditions (e.g., strong acid such as 95% TFA).

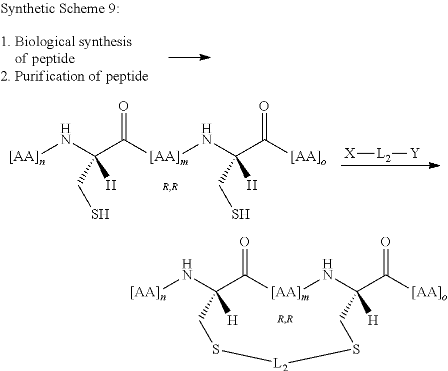

In Scheme 9, the peptidomimetic precursor contains two L-cysteine moieties. The peptidomimetic precursor is synthesized by known biological expression systems in living cells or by known in vitro, cell-free, expression methods. The precursor peptidomimetic is reacted as a crude mixture or is purified prior to reaction with X-L2-Y in organic or aqueous solutions. In some embodiments the alkylation reaction is performed under dilute conditions (i.e. 0.15 mmol/L) to favor macrocyclization and to avoid polymerization. In some embodiments, the alkylation reaction is performed in organic solutions such as liquid NH₃ (Mosberg et al. (1985), J. Am. Chem. Soc. 107:2986-2987; Szewczuk et al. (1992), Int. J. Peptide Protein Res. 40:233-242), NH₃/MeOH, or NH₃/DMF (Or et al. (1991), J. Org. Chem. 56:3146-3149). In other embodiments, the alkylation is performed in an aqueous solution such as 6M guanidinium HCL, pH 8 (Brunel et al. (2005), Chem. Commun. (20):2552-2554). In other embodiments, the alkylation is performed in DMF or dichloroethane. In another embodiment, the alkylation is performed in non-denaturing aqueous solutions, and in yet another embodiment the alkylation is performed under conditions that favor helical structure formation. In yet another embodiment, the alkylation is performed under conditions that favor the binding of the precursor peptidomimetic to another protein, so as to induce the formation of the bound helical conformation during the alkylation.

Various embodiments for X and Y are envisioned which are suitable for reacting with thiol groups. In general, each X or Y is independently be selected from the general category shown in Table 5. For example, X and Y are halides such as —Cl, —Br or —I. Any of the macrocycle-forming linkers described herein may be used in any combination with any of the sequences shown in Table 1a, 1b 2a 2b or 2c and also with any of the R-substituents indicated herein.

TABLE 5
Table 5: Examples of Reactive Groups Capable of
Reacting with Thiol Groups and Resulting Linkages

| X or Y | Resulting Covalent Linkage |
|---|---|
| acrylamide | Thioether |
| halide (e.g., alkyl or aryl halide) | Thioether |
| sulfonate | Thioether |
| aziridine | Thioether |
| epoxide | Thioether |
| haloacetamide | Thioether |
| maleimide | Thioether |
| sulfonate ester | Thioether |

The present invention contemplates the use of both naturally-occurring and non-naturally-occurring amino acids and amino acid analogs in the synthesis of the peptidomimetic macrocycles of Formula (III). Any amino acid or amino acid analog amenable to the synthetic methods employed for the synthesis of stable bis-sulfhydryl containing peptidomimetic macrocycles can be used. For example, cysteine is contemplated as a useful amino acid. However, sulfur containing amino acids other than cysteine that contain a different amino acid side chain are also useful. For example, cysteine contains one methylene unit between the α-carbon of the amino acid and the terminal —SH of the amino acid side chain. The invention also contemplates the use of amino acids with multiple methylene units between the α-carbon and the terminal —SH. Non-limiting examples include α-methyl-L-homocysteine and α-methyl-D-homocysteine. In some embodiments the amino acids and amino acid analogs are of the D-configuration. In other embodiments they are of the L-configuration. In some embodiments, some of the amino acids and amino acid analogs contained in the peptidomimetic are of the D-configuration while some of the amino acids and amino acid analogs are of the L-configuration. In some embodiments the amino acid analogs are α,α-disubstituted, such as α-methyl-L-cysteine and α-methyl-D-cysteine.

The invention includes macrocycles in which macrocycle-forming linkers are used to link two or more —SH moieties in the peptidomimetic precursors to form the peptidomimetic macrocycles. As described above, the macrocycle-forming linkers impart conformational rigidity, and/or increased metabolic stability. Furthermore, in some embodiments, the macrocycle-forming linkages stabilize a helical secondary structure of the peptidomimetic macrocycles. The macrocycle-forming linkers are of the formula X-L₂-Y, wherein both X and Y are the same or different moieties, as defined above. Both X and Y have the chemical characteristics that allow one macrocycle-forming linker -L₂- to bis alkylate the bis-sulfhydryl containing peptidomimetic precursor. As defined above, the linker -L₂- includes alkylene, alkenylene, alkynylene, heteroalkylene, cycloalkylene, heterocycloalkylene, arylene, or heteroarylene, or —R₄—K—R₄—, all of which can be optionally substituted with an R₅ group, as defined above. Furthermore, one to three carbon atoms within the macrocycle-forming linkers -L₂-, other than the carbons attached to the —SH of the sulfhydryl containing amino acid, are optionally substituted with a heteroatom such as N, S or O.

The L₂ component of the macrocycle-forming linker X-L₂-Y may be varied in length depending on, among other things, the distance between the positions of the two amino acid analogs used to form the peptidomimetic macrocycle. Furthermore, as the lengths of L₁ and/or L₃ components of the macrocycle-forming linker are varied, the length of L₂ can also be varied in order to create a linker of appropriate overall length for forming a stable peptidomimetic macrocycle. For example, if the amino acid analogs used are varied by adding an additional methylene unit to each of L₁ and L₃, the length of L₂ are decreased in length by the equivalent of approximately two methylene units to compensate for the increased lengths of L₁ and L₃.

In some embodiments, L₂ is an alkylene group of the formula —(CH₂)ₙ—, where n is an integer between about 1 and about 15. For example, n is 1, 2, 3, 4, 5, 6, 7, 8, 9 or 10. In other embodiments, L₂ is an alkenylene group. In still other embodiments, L₂ is an aryl group.

Table 6 shows additional embodiments of X-L₂-Y groups. Each X and Y in Table 6 is, for example, independently Cl—, Br— or I—.

TABLE 6
Table 6: Exemplary X—L₂—Y groups of the invention.

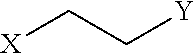

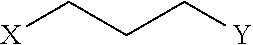

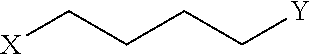

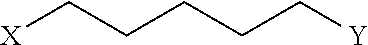

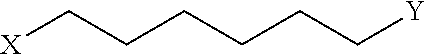

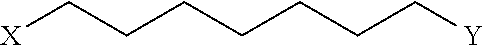

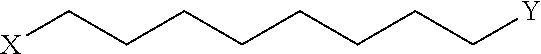

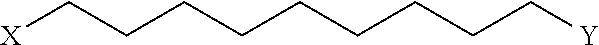

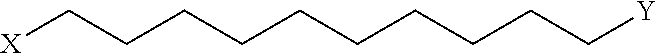

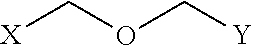

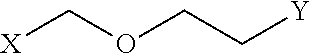

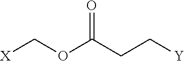

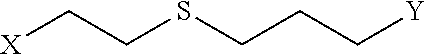

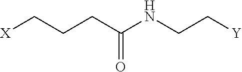

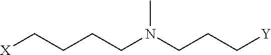

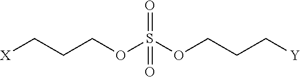

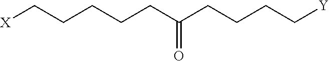

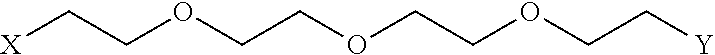

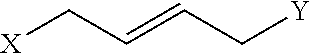

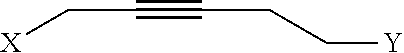

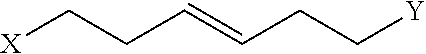

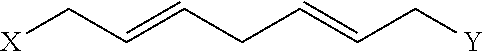

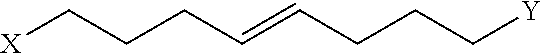

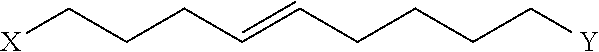

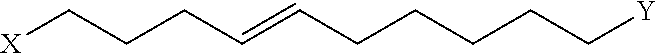

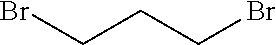

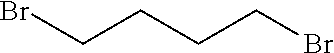

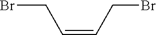

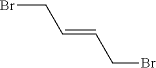

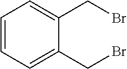

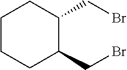

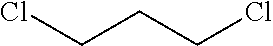

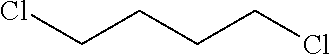

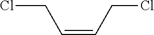

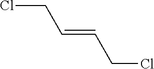

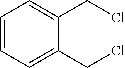

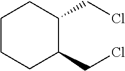

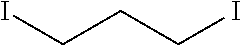

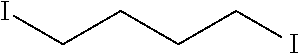

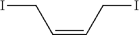

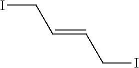

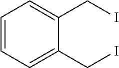

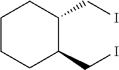

Additional methods of forming peptidomimetic macrocycles which are envisioned as suitable to perform the present invention include those disclosed by Mustapa, M. Firouz Mohd et al., J. Org. Chem (2003), 68, pp. 8193-8198; Yang, Bin et al. Bioorg Med. Chem. Lett. (2004), 14, pp. 1403-1406; U.S. Pat. No. 5,364,851; U.S. Pat. No. 5,446,128; U.S. Pat. No. 5,824,483; U.S. Pat. No. 6,713,280; and U.S. Pat. No. 7,202,332. In such embodiments, amino acid precursors are used containing an additional substituent R— at the alpha position. Such amino acids are incorporated into the macrocycle precursor at the desired positions, which may be at the positions where the crosslinker is substituted or, alternatively, elsewhere in the sequence of the macrocycle precursor. Cyclization of the precursor is then performed according to the indicated method.

For example, a peptidomimetic macrocycle of Formula (II) is prepared as indicated:

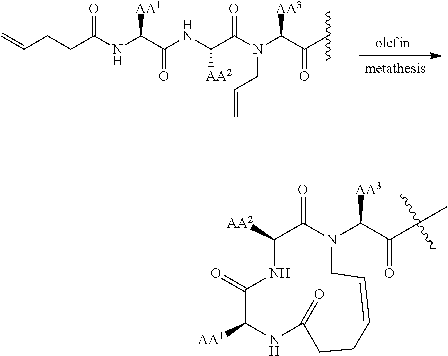

wherein each AA₁, AA₂, AA₃ is independently an amino acid side chain.

In other embodiments, a peptidomimetic macrocycle of Formula (II) is prepared as indicated:

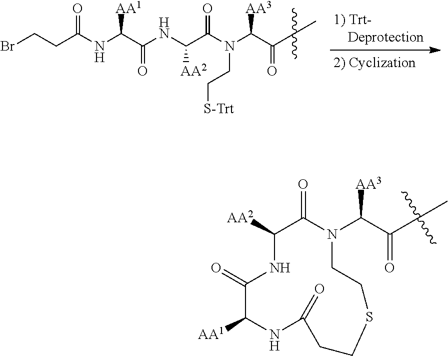

wherein each AA₁, AA₂, AA₃ is independently an amino acid side chain.

In some embodiments, a peptidomimetic macrocycle is obtained in more than one isomer, for example due to the configuration of a double bond within the structure of the crosslinker (E vs Z). Such isomers can or cannot be separable by conventional chromatographic methods. In some embodiments, one isomer has improved biological properties relative to the other isomer. In one embodiment, an E crosslinker olefin isomer of a peptidomimetic macrocycle has better solubility, better target affinity, better in vivo or in vitro efficacy, or higher helicity relative to its Z counterpart. In another embodiment, a Z crosslinker olefin isomer of a peptidomimetic macrocycle has better solubility, better target affinity, better in vivo or in vitro efficacy, or higher helicity relative to its E counterpart.

### Assays

The properties of the peptidomimetic macrocycles are assayed, for example, by using the methods described below. In some embodiments, a peptidomimetic macrocycle has improved biological properties relative to a corresponding polypeptide lacking the substituents described herein.

### Assay to Determine α-Helicity.

In solution, the secondary structure of polypeptides with α-helical domains will reach a dynamic equilibrium between random coil structures and α-helical structures, often expressed as a “percent helicity”.

Thus, for example, alpha-helical domains are predominantly random coils in solution, with α-helical content usually under 25%. Peptidomimetic macrocycles with optimized linkers, on the other hand, possess, for example, an alpha-helicity that is at least two-fold greater than that of a corresponding uncrosslinked polypeptide. In some embodiments, macrocycles will possess an alpha-helicity of greater than 50%. To assay the helicity of peptidomimetic macrocycles of the invention, the compounds are dissolved in an aqueous solution (e.g., 50 mM potassium phosphate solution at pH 7, or distilled H₂O, to concentrations of 25-50 μM). Circular dichroism (CD) spectra are obtained on a spectropolarimeter (e.g., Jasco J-710) using standard measurement parameters (e.g., temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity (e.g., [Φ]222obs) by the reported value for a model helical decapeptide (Yang et al. (1986), Methods Enzymol. 130:208)).

### Assay to Determine Melting Temperature (Tm).

A peptidomimetic macrocycle comprising a secondary structure such as an α-helix exhibits, for example, a higher melting temperature than a corresponding uncrosslinked polypeptide. Typically peptidomimetic macrocycles exhibit Tm of >60° C. representing a highly stable structure in aqueous solutions.

To assay the effect of macrocycle formation on melting temperature, peptidomimetic macrocycles or unmodified peptides are dissolved in distilled H₂O (e.g., at a final concentration of 50 μM) and the Tm is determined by measuring the change in ellipticity over a temperature range (e.g., 4 to 95° C.) on a spectropolarimeter (e.g., Jasco J-710) using standard parameters (e.g., wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

### Protease Resistance Assay.

The amide bond of the peptide backbone is susceptible to hydrolysis by proteases, thereby rendering peptidic compounds vulnerable to rapid degradation in vivo. Peptide helix formation, however, typically buries the amide backbone and therefore may shield it from proteolytic cleavage. The peptidomimetic macrocycles of the present invention may be subjected to in vitro trypsin proteolysis to assess for any change in degradation rate compared to a corresponding uncrosslinked polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln [S] versus time (k=−1×slope).

### Ex Vivo Stability Assay.

Peptidomimetic macrocycles with optimized linkers possess, for example, an ex vivo half-life that is at least two-fold greater than that of a corresponding uncrosslinked polypeptide, and possess an ex vivo half-life of 12 hours or more. For ex vivo serum stability studies, a variety of assays may be used. For example, a peptidomimetic macrocycle and a corresponding uncrosslinked polypeptide (2 mcg) are incubated with fresh mouse, rat and/or human serum (2 mL) at 37° C. for 0, 1, 2, 4, 8, and 24 hours. To determine the level of intact compound, the following procedure may be used: The samples are extracted by transferring 100 μl of sera to 2 ml centrifuge tubes followed by the addition of 10 μL of 50% formic acid and 500 μL acetonitrile and centrifugation at 14,000 RPM for 10 min at 4±2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis.

### In Vitro Binding Assays.

To assess the binding and affinity of compounds that antagonize the interaction between a peptide and an acceptor protein, a fluorescence polarization assay (FPA) utilizing a fluoresceinated peptidomimetic macrocycle derived from a peptidomimetic precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g., FITC-labeled peptides bound to a large protein) emit higher levels of polarized fluorescence due to their slower rates of rotation as compared to fluorescent tracers attached to smaller molecules (e.g., FITC-labeled peptides that are free in solution). A compound that antagonizes the interaction between the fluoresceinated peptidomimetic macrocycle and an acceptor protein will be detected in a competitive binding FPA experiment.

For example, putative antagonist compounds (1 nM to 1 mM) and a fluoresceinated peptidomimetic macrocycle (25 nM) are incubated with the acceptor protein (50 nM) in binding buffer (140 mM NaCl, 50 mM Tris-HCL, pH 7.4) for 30 minutes at room temperature. Antagonist binding activity is measured, for example, by fluorescence polarization on a luminescence spectrophotometer (e.g., Perkin-Elmer LS50B). Kd values may be determined by nonlinear regression analysis using, for example, Graphpad Prism software (GraphPad Software, Inc., San Diego, Calif.).

Any class of molecule, such as small organic molecules, peptides, oligonucleotides or proteins can be examined as putative antagonists in this assay.

### In Vitro Activity Assay

DiscoverX cAmP Hunter eXpress VIPRI CHO-K1 GPCR and GHRHR CHO-K1 GPCR assays kits were used. The DiscoverX kits contain naturally coupled GPCR cell lines designed to detect GPCR signaling through second messenger activation. This signaling involves a membrane bound enzyme called adenylate cyclase. G1- and G2- coupled receptors modulate cAMP by either inhibiting or stimulating adenylate cyclase, respectively. The DiscoverX cell lines included in the kits utilize the natural coupling status of the GPCR to monitor activation of G1- and G2- coupled receptors. Following ligand stimulation, the functional status of the GPCR is monitored by measuring cellular cAMP levels using a homogeneous, gain of signal competitive immunoassay based on Enzyme Fragment Complementation (EFC). The amount of EFC is measured by analysis with a fluorescent plate reader.

On the day before the assay, the DiscoverX cell lines were defrosted, plated into 384 well plates and allowed to incubate overnight. All the samples were diluted to 2 mM using 100% DMSO. The vials were sonicated and centrifuged to assure all peptides went into solution. The final volumes were small, 80-200 μl. An 80% purity and peptide content was assumed for all samples and dilutions were based on the molecular weights. On the day of the assay, the samples were diluted to 200 μM in water and then to 4 μM in DiscoverX assay buffer (supplemented with 0.1% BSA). Serial dilutions were performed on assay day, 16 dilutions from 1000 nM were run side by side in duplicate for each peptide for study. The assays were performed in the 384 well plates with the assistance of a CyBio Bi-Well 384 channel liquid handling robot. The samples were analyzed on a Tecan Ultra Evolution plate reader according to DiscoverX guidelines. The EC₅₀ values were determined using GraphPad Prism software. The EC₅₀ value is defined as the concentration of agonist that provokes a response halfway between the baseline and maximum response.

### In Vivo Stability Assay.

To investigate the in vivo stability of the peptidomimetic macrocycles, the compounds are, for example, administered to mice and/or rats by IV, IP, PO or inhalation routes at concentrations ranging from 0.1 to 50 mg/kg and blood specimens withdrawn at 0′, 5′, 15′, 30′, 1 hr, 4 hrs, 8 hrs and 24 hours post-injection. Levels of intact compound in 25 μL of fresh serum are then measured by LC-MS/MS as above.

### Clinical Trials.

To determine the suitability of the peptidomimetic macrocycles for treatment of humans, clinical trials are performed. For example, patients diagnosed with a muscle wasting disease or lipodystrophy and in need of treatment are selected and separated in treatment and one or more control groups, wherein the treatment group is administered a peptidomimetic macrocycle of the invention, while the control groups receive a placebo or a known GHRH or GH drug. The treatment safety and efficacy of the peptidomimetic macrocycles can thus be evaluated by performing comparisons of the patient groups with respect to factors such as survival and quality-of-life. In this example, the patient group treated with a peptidomimetic macrocycle show improved long-term survival compared to a patient control group treated with a placebo.

### Pharmaceutical Compositions and Routes of Administration

The peptidomimetic macrocycles also include pharmaceutically acceptable derivatives or prodrugs thereof. A “pharmaceutically acceptable derivative” means any pharmaceutically acceptable salt, ester, salt of an ester, pro-drug or other derivative of a compound of this invention which, upon administration to a recipient, is capable of providing (directly or indirectly) a compound of this invention. Particularly favored pharmaceutically acceptable derivatives are those that increase the bioavailability of the compounds when administered to a mammal (e.g., by increasing absorption into the blood of an orally administered compound) or which increases delivery of the active compound to a biological compartment (e.g., the brain or lymphatic system) relative to the parent species. Some pharmaceutically acceptable derivatives include a chemical group which increases aqueous solubility or active transport across the gastrointestinal mucosa.

In some embodiments, the peptidomimetic macrocycles are modified by covalently or non-covalently joining appropriate functional groups to enhance selective biological properties. Such modifications include those which increase biological penetration into a given biological compartment (e.g., blood, lymphatic system, central nervous system), increase oral availability, increase solubility to allow subcutaneous administration or administration by injection, alter metabolism, and alter rate of excretion. In some embodiments, a peptidomimetic macrocycle or pharmaceutically acceptable salt thereof is not precipitated in the formulation. In some embodiments, a peptidomimetic macrocycle or pharmaceutically acceptable salt thereof comprising a PEG functional group is not precipitated in the formulation. In some embodiments, a peptidomimetic macrocycle or pharmaceutically acceptable salt thereof comprising a PEG functional group is has a 2, 3, 4, 5, 6, 7, 8, 9, 10, 20, 30, 40, 50, 60, 70, 80, 90, or 100 or more fold increase in solubility compared to respective a peptidomimetic macrocycle or pharmaceutically acceptable salt thereof not comprising the PEG functional group.

In some embodiments, the peptidomimetic macrocycles are formulated in an aqueous solution. In some embodiments, the peptidomimetic macrocycles are formulated in a biological liquid, such as plasma. In some embodiments, the peptidomimetic macrocycles are soluble in an aqueous solution or in a biological liquid, such as plasma. For example, the peptidomimetic macrocycles can have a solubility in an aqueous solution or in a biological liquid, such as plasma, that is at least about 1 mg/mL, 1.5 mg/mL, 1.6 mg/mL, 1.7 mg/mL, 1.8 mg/mL, 1.9 mg/mL, 2 mg/mL, 2.5 mg/mL, 3 mg/mL, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, 10 mg/mL, 10.5 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, or higher. For example, the peptidomimetic macrocycles can have a solubility in an aqueous solution or in a biological liquid, such as plasma, that is from about 1-200 mg/mL, 1-150 mg/mL, 1-100 mg/mL, 1-75 mg/mL, 1-50 mg/mL, 1-25 mg/mL, 1-20 mg/mL, 1-15 mg/mL, 1-10 mg/mL, 1-5 mg/mL, 5-200 mg/mL, 5-150 mg/mL, 5-100 mg/mL, 5-75 mg/mL, 5-50 mg/mL, 5-25 mg/mL, 5-20 mg/mL, 5-15 mg/mL, 5-10 mg/mL, 10-200 mg/mL, 10-150 mg/mL, 10-100 mg/mL, 10-75 mg/mL, 10-50 mg/mL, 10-25 mg/mL, 10-20 mg/mL, 10-15 mg/mL, 10-200 mg/mL, 20-150 mg/mL, 20-100 mg/mL, 20-75 mg/mL, 20-50 mg/mL, 20-25 mg/mL, 50-200 mg/mL, 50-150 mg/mL, 50-100 mg/mL, 50-75 mg/mL, 75-200 mg/mL, 75-150 mg/mL, or 75-100 mg/mL.

In some embodiments, peptidomimetic macrocycles comprising a PEG moiety have a solubility in an aqueous solution or in a biological liquid, such as plasma, that is at least about 1.1, 1.2, 1.3, 1.4, 1.5 1.6, 1.7, 1.8, 1.9, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1000 times higher than the solubility of a corresponding peptidomimetic macrocycle that does not comprises the PEG moiety.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄₊ salts.

For preparing pharmaceutical compositions from the compounds of the present invention, pharmaceutically acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the cross-linked polyvinyl pyrrolidone, agar, alginic acid, or a salt thereof, such as sodium alginate.

Liquid form preparations include solutions, suspensions, and emulsions, for example, water or water/propylene glycol solutions. For parenteral injection, liquid preparations can be formulated in solution in aqueous polyethylene glycol solution.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

When the compositions of this invention comprise a combination of a peptidomimetic macrocycle and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the additional agents are administered separately, as part of a multiple dose regimen, from the compounds of this invention. Alternatively, those agents are part of a single dosage form, mixed together with the compounds of this invention in a single composition.

In some embodiments, the compositions are present as unit dosage forms that can deliver, for example, from about 0.0001 mg to about 1,000 mg of the peptidomimetic macrocycles, salts thereof, prodrugs thereof, derivatives thereof, or any combination of these. Thus, the unit dosage forms can deliver, for example, in some embodiments, from about 1 mg to about 900 mg, from about 1 mg to about 800 mg, from about 1 mg to about 700 mg, from about 1 mg to about 600 mg, from about 1 mg to about 500 mg, from about 1 mg to about 400 mg, from about 1 mg to about 300 mg, from about 1 mg to about 200 mg, from about 1 mg to about 100 mg, from about 1 mg to about 10 mg, from about 1 mg to about 5 mg, from about 0.1 mg to about 10 mg, from about 0.1 mg to about 5 mg, from about 10 mg to about 1,000 mg, from about 50 mg to about 1,000 mg, from about 100 mg to about 1,000 mg, from about 200 mg to about 1,000 mg, from about 300 mg to about 1,000 mg, from about 400 mg to about 1,000 mg, from about 500 mg to about 1,000 mg, from about 600 mg to about 1,000 mg, from about 700 mg to about 1,000 mg, from about 800 mg to about 1,000 mg, from about 900 mg to about 1,000 mg, from about 10 mg to about 900 mg, from about 100 mg to about 800 mg, from about 200 mg to about 700 mg, or from about 300 mg to about 600 mg of the peptidomimetic macrocycles, salts thereof, prodrugs thereof, derivatives thereof, or any combination of these.

In some embodiments, the compositions are present as unit dosage forms that can deliver, for example, about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 500 mg, about 600 mg, about 700 mg, about 800 mg, or about 800 mg of peptidomimetic macrocycles, salts thereof, prodrugs thereof, derivatives thereof, or any combination of these.

Suitable routes of administration include, but are not limited to, oral, intravenous, rectal, aerosol, parenteral, ophthalmic, pulmonary, transmucosal, transdermal, vaginal, otic, nasal, and topical administration.

In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a composition as described herein is administered in a local rather than systemic manner, for example, via injection of the compound directly into an organ. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In another embodiment, compositions described herein are formulated for oral administration. Compositions described herein are formulated by combining a peptidomimetic macrocycle with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the peptidomimetic macrocycles described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form.

The sugar solutions optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the peptidomimetic macrocycles described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the peptidomimetic macrocycles described herein are formulated for buccal or sublingual administration. Formulations suitable for buccal or sublingual administration include, by way of example only, tablets, lozenges, or gels. In still other embodiments, the peptidomimetic macrocycles described herein are formulated for parenteral injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, pharmaceutical compositions are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions herein can be administered, for example, once or twice or three or four or five or six times per day, or once or twice or three or four or five or six times per week, and can be administered, for example, for a day, a week, a month, 3 months, six months, a year, five years, or for example ten years.

### Methods of Use

In one aspect, the present invention provides novel peptidomimetic macrocycles that are useful in competitive binding assays to identify agents which bind to the natural ligand(s) of the proteins or peptides upon which the peptidomimetic macrocycles are modeled. For example, in the GHRH system, labeled peptidomimetic macrocycles based on GHRH can be used in a binding assay along with small molecules that competitively bind to the GHRH receptor. Competitive binding studies allow for rapid in vitro evaluation and determination of drug candidates specific for the GHRH system. Such binding studies may be performed with any of the peptidomimetic macrocycles disclosed herein and their binding partners.

The invention further provides for the generation of antibodies against the peptidomimetic macrocycles. In some embodiments, these antibodies specifically bind both the peptidomimetic macrocycle and the precursor peptides, such as GHRH, to which the peptidomimetic macrocycles are related. Such antibodies, for example, disrupt the native protein-protein interactions, for example, between GHRH and the GHRH receptor.

In another aspect, the present invention provides methods to activate the GHRH receptor, thereby stimulating production and release of growth hormone, which in turn can increase lean muscle mass or reduce adipose tissue, for example visceral and/or abdominal adipose tissue. In some embodiments, subject suffering from obesity, for example abdominal obesity, are treated using pharmaceutical compositions of the invention. See, e.g., Makimura et al., J. Clin. Endocrinol. Metab. 2009, 94(12): 5131-5138, which is hereby incorporated by reference.

In yet another aspect, the present invention provides methods for treating muscle wasting diseases that include anorexias, cachexias (such as cancer cachexia, chronic heart failure cachexia, chronic obstructive pulmonary disease cachexia, rheumatoid arthritis cachexia, cachexia in liver cirrohsis) and sarcopenias, methods for treating lipodystrophies that include HIV lipodystrophy, methods for treating growth hormone disorders that include adult and pediatric growth hormone deficiencies, or methods for treating gastroparesis or short bowel syndrome. These methods comprise administering an effective amount of a compound to a warm blooded animal, including a human. In some embodiments, a pharmaceutical composition provided herein used in the treatment of muscle wasting diseases is administered no more frequently than once daily, no more frequently than every other day, no more frequently than twice weekly, no more frequently than weekly, or no more frequently than every other week.

In some embodiments, provided herein are methods for treating adult growth hormone deficiencies. Such deficiencies may be cause, for example, by damage or injury to the pituitary gland or the hypothalamus. Frequently, adult-onset growth hormone deficiency is caused by pituitary tumors or treatment of such tumors, for example by cranial irradiation. Adult growth hormone deficiency may also be caused by a reduced blood supply to the pituitary gland. In some embodiments, a pharmaceutical composition used in treatment of adult growth hormone deficiency is administered no more frequently than once daily, no more frequently than every other day, no more frequently than twice weekly, no more frequently than weekly, or no more frequently than every other week.

In some embodiments, provided herein are methods for treating pediatric growth hormone deficiencies. Growth hormone deficiency in children is often idiopathic. However, possible causes include mutations in genes including GHRHR or GH1, congenital malformations involving the pituitary (such as septo-optic dysplasia or posterior pituitary ectopia), chronic kidney disease, intracranial tumors (e.g., in or near the sella turcica, such as craniopharyngioma), damage to the pituitary from radiation therapy to the cranium (for cancers such as leukemia or brain tumors), surgery, trauma or intracranial disease (e.g., hydrocephalus), autoimmune inflammation (hypophysitis), ischemic or hemorrhagic infarction from low blood pressure (Sheehan syndrome) or hemorrhage pituitary apoplexy. Growth hormone deficiency is observed in congenital diseases such as Prader-Willi syndrome, Turner syndrome, or short stature homeobox gene (SHOX) deficiency, idiopathic short stature, or in infants who are small for gestational age. In some embodiments, a composition used in treatment of pediatric growth hormone deficiency is administered no more frequently than once daily, no more frequently than every other day, no more frequently than twice weekly, no more frequently than weekly, or no more frequently than every other week.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope and that methods and structures within the scope of these claims and their equivalents be covered thereby.

In the sequences shown above and elsewhere, the following abbreviations are used: amino acids represented as “$” are alpha-Me S5-pentenyl-alanine olefin amino acids connected by an all-carbon i to i+4 crosslinker comprising one double bond. “%” are alpha-Me S5-pentenyl-alanine olefin amino acids connected by an all-carbon i to i+4 crosslinker comprising no double bonds (fully saturated alkylene crosslinker). Amino acids represented as “$r8” are alpha-Me R₈-octenyl-alanine olefin amino acids connected by an all-carbon i to i+7 crosslinker comprising one double bond. Amino acids represented as “% r8” are alpha-Me R₈-octenyl-alanine olefin amino acids connected by an all-carbon i to i+7 crosslinker comprising no double bonds (fully saturated alkylene crosslinker). The designation “iso1” or “iso2” indicates that the peptidomimetic macrocycle is a single isomer. Amino acids designated as lower case “a” represent D-Alanine.

Amino acids which are used in the formation of triazole crosslinkers are represented according to the legend indicated below. Stereochemistry at the alpha position of each amino acid is S unless otherwise indicated. For azide amino acids, the number of carbon atoms indicated refers to the number of methylene units between the alpha carbon and the terminal azide. For alkyne amino acids, the number of carbon atoms indicated is the number of methylene units between the alpha position and the triazole moiety plus the two carbon atoms within the triazole group derived from the alkyne.

| | |
|---|---|
| $5a5 | Alpha-Me alkyne 1,5 triazole (5 carbon) |
| $4n3 | Alpha-Me azide 1,5 triazole (3 carbon) |
| $4rn6 | Alpha-Me R-azide 1,4 triazole (6 carbon) |
| $4a5 | Alpha-Me alkyne 1,4 triazole (5 carbon) |

### EXAMPLES

### Example 1

### Peptidomimetic Macrocycles

Peptidomimetic macrocycles were synthesized and purified as previously described and as described below (Schafmeister et al., J. Am. Chem. Soc. 122:5891-5892 (2000); Schafmeister & Verdine, J. Am. Chem. Soc. 122:5891 (2005); Walensky et al., Science 305:1466-1470 (2004); and U.S. Pat. No. 7,192,713). Peptidomimetic macrocycles were designed by replacing two or more naturally occurring amino acids with the corresponding synthetic amino acids. Substitutions were made at i and i+4, and i and i+7 positions. Peptide synthesis was performed either manually or on an automated peptide synthesizer (Applied Biosystems™, model 433A), using solid phase conditions, rink amide AM resin (Novabiochem™), and Fmoc main-chain protecting group chemistry. For the coupling of natural Fmoc-protected amino acids (Novabiochem™), 10 equivalents of amino acid and a 1:1:2 molar ratio of coupling reagents HBTU/HOBt (Novabiochem™)/DIEA were employed. Non-natural amino acids (4 equiv) were coupled with a 1:1:2 molar ratio of HATU (Applied Biosystems)/HOBt/DIEA. The N-termini of the synthetic peptides were acetylated, while the C-termini were amidated.

Purification of cross-linked compounds was achieved by high performance liquid chromatography (HPLC) (Varian™ ProStar) on a reverse phase C18 column (Varian™) to yield the pure compounds. Chemical composition of the pure products was confirmed by LC/MS mass spectrometry (Micromass™ LCT interfaced with Agilent™ 1100 HPLC system) and amino acid analysis (Applied Biosystems™, model 420A) (Table 7).

TABLE 7

| SP | Exact mass | (M + 3)/3 | Found |
|---|---|---|---|
| 6 | 3445.96 | 1149.65 | 1150.27 |
| 7 | 3388.94 | 1130.65 | 1131.30 |
| 8 | 3330.89 | 1111.30 | 1111.96 |
| 9 | 3331.88 | 1111.63 | 1112.23 |
| 10 | 3344.91 | 1115.97 | 1116.58 |
| 11 | 3274.86 | 1092.62 | 1093.27 |
| 12 | 3318.86 | 1107.29 | 1107.98 |
| 13 | 3330.89 | 1111.30 | 1111.96 |
| 14 | 3388.94 | 1130.65 | 1131.30 |
| 15 | 3289.83 | 1097.61 | 1098.35 |
| 16 | 3372.94 | 1125.31 | 1126.02 |
| 17 | 3346.89 | 1116.63 | 1117.32 |
| 18 | 3287.89 | 1096.96 | 1097.61 |
| 19 | 3346.89 | 1116.63 | 1117.32 |
| 20 | 3304.84 | 1102.61 | 1103.35 |
| 21 | 3315.92 | 1106.31 | 1106.96 |
| 22 | 3259.88 | 1087.63 | 1088.36 |
| 23 | 3414.99 | 1139.33 | 1139.99 |
| 24 | 3331.88 | 1111.63 | 1112.33 |
| 25 | 3430.98 | 1144.66 | 1145.36 |
| 26 | 3331.88 | 1111.63 | 1112.33 |
| 27 | 3430.98 | 1144.66 | 1145.36 |
| 28 | 3331.91 | 1111.64 | 1112.33 |
| 29 | 3331.91 | 1111.64 | 1112.33 |
| 30 | 3371.00 | 1124.67 | 1125.37 |
| 31 | 3388.94 | 1130.65 | 1131.30 |
| 32 | 3357.93 | 1120.31 | 1121.02 |
| 33 | 3345.92 | 1116.31 | 1116.95 |
| 34 | 3323.85 | 1108.95 | 1109.37 |
| 35 | 3280.84 | 1094.61 | 1095.12 |
| 36 | 3266.83 | 1089.94 | 1090.49 |
| 37 | 3308.87 | 1103.96 | 1104.55 |
| 38 | 3251.82 | 1084.94 | 1085.22 |
| 39 | 3364.90 | 1122.63 | 1123.25 |
| 40 | 3308.84 | 1103.95 | 1104.55 |
| 41 | 3294.86 | 1099.29 | 1099.84 |
| 42 | 3310.85 | 1104.62 | 1105.20 |
| 43 | 3337.86 | 1113.62 | 1114.18 |
| 44 | 3294.86 | 1099.29 | 1099.56 |
| 45 | 3308.84 | 1103.95 | 1104.55 |
| 46 | 3266.83 | 1089.94 | 1090.49 |
| 47 | 3281.83 | 1094.94 | 1095.49 |
| 48 | 3281.83 | 1094.94 | 1095.49 |
| 49 | 3281.83 | 1094.94 | 1095.49 |
| 50 | 3322.89 | 1108.63 | 1109.18 |
| 51 | 3322.89 | 1108.63 | 1109.27 |
| 52 | 3308.87 | 1103.96 | 1104.55 |
| 53 | 3251.85 | 1084.95 | 1085.49 |
| 54 | 3221.84 | 1074.95 | 1075.50 |
| 55 | 3249.87 | 1084.29 | 1084.85 |
| 56 | 3554.01 | 1185.67 | 1186.35 |
| 57 | 3373.92 | 1125.64 | 1126.30 |
| 58 | 3316.90 | 1106.63 | 1107.24 |
| 59 | 3761.05 | 1254.68 | 1255.29 |
| 60 | 3217.82 | 1073.61 | 1074.30 |
| 61 | 3329.93 | 1110.98 | 1111.68 |
| 62 | 3331.88 | 1111.63 | 1112.33 |
| 63 | 3316.90 | 1106.63 | 1107.33 |
| 64 | 3331.88 | 1111.63 | 1112.23 |
| 65 | 3387.94 | 1130.31 | 1131.02 |
| 66 | 3401.96 | 1134.99 | 1135.64 |
| 67 | 3344.93 | 1115.98 | 1116.68 |
| 68 | 3375.90 | 1126.30 | 1126.95 |
| 69 | 3375.88 | 1126.29 | 1127.13 |
| 70 | 3332.87 | 1111.96 | 1113.16 |
| 121 | 3360.90 | 1121.30 | 1122.41 |
| 122 | 3300.88 | 1101.29 | 1102.15 |
| 123 | 3332.84 | 1111.95 | 1113.16 |
| 124 | 3303.86 | 1102.29 | 1103.17 |
| 125 | 3401.89 | 1134.96 | 1136.11 |
| 126 | 3858.21 | 1287.07 | 1288.23 |
| 127 | 3316.84 | 1106.61 | 1107.79 |
| 128 | 3400.90 | 1134.63 | 1135.83 |
| 129 | 3400.90 | 1134.63 | 1135.83 |
| 130 | 3402.91 | 1135.30 | 1136.20 |
| 131 | 3430.95 | 1144.65 | 1145.55 |
| 132 | 3434.90 | 1145.97 | 1147.12 |
| 133 | 3875.17 | 1292.72 | 1293.97 |
| 134 | 3420.89 | 1141.30 | 1142.40 |
| 135 | 3464.92 | 1155.97 | 1156.83 |
| 136 | 3492.91 | 1165.30 | 1166.46 |
| 137 | 3458.90 | 1153.97 | 1154.80 |
| 138 | 3430.87 | 1144.62 | 1145.73 |
| 90 | 3318.86 | 1107.29 | 1108.53 |
| 104 | 3304.84 | 1102.61 | 1103.44 |
| 105 | 3362.85 | 1121.95 | 1122.69 |
| 106 | 3346.89 | 1116.63 | 1117.42 |
| 107 | 3320.84 | 1107.95 | 1108.72 |
| 108 | 3361.86 | 1121.62 | 1122.41 |
| 109 | 4363.44 | 1455.48 | 1455.81 |
| 110 | 4263.36 | 1422.12 | 1422.96 |
| 111 | 4221.36 | 1408.12 | 1408.62 |
| 112 | 4121.29 | 1374.76 | 1375.49 |
| 113 | 5260.89 | 1754.63 | 1755.63 |
| 114 | 4766.61 | 1589.87 | 1591.01 |
| 115 | 4203.37 | 1402.12 | 1402.61 |
| 116 | 4103.29 | 1368.76 | 1369.48 |
| 117 | 4061.29 | 1354.76 | 1355.13 |
| 118 | 3961.22 | 1321.41 | 1322.10 |
| 119 | 5100.82 | 1701.27 | 1702.24 |
| 120 | 4606.53 | 1536.51 | 1537.25 |

The following protocol was used in the synthesis of dialkyne-crosslinked peptidomimetic macrocycles. Fully protected resin-bound peptides were synthesized on a PEG-PS resin (loading 0.45 mmol/g) on a 0.2 mmol scale. Deprotection of the temporary Fmoc group was achieved by 3×10 min treatments of the resin bound peptide with 20% (v/v) piperidine in DMF. After washing with NMP (3×), dichloromethane (3×) and NMP (3×), coupling of each successive amino acid was achieved with 1×60 min incubation with the appropriate preactivated Fmoc-amino acid derivative. All protected amino acids (0.4 mmol) were dissolved in NMP and activated with HCTU (0.4 mmol) and DIEA (0.8 mmol) prior to transfer of the coupling solution to the deprotected resin-bound peptide. After coupling was completed, the resin was washed in preparation for the next deprotection/coupling cycle. Acetylation of the amino terminus was carried out in the presence of acetic anhydride/DIEA in NMP. The LC-MS analysis of a cleaved and deprotected sample obtained from an aliquot of the fully assembled resin-bound peptide was accomplished in order to verifying the completion of each coupling. In a typical example, tetrahydrofuran (4 ml) and triethylamine (2 ml) were added to the peptide resin (0.2 mmol) in a 40 ml glass vial and shaken for 10 minutes. Pd(PPh₃)₂Cl₂ (0.014 g, 0.02 mmol) and copper iodide (0.008 g, 0.04 mmol) were then added and the resulting reaction mixture was mechanically shaken 16 hours while open to atmosphere. The diyne-cyclized resin-bound peptides were deprotected and cleaved from the solid support by treatment with TFA/H₂O/TIS (95/5/5 v/v) for 2.5 h at room temperature. After filtration of the resin the TFA solution was precipitated in cold diethyl ether and centrifuged to yield the desired product as a solid. The crude product was purified by preparative HPLC.

The following protocol was used in the synthesis of single alkyne-crosslinked peptidomimetic macrocycles. Fully protected resin-bound peptides were synthesized on a Rink amide MBHA resin (loading 0.62 mmol/g) on a 0.1 mmol scale. Deprotection of the temporary Fmoc group was achieved by 2×20 min treatments of the resin bound peptide with 25% (v/v) piperidine in NMP. After extensive flow washing with NMP and dichloromethane, coupling of each successive amino acid was achieved with 1×60 min incubation with the appropriate preactivated Fmoc-amino acid derivative. All protected amino acids (1 mmol) were dissolved in NMP and activated with HCTU (1 mmol) and DIEA (1 mmol) prior to transfer of the coupling solution to the deprotected resin-bound peptide. After coupling was completed, the resin was extensively flow washed in preparation for the next deprotection/coupling cycle. Acetylation of the amino terminus was carried out in the presence of acetic anhydride/DIEA in NMP/NMM. The LC-MS analysis of a cleaved and deprotected sample obtained from an aliquot of the fully assembled resin-bound peptide was accomplished in order to verifying the completion of each coupling. In a typical example, the peptide resin (0.1 mmol) was washed with DCM. Resin was loaded into a microwave vial. The vessel was evacuated and purged with nitrogen. Molybdenumhexacarbonyl (0.01 eq, Sigma Aldrich™ 199959) was added. Anhydrous chlorobenzene was added to the reaction vessel. Then 2-fluorophenol (1 eq, Sigma Aldrich™ F12804) was added. The reaction was then loaded into the microwave and held at 130° C. for 10 minutes. Reaction may need to be pushed a subsequent time for completion. The alkyne metathesized resin-bound peptides were deprotected and cleaved from the solid support by treatment with TFA/H₂O/TIS (94/3/3 v/v) for 3 h at room temperature. After filtration of the resin the TFA solution was precipitated in cold diethyl ether and centrifuged to yield the desired product as a solid. The crude product was purified by preparative HPLC.

### Example 2

### GHRHR Agonism Measured by cAMP

GHRH (1-29) and cross-linked peptidomimetic macrocycles were tested for agonism at the human GHRH receptor (hGHRHR) at various concentrations. Human 293 cells transiently or stably expressing hGHRHR were detached from cell culture flasks with versene (Life Technologies™), suspended in serum-free medium (50 k cells/assay point), and stimulated for 30 min at RT with GHRH (1-29) (Bachem™) or cross-linked peptidomimetic macrocycles. cAMP was quantified using an HTRF®-based assay (CisBio) and used according to the manufacturer's instructions. An EC₅₀ for each agonist was calculated from a non-linear fit of response vs dose (GraphPad™ Prism). The maximum response was determined by stimulating with 10 μM GHRH (1-29). Results are shown in Table 8. (+=>50 nm; ++=>25-50 nm; +++=>10-25 nm; ++++=>1-10 nm; +++++=<1 nm).

TABLE 8

| SP# | GHRH cAMP EC₅₀ nM |
|---|---|
| 1 | ++++ |
| 2 | ++++ |
| 3 | ++++ |
| 4 | +++ |
| 5 | ++++ |
| 6 | ++ |
| 7 | ++++ |
| 8 | + |
| 9 | ++++ |
| 10 | ++++ |
| 11 | +++ |
| 12 | +++++ |
| 13 | +++ |
| 14 | ++++ |
| 15 | +++++ |
| 16 | ++++ |
| 17 | +++ |
| 18 | +++ |
| 19 | ++++ |
| 20 | ++++ |
| 21 | +++ |
| 22 | +++ |
| 23 | ++ |
| 24 | ++++ |
| 25 | ++++ |
| 26 | +++ |
| 27 | ++++ |
| 28 | ++++ |
| 29 | ++++ |
| 30 | ++++ |
| 31 | ++++ |
| 32 | ++++ |
| 33 | +++ |
| 34 | +++++ |
| 35 | +++++ |
| 36 | ++++ |
| 37 | ++++ |
| 38 | ++++ |
| 39 | ++++ |
| 40 | ++++ |
| 41 | +++++ |
| 42 | ++++ |
| 43 | +++++ |
| 44 | ++++ |
| 45 | +++++ |
| 46 | +++++ |
| 47 | +++++ |
| 48 | ++++ |
| 49 | +++++ |
| 50 | +++++ |
| 51 | +++++ |
| 52 | ++++ |
| 53 | ++++ |
| 54 | ++++ |
| 55 | ++++ |
| 56 | + |
| 57 | +++ |
| 58 | + |
| 59 | ++ |
| 60 | ++ |
| 61 | +++ |
| 62 | ++ |
| 63 | +++ |
| 64 | +++ |
| 65 | ++ |
| 66 | ++ |
| 67 | +++ |
| 68 | +++ |

### Example 3

### Plasma PK/PD Study in Rats

Five peptidomimetic macrocycles of the invention, as well as sermorelin, were studied to determine pharmacokinetic and pharmacodynamic parameters in rats. Male Sprague-Dawley rats (300 g, non-fasted, cannulated) were used. The study had three arms: IV administration, SC administration, and SC administration (vehicle control). For experiments using sermorelin, a dose level of 3 mg/kg IV/SC bolus was used (dose volume of 3 mL/kg dose and dose concentration of 1 mg/mL). The vehicle used was: 10 wt % N, N-Dimethylacetamide, 10 wt % DMSO, 2 wt % Solutol HS 15 in water for injection containing 45 mg/mL (4.5 wt %) Mannitol and 25 mM (0.38 wt %) Histidine (pH 7.5; 320 mOsm/kg). The peptide was first dissolved at high concentration in DMA and DMSO before a second dilution in Solutol vehicle.

For experiments using peptidomimetic macrocycles, 0.1 mL of DMA and 0.1 mL of DMSO were used to combine with each mg of macrocycle (˜4.3-4.5 mg of macrocycle used in each experiment). Sonication was used to ensure complete solubilization. 0.8 mL of Solutol vehicle was used for each mg of macrocycle in DMA/DMSO. The solutions were mixed gently with pipet or light vortexing. Fresh vials were used for each day of dosing, and macrocycles were stored solid at −20° C. prior to formulation.

For each study arm, 2 rats were bled (350 μL) at specific time points (5 min, 15 min, 30 min, 1 h, 2 h, 4 h, 8 h, 24 h, and 48 h) and a 150 μL bleed was performed just before dosing. Plasma was prepared into K2EDTA tubes by centrifuging for 20 minutes at 4° C. at 2000 G maximum 30 minutes after collection. From each 350 μL bleed, 120 μL were transferred to one tube for PK studies and 50 μL to another tube for PD studies and frozen immediately. From the 150 μL bleed, 70 μL were transferred to one tube for PD studies and frozen immediately. Results are shown in the table below:

| | AUCinf | AUC Ext (%) | t ½ | MRT | Vdss | Clp |
|---|---|---|---|---|---|---|
| Compound | hr*ng/mL | % | hr | hr | mL/kg | mL/hr/kg |
| SP-1 | 17529 | 13.8 | 55.1 | 1.6 | 267 | 171 |
| SP-6 | 23477 | 16.3 | 20.6 | 3.7 | 474 | 128 |
| SP-8 | 12575 | 4.8 | 10.2 | 1.6 | 390 | 239 |
| SP-21 | 30455 | 9.4 | 10.1 | 5.3 | 524 | 99 |
| SP-32 | 36963 | 3.0 | 9.7 | 2.3 | 190 | 81 |
| Tesamorelin, dog 0.1 μg/kv IV** | 5301 | | 0.4 | | | |
| Tesamorelin, human 0.5, 1, or 2 mg 5C** | | | 2-5 h | | | |

**literature values

### Example 4

### Preparation of Peptidomimetic Macrocycles Using a Boc-Protected Amino Acid

Peptidomimetic macrocycle precursors were prepared as described in Example 1 comprising an R₈ amino acid at position “i” and an S5 amino acid at position “i+7”. The amino acid at position “i+3” was a Boc-protected tryptophan which was incorporated during solid-phase synthesis. Specifically, the Boc-protected tryptophan amino acid shown below (and commercially available, for example, from Novabiochem™) was using during solid phase synthesis:

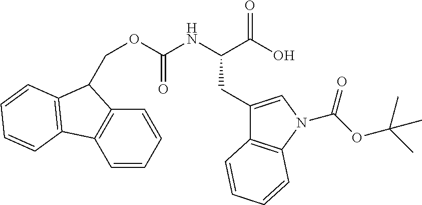 [SMILES: CC(C)(C)OC(=O)n1cc(C[C@H](NC(=O)OCC2c3ccccc3-c3ccccc32)C(=O)O)c2ccccc21]

Metathesis was performed using a ruthenium catalyst prior to the cleavage and deprotection steps. The composition obtained following cyclization was determined by HPLC analysis to contain primarily peptidomimetic macrocycles having a crosslinker comprising a trans olefin (“iso2”, comprising the double bond in an E configuration). Unexpectedly, a ratio of 90:10 was observed for the trans and cis products, respectively.

### Example 5

### In Vitro cAMP Activity Assay to Measure GHRHR Agonism

DiscoverX™ cAmP Hunter eXpress VIPR1 CHO-K1 GPCR and GHRHR CHO-K1 GPCR assay kits were used. On the day before the assay, the DiscoverX™ cell lines were defrosted, plated into 384 well plates and allowed to incubate overnight. All the samples were diluted to 2 mM using 100% DMSO. The vials were sonicated and centrifuged to assure all peptides went into solution. The final volumes were small, 80-200 μl. An 80% purity and peptide content was assumed for all samples and dilutions were based on the molecular weights. On the day of the assay, the samples were diluted to 200 μM in water and then to 4 μM in DiscoverX™ assay buffer (supplemented with 0.1% BSA). Serial dilutions were performed on assay day, 16 dilutions from 1000 nM were run side by side in duplicate for each peptide for study. The assays were performed in the 384 well plates with the assistance of a CyBio™ Bi-Well 384 channel liquid handling robot. The samples were analyzed on a Tecan™ Ultra Evolution plate reader according to DiscoverX™ guidelines. The EC₅₀ values were determined using GraphPad™ Prism software. The EC₅₀ value is defined as the concentration of agonist that provokes a response halfway between the baseline and maximum response. (+=>25 nM; ++=>10-25 nM; +++=>1-10 nM; ++++=>0.1-1 nM; +++++=<0.1 nM). The selectivity value is the concentration of agonist that provokes a response halfway between the baseline and maximum response in VIPR1 CHO-K1 cells divided by the concentration of agonist that provokes a response halfway between the baseline and maximum response in GHRHR CHO-K1 cells. (+=<5; ++=>5-15; +++=>15-25; ++++=>25-50; +++++=>50).

TABLE 9

| SP# | GHRHR (EC₅₀) nM | VIPR1* (EC₅₀) nM | Selectivity |
|---|---|---|---|
| 2 | +++++ | ++++ | ++++ |
| Sermorelin | | | |
| (GRF 1-29)) | | | |
| 3 | ++++ | ++ | +++++ |
| 12 | +++++ | +++++ | + |
| 69 | ++++ | ++++ | + |
| 135 | +++++ | ++++ | +++ |
| 133 | +++ | + | ++ |

*Vasointestinal Peptide Receptor 1

### Example 6

### Determination of Solubility of Peptidomimetic Macrocycles by Measuring Turbidity

Increasing amounts of SP-3 and SP-133 were added to human plasma. The OD₆₀₀ of each of the solutions was then measured. Results can be seen in Table 10. SP-133 showed no turbidity up to 100 mg/mL.

TABLE 10

| SP# | mg/mL | OD₆₀₀ |
|---|---|---|
| 3 | 100 | 1.5 |
| | 77 | 1.7 |
| | 59 | 1.8 |
| | 46 | 1.7 |
| | 35 | 1.6 |
| | 27 | 1.6 |
| | 21 | 1.4 |
| | 16 | 1.2 |
| | 12 | 1.2 |
| | 10 | 1.0 |
| | 7 | 0.8 |
| 133 | 100 | 0.0 |
| | 77 | 0.0 |
| | 59 | 0.0 |
| | 46 | 0.0 |
| | 35 | 0.0 |
| | 27 | 0.0 |
| | 21 | 0.0 |
| | 16 | 0.0 |
| | 12 | 0.0 |
| | 10 | 0.0 |
| | 7 | 0.0 |

The plasma compatibility of exemplary peptidomimetic macrocycles was also determined. Results can be seen in Table 11. Peptidomimetic macrocycles with a plasma compatibility value (PC) that is greater than 0.1 were determined as being plasma compatible.

TABLE 11

| | Plasma Compatability |
|---|---|
| SP# | (mg/mL) |
| 1 (Tesamorelin) | 3.5 |
| 2 (Sermorelin (GRF 1-29)) | 4.6 |
| 3 | 0.9 |
| 12 | 1.6 |
| 69 | 1.6 |
| 135 | 1.6 |
| 133 | >10 |
| 109 | >10 |
| 110 | >10 |
| 111 | >10 |
| 112 | >10 |
| 113 | >10 |
| 114 | >10 |
| 115 | >10 |
| 116 | >10 |
| 117 | 1.2 |
| 118 | 0.9 |
| 119 | 1.2 |
| 120 | 0.7 |
| 104 | 1.2 |
| 105 | 1.2 |
| 106 | 0.9 |
| 107 | 1.6 |
| 108 | 1.6 |

1. A peptidomimetic macrocycle or a pharmaceutically-acceptable salt thereof comprising an amino acid sequence which is at least about 60% identical to GHRH 1-29, and a macrocycle-forming linker connecting a first amino acid to a second amino acid, wherein the first and second amino acids are selected from amino acids corresponding to the following locations of amino acids: 2 and 9; 9 and 13; 13 and 17; 14 and 18; 14 and 21; 15 and 19; 16 and 23; 17 and 21; 17 and 24; 18 and 22; 19 and 23; 19 and 26; 22 and 26; 23 and 27; and 24 and 28 of amino acids 1-29 of Human Growth Hormone-Release Hormone (GHRH 1-29).

2. The peptidomimetic macrocycle or the pharmaceutically-acceptable salt thereof of claim 1, wherein the amino acid sequence of said peptidomimetic macrocycle or the pharmaceutically-acceptable salt thereof is at least about 60% identical to an amino acid sequence of Table 1a, 1b, 2a, 2b, or 2c.

3. (canceled)

4. The peptidomimetic macrocycle or the pharmaceutically-acceptable salt thereof of claim 1, wherein the peptidomimetic macrocycle or the pharmaceutically-acceptable salt thereof is attached to a ghrelin agonist.

5. The peptidomimetic macrocycle or the pharmaceutically-acceptable salt thereof of claim 1, wherein the amino acid sequence of said peptidomimetic macrocycle or the pharmaceutically-acceptable salt thereof is at least about identical to GHRH 1-29.

6. The peptidomimetic macrocycle or the pharmaceutically-acceptable salt thereof of claim 1, wherein the amino acid sequence of said peptidomimetic macrocycle or the pharmaceutically-acceptable salt thereof is at least about identical to an amino acid sequence of Table 1a, 1b, 2a, 2b, or 2c.

7. The peptidomimetic macrocycle or the pharmaceutically-acceptable salt thereof of claim 1, wherein the amino acid sequence of said peptidomimetic macrocycle or the pharmaceutically-acceptable salt thereof is at least about 60% identical to an amino acid sequence of Table 2c.

8. The peptidomimetic macrocycle or the pharmaceutically-acceptable salt thereof of claim 1, wherein the macrocycle-forming linker connects amino acids corresponding to amino acid locations 13 and 17 of amino acids 1-29 of GHRH 1-29.

9. The peptidomimetic macrocycle or the pharmaceutically-acceptable salt thereof of claim 1, wherein the macrocycle-forming linker connects amino acids corresponding to amino acid locations 12 and 19 of amino acids 1-29 of GHRH 1-29.

10. The peptidomimetic macrocycle or the pharmaceutically-acceptable salt thereof of claim 1, wherein the peptidomimetic macrocycle or the pharmaceutically-acceptable salt thereof has no more than one macrocycle-forming linker.

11. The peptidomimetic macrocycle or the pharmaceutically-acceptable salt thereof of claim 1, comprising no more than two macrocycle-forming linkers.

12. (canceled)

13. The peptidomimetic macrocycle or the pharmaceutically-acceptable salt thereof of claim 1, having Formula (I): 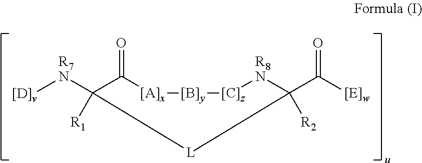
or a pharmaceutically-acceptable salt thereof wherein:
each A, C, D, and E is independently an amino acid;
each B is independently an amino acid 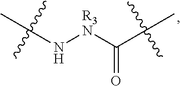
[—NH-L₃-CO—], [—NH-L₃-SO₂—], or [—NH-L₃-];
wherein A, B, C, D, and E, taken together with the crosslinked amino acids connected by the macrocycle-forming linker L, form the amino acid sequence of the peptidomimetic macrocycle;
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of the D or E amino acids;
each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker;
each L₃ is independently alkylene, alkenylene, alkynylene, heteroalkylene, cycloalkylene,
each R₄ is independently alkylene, alkenylene, alkynylene, heteroalkylene, cycloalkylene, heterocycloalkylene, arylene, or heteroarylene;
each K is independently O, S, SO, SO₂, CO, CO₂ or CONR₃;
each R₅ is independently halogen, alkyl, —OR₆, —N(R₆)₂, —SR₆, —SOR₆, —SO₂—R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v and w is independently an integer from 0-1000;
u is an integer from 1-10;
each x, y and z is independently an integer from 0-10; and
each n is independently an integer from 1-5.

14.-16. (canceled)

17. The peptidomimetic macrocycle or the pharmaceutically-acceptable salt thereof of claim 13, wherein the sum of x+y+z is 2, 3, 5 or 6.

18.-21. (canceled)

22. The peptidomimetic macrocycle or the pharmaceutically-acceptable salt thereof of claim 17, wherein x+y+z=6.

23. (canceled)

24. A peptidomimetic macrocycle having Formula (Ib): 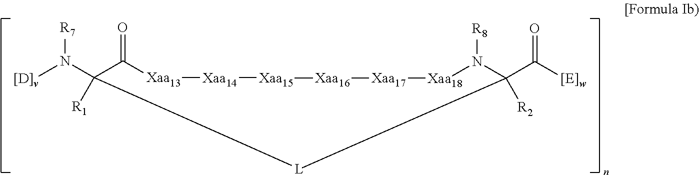
or a pharmaceutically-acceptable salt thereof wherein:
each of Xaa₃, Xaa₁₄, Xaa₁₅, Xaa₁₆, Xaa₁₇, and Xaa₁₈ is independently an amino acid, wherein at least one, two, three, four, five, or each of Xaa₁₃, Xaa₁₄, Xaa₁₅, Xaa₁₆, Xaa₁₇, and Xaa₁₈, are the same amino acid as the amino acid at the corresponding position of the sequence Xaa₁₂-Val₃-Leu₁₄-Ala/Gly₁₅-Gln/Ala₁₆-Leu₁₇-Ser₁₈-Xaa₁₉, where each of Xaa₁₂ and Xaa₁₉ is independently an amino acid (SEQ ID NO: 144);
each D and E is independently an amino acid;
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or forms a macrocycle-forming linker L′ connected to the alpha position of one of the D or E amino acids;
each L and L′ is independently a macrocycle-forming linker;
each R₃ is independently —H, alkyl, alkenyl, alkynyl, arylalkyl, heteroalkyl, cycloalkyl, heterocycloalkyl, cycloalkylalkyl, cycloaryl, or heterocycloaryl, optionally substituted with R₅;
each R₅ is independently halogen, alkyl, —OR₆, —N(R₆)₂, —SR₆, —SOR₆, —SO₂R, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
each R₈ is independently —H, alkyl, alkenyl, alknyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
each v is independently an integer from 1-1000;
each w is independently an integer from 1-1000; and
u is an integer from 1-100.

25. The peptidomimetic macrocycle or the pharmaceutically-acceptable salt thereof of claim 13, wherein each L and L′ is independently a macrocycle-forming linker of the formula -L₁-L₂-, 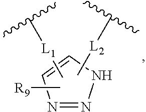
or -L₁-S-L₂-S-L₃-;
each L₁, L₂ and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂ or CONR₃;
each R₉ is independently absent, H, alkyl, alkenyl, alkynyl, aryl, cycloalkyl, cycloalkenyl, heteroaryl, or heterocyclyl group, unsubstituted or optionally substituted with Rₐ and/or R_{b};
each Rₐ and R_{b}, is independently alkyl, OCH₃, CF₃, NH₂, CH₂NH₂, F, Br, I,

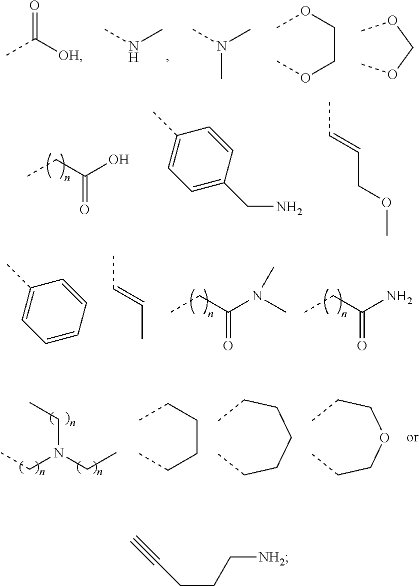

26. (canceled)

27. The peptidomimetic macrocycle or the pharmaceutically-acceptable salt thereof of claim 13, wherein u is 1.

28. The peptidomimetic macrocycle or the pharmaceutically-acceptable salt thereof of claim 13, wherein u is 2.

29. The peptidomimetic macrocycle or the pharmaceutically-acceptable salt thereof of claim 28, having the Formula: 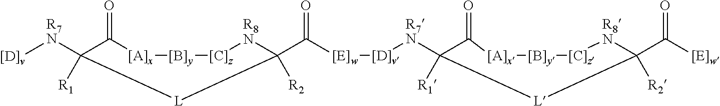
wherein:
each A, C, D, and E is independently an amino acid;
each B is independently an amino acid, 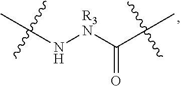
[—NH-L₃-CO—], [—NH-L₃-SO₂—], or [—NH-L₃-];
L′ is a macrocycle-forming linker of the formula-L₁′-L₂′-;
and wherein A, B, C, D, and E, taken together with the crosslinked amino acids connected by the macrocycle-forming linkers L and L′, form the amino acid sequence of the peptidomimetic macrocycle;
each R₁′ and R₂′ is independently —H, alkyl, alkenyl, alknyl, arylalkyl, cycloalkyl, cycloalkylalkyl, heteroalkyl, or heterocycloalkyl, unsubstituted or substituted with halo-;
each L₁′, L₂′, and L₃ is independently alkylene, alkenylene, alkynylene, heteroalkylene, cycloalkylene, heterocycloalkylene, arylene, heteroarylene, or [—R₄—K—R₄-]ₙ, each being optionally substituted with R₅;
each K is independently O, S, SO, SO₂, CO, CO₂, or CONR₃;
R₇′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each v′ and w′ is independently an integer from 1-100; and
each x′, y′ and z′ is independently an integer from 0-10.

30. The peptidomimetic macrocycle or the pharmaceutically-acceptable salt thereof of claim 29, wherein the sum of x′+y′+z′ is 2, 3, 5 or 6.

31.-32. (canceled)

33. The peptidomimetic macrocycle or the pharmaceutically-acceptable salt thereof of claim 13, wherein each v is independently an integer from 0-30.

34.-36. (canceled)

37. The peptidomimetic macrocycle or the pharmaceutically-acceptable salt thereof of claim 13, wherein each w is independently an integer from 0-30.

38.-42. (canceled)

43. The peptidomimetic macrocycle or the pharmaceutically-acceptable salt thereof of claim 25, wherein each L₁ and L₂ is independently alkylene, alkenylene, alkynylene, heteroalkylene, cycloalkylene, heterocycloalkylene, cycloarylene, or heterocycloarylene, each being optionally substituted with R₅.

44. The peptidomimetic macrocycle or the pharmaceutically-acceptable salt thereof of claim 25, wherein each L₁ and L₂ is independently alkylene.

45. The peptidomimetic macrocycle or the pharmaceutically-acceptable salt thereof of claim 25, wherein each L₁ and L₂ is independently alkenylene.

46. The peptidomimetic macrocycle or the pharmaceutically-acceptable salt thereof of claim 45, wherein each L₁ and L₂ is independently C₃-C₁₀ alkylene or C₃-C₁₀ alkenylene.

47. The peptidomimetic macrocycle or the pharmaceutically-acceptable salt thereof of claim 46, wherein each L₁ and L₂ is independently C₃-C₆ alkylene or C₃-C₆ alkenylene.

48.-51. (canceled)

52. The peptidomimetic macrocycle or the pharmaceutically-acceptable salt thereof of claim 13, wherein each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo.

53. The peptidomimetic macrocycle or the pharmaceutically-acceptable salt thereof of claim 13, wherein R₁ and R₂ are H.

54. The peptidomimetic macrocycle or the pharmaceutically-acceptable salt thereof of claim 13, wherein R₁ and R₂ are alkyl.

55. The peptidomimetic macrocycle or the pharmaceutically-acceptable salt thereof of claim 54, wherein R₁ and R₂ are methyl.

56. The peptidomimetic macrocycle or the pharmaceutically-acceptable salt thereof of claim 13, wherein L is

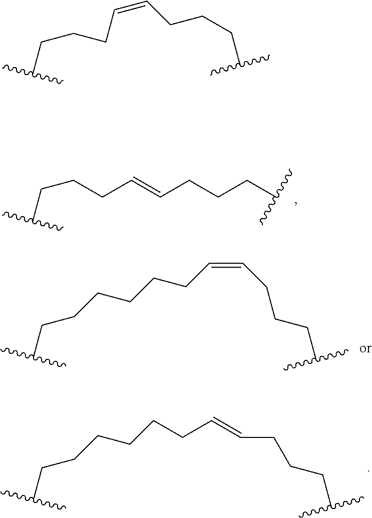 [SMILES: *CCC/C=C\CCC* ; *CCC/C=C/CCC* ; *CCC/C=C\CCCCCC* ; *CCC/C=C/CCCCCC*]

57. The peptidomimetic macrocycle or the pharmaceutically-acceptable salt thereof of claim 13, wherein L is

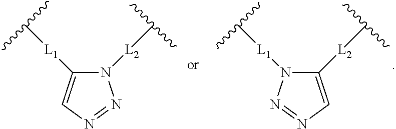

58.-112. (canceled)

113. The peptidomimetic macrocycle or the pharmaceutically-acceptable salt thereof of claim 1, wherein the amino acid sequence of the peptidomimetic macrocycle or the pharmaceutically-acceptable salt thereof is at least about 80% identical to an amino acid sequence selected from the group consisting of the amino acid sequences in Table 1a, 1b, 2a, 2b, or 2c.

114. The peptidomimetic macrocycle or the pharmaceutically-acceptable salt thereof of claim 1, wherein the amino acid sequence of the peptidomimetic macrocycle or the pharmaceutically-acceptable salt thereof is at least about 90% identical to an amino acid sequence selected from the group consisting of the amino acid sequences in Table 1a, 1b, 2a, 2b, or 2c.

115. The peptidomimetic macrocycle or the pharmaceutically-acceptable salt thereof of claim 1, wherein the amino acid sequence of the peptidomimetic macrocycle or the pharmaceutically-acceptable salt thereof is selected from the group consisting of the amino acid sequences in Table 1a, 1b, 2a, 2b, or 2c.

116.-118. (canceled)

119. The peptidomimetic macrocycle or the pharmaceutically-acceptable salt thereof of claim 1, wherein the peptidomimetic macrocycle or the pharmaceutically-acceptable salt thereof comprises a helix.

120. The peptidomimetic macrocycle or the pharmaceutically-acceptable salt thereof of claim 1, wherein the peptidomimetic macrocycle or the pharmaceutically-acceptable salt thereof comprises an α-helix.

121. The peptidomimetic macrocycle or the pharmaceutically-acceptable salt thereof of claim 1, wherein the peptidomimetic macrocycle or the pharmaceutically-acceptable salt thereof comprises an α,α-disubstituted amino acid.

122. The peptidomimetic macrocycle or the pharmaceutically-acceptable salt thereof of claim 121, wherein each amino acid connected by the macrocycle-forming linker is an α,α-disubstituted amino acid.

123.-139. (canceled)


SEQUENCE LISTING (144 sequences)

SEQ ID NO: 1 - protein, 44 aa, Artificial Sequence
  Description of Artificial Sequence Synthetic polypeptide
YADAIFTNSYRKVLGQLSARKLLQDIMSRQQGESNQERGARARL

SEQ ID NO: 2 - protein, 44 aa, Artificial Sequence; identical to SEQ ID NO: 1
  Description of Artificial Sequence Synthetic polypeptide

SEQ ID NO: 3 - protein, 32 aa, Artificial Sequence
  Description of Artificial Sequence Synthetic polypeptide
YADAIFTNSYRKVLGQLSARKLLQDIMSRQQG

SEQ ID NO: 4 - protein, 32 aa, Artificial Sequence; identical to SEQ ID NO: 3
  Description of Artificial Sequence Synthetic polypeptide

SEQ ID NO: 5 - protein, 29 aa, Artificial Sequence
  Description of Artificial Sequence Synthetic peptide
YADAIFTNSYRKVLGQLSARKLLQDIMSR

SEQ ID NO: 6 - protein, 29 aa, Artificial Sequence
  Description of Artificial Sequence Synthetic peptide
YADAIFTASYRKVLGQLSARALLQAILSR

SEQ ID NO: 7 - protein, 29 aa, Artificial Sequence
  Description of Artificial Sequence Synthetic peptide
YADAIFTNSYRAVLGQLSARKLLQDILSR

SEQ ID NO: 8 - protein, 29 aa, Artificial Sequence
  Description of Artificial Sequence Synthetic peptide
YADAIFTASYRAVLAQLSARKALQDILSR

SEQ ID NO: 9 - protein, 29 aa, Artificial Sequence
  Description of Artificial Sequence Synthetic peptide
YADAIFTASYRKVLAQLSARKALQDILSR

SEQ ID NO: 10 - protein, 29 aa, Artificial Sequence
  Description of Artificial Sequence Synthetic peptide
YADAIFTASYRAVLAALSARKALQDILSR

SEQ ID NO: 11 - protein, 29 aa, Artificial Sequence
  Description of Artificial Sequence Synthetic peptide
YADAAFTASYRAVLAQLSARKALQDILSR

SEQ ID NO: 12 - protein, 29 aa, Artificial Sequence
  Description of Artificial Sequence Synthetic peptide
YADAIFTASYRKVLAQLSARKALQDILSR

SEQ ID NO: 13 - protein, 29 aa, Artificial Sequence
  Description of Artificial Sequence Synthetic peptide
YADAIFTASYRKVLAQLSARKALQDILSR

SEQ ID NO: 14 - protein, 29 aa, Artificial Sequence
  Description of Artificial Sequence Synthetic peptide
YADAIFTASYRKVLAQLSARAALQAILSR

SEQ ID NO: 15 - protein, 29 aa, Artificial Sequence
  Description of Artificial Sequence Synthetic peptide
YADAIFTASYRKVLAQLAARKALQAILSR

SEQ ID NO: 16 - protein, 29 aa, Artificial Sequence
  Description of Artificial Sequence Synthetic peptide
YADAIFTASYRKVLAQLSARAALQDILAR

SEQ ID NO: 17 - protein, 29 aa, Artificial Sequence
  Description of Artificial Sequence Synthetic peptide
YADAIFTASYRKVLAQLSARKALQDILSA

SEQ ID NO: 18 - protein, 31 aa, Artificial Sequence
  Description of Artificial Sequence Synthetic polypeptide
PPYADAIFTASYRAVLAQLSARKALQDILSR

SEQ ID NO: 19 - protein, 29 aa, Artificial Sequence
  Description of Artificial Sequence Synthetic peptide
YADAIFTASYRAVLAQLSARKALQDILSR

SEQ ID NO: 20 - protein, 29 aa, Artificial Sequence
  Description of Artificial Sequence Synthetic peptide
YADAIFTASYRAVLAALSARKALQDILSR

SEQ ID NO: 21 - protein, 32 aa, Artificial Sequence
  Description of Artificial Sequence Synthetic polypeptide
YADAIFTASYRAVLAQLSARKALQDILSREEE

SEQ ID NO: 22 - protein, 28 aa, Artificial Sequence
  Description of Artificial Sequence Synthetic peptide
YADAIFTASYRAVLAQLSARKALQDILS

SEQ ID NO: 23 - protein, 29 aa, Artificial Sequence
  Description of Artificial Sequence Synthetic peptide
YADAIFTASYRAVLAQLSARKALQAILSR

SEQ ID NO: 24 - protein, 29 aa, Artificial Sequence
  Description of Artificial Sequence Synthetic peptide
YADAIFTASYRAVLAQLSARKALQDALSR

SEQ ID NO: 25 - protein, 29 aa, Artificial Sequence
  Description of Artificial Sequence Synthetic peptide
YADAIFTASYRAVLAQLSARKALADILSR

SEQ ID NO: 26 - protein, 29 aa, Artificial Sequence
  Description of Artificial Sequence Synthetic peptide
YADAIFTASYRAVLAQLSARKALQDIASR

SEQ ID NO: 27 - protein, 29 aa, Artificial Sequence
  Description of Artificial Sequence Synthetic peptide
YADAIFTASYRAVLAQLSARKXLQDILSR

SEQ ID NO: 28 - protein, 29 aa, Artificial Sequence
  Description of Artificial Sequence Synthetic peptide
YADAIFTXSYRAVLAQLSARKXLQDILSR

SEQ ID NO: 29 - protein, 29 aa, Artificial Sequence
  Description of Artificial Sequence Synthetic peptide
YADAIFTXSYRAVLAQLSARKALXDILSR

SEQ ID NO: 30 - protein, 29 aa, Artificial Sequence
  Description of Artificial Sequence Synthetic peptide
YADAIFTASYRAVLAQLSARKALQDISSR

SEQ ID NO: 31 - protein, 29 aa, Artificial Sequence
  Description of Artificial Sequence Synthetic peptide
YADAIFTNSYRKVLGQLSARKLLQDILSR

SEQ ID NO: 32 - protein, 30 aa, Artificial Sequence
  Description of Artificial Sequence Synthetic polypeptide
AYADAIFTASYRKVLAQLSARKALQDILSR

SEQ ID NO: 33 - protein, 29 aa, Artificial Sequence
  Description of Artificial Sequence Synthetic peptide
YADAAFTAAYRKVLAQLSARKALQDILSR

SEQ ID NO: 34 - protein, 29 aa, Artificial Sequence
  Description of Artificial Sequence Synthetic peptide
YADAIFTAAYRKALAQLSARKALQDILSR

SEQ ID NO: 35 - protein, 29 aa, Artificial Sequence
  Description of Artificial Sequence Synthetic peptide
YADAIFTASYRKVAAQLAARKALQDILSR

SEQ ID NO: 36 - protein, 29 aa, Artificial Sequence
  Description of Artificial Sequence Synthetic peptide
YADAIFTASYRKVLAQLSARKALQDILSR

SEQ ID NO: 37 - protein, 29 aa, Artificial Sequence
  Description of Artificial Sequence Synthetic peptide
YADAIFTASYRKVLAQASARAALQDILSR

SEQ ID NO: 38 - protein, 29 aa, Artificial Sequence
  Description of Artificial Sequence Synthetic peptide
YADAIFTASYRKVLAQLAARKALQDILSR

SEQ ID NO: 39 - protein, 29 aa, Artificial Sequence
  Description of Artificial Sequence Synthetic peptide
YADAIFTASYRKVLAQLSARKAAQDILSR

SEQ ID NO: 40 - protein, 29 aa, Artificial Sequence
  Description of Artificial Sequence Synthetic peptide
YADAIFTASYRKVLAQLSARKALQDALSR

SEQ ID NO: 41 - protein, 29 aa, Artificial Sequence
  Description of Artificial Sequence Synthetic peptide
YADAIFTASYRKVLAQLSARKAAQDIASR

SEQ ID NO: 42 - protein, 29 aa, Artificial Sequence
  Description of Artificial Sequence Synthetic peptide
YADAIFTASYRKVLAQLSARKALADILAR

SEQ ID NO: 43 - protein, 29 aa, Artificial Sequence
  Description of Artificial Sequence Synthetic peptide
YADAIFTASYRKVLAQLSARKALQAILSA

SEQ ID NO: 44 - protein, 29 aa, Artificial Sequence
  Description of Artificial Sequence Synthetic peptide
YADAIFTAAYRKVLAQLSARKALQDILSR

SEQ ID NO: 45 - protein, 29 aa, Artificial Sequence
  Description of Artificial Sequence Synthetic peptide
YADAIFTASYRKVAAQLSARAALQDILSR

SEQ ID NO: 46 - protein, 29 aa, Artificial Sequence
  Description of Artificial Sequence Synthetic peptide
YADAIFTASYRKVLAALSARKAAQDILSR

SEQ ID NO: 47 - protein, 29 aa, Artificial Sequence
  Description of Artificial Sequence Synthetic peptide
YADAIFTASYRKVLAQASARKALADILSR

SEQ ID NO: 48 - protein, 29 aa, Artificial Sequence
  Description of Artificial Sequence Synthetic peptide
YADAIFTASYRKVLAQLSARKALQDALSR

SEQ ID NO: 49 - protein, 29 aa, Artificial Sequence
  Description of Artificial Sequence Synthetic peptide
YADAIFTNSYRKVLAQLSARKALQDILSR

SEQ ID NO: 50 - protein, 29 aa, Artificial Sequence
  Description of Artificial Sequence Synthetic peptide
YADAIFTASYRKVLAQLSARKALQDILSR

SEQ ID NO: 51 - protein, 29 aa, Artificial Sequence
  Description of Artificial Sequence Synthetic peptide
YADAIFTASYRKVLAQLSARKALQDILSR

SEQ ID NO: 52 - protein, 29 aa, Artificial Sequence
  Description of Artificial Sequence Synthetic peptide
YADAIFTASYRKVLAQLSARKALQDILSR

SEQ ID NO: 53 - protein, 28 aa, Artificial Sequence
  Description of Artificial Sequence Synthetic peptide
ADAIFTASYRKVLAQLSARKALQDILSR

SEQ ID NO: 54 - protein, 29 aa, Artificial Sequence
  Description of Artificial Sequence Synthetic peptide
YADAIFTASYRKVLAQLSARKALQDILSR

SEQ ID NO: 55 - protein, 29 aa, Artificial Sequence
  Description of Artificial Sequence Synthetic peptide
YADAIFTASYRKVLAQLSARKALQDILSR

SEQ ID NO: 56 - protein, 29 aa, Artificial Sequence
  Description of Artificial Sequence Synthetic peptide
YADAIFTXSYRKVLAQLSARKALQDILSR

SEQ ID NO: 57 - protein, 29 aa, Artificial Sequence
  Description of Artificial Sequence Synthetic peptide
YADAIFTTSYRKVLAQLSARKALQDILSR

SEQ ID NO: 58 - protein, 29 aa, Artificial Sequence
  Description of Artificial Sequence Synthetic peptide
YADAIFTQSYRKVLAQLSARKALQDILSR

SEQ ID NO: 59 - protein, 29 aa, Artificial Sequence
  Description of Artificial Sequence Synthetic peptide
YADAIFTASYRKVLXQLSARKALQDILSR

SEQ ID NO: 60 - protein, 29 aa, Artificial Sequence
  Description of Artificial Sequence Synthetic peptide
YADAIFTASYRKVLAQLSARKALQDILDR

SEQ ID NO: 61 - protein, 29 aa, Artificial Sequence
  Description of Artificial Sequence Synthetic peptide
YADAIFTASYRKVLAQLSARKALQDILSR

SEQ ID NO: 62 - protein, 29 aa, Artificial Sequence
  Description of Artificial Sequence Synthetic peptide
YADAIFTASYXKVLAQLSARKALQDILSR

SEQ ID NO: 63 - protein, 29 aa, Artificial Sequence
  Description of Artificial Sequence Synthetic peptide
YADAIFTASYRKVLAQLSAXKALQDILSR

SEQ ID NO: 64 - protein, 29 aa, Artificial Sequence
  Description of Artificial Sequence Synthetic peptide
YADAIFTASYRKVLAQLSARKALQDILSX

SEQ ID NO: 65 - protein, 29 aa, Artificial Sequence
  Description of Artificial Sequence Synthetic peptide
YADAIFTASYRKVLAQLSARKALQDILSR

SEQ ID NO: 66 - protein, 29 aa, Artificial Sequence
  Description of Artificial Sequence Synthetic peptide
YADAIFTASYRKVLAQLSARKALQDILSR

SEQ ID NO: 67 - protein, 29 aa, Artificial Sequence
  Description of Artificial Sequence Synthetic peptide
YADAIFTXSYRKVLAQLSARKXLQDILSR

SEQ ID NO: 68 - protein, 29 aa, Artificial Sequence
  Description of Artificial Sequence Synthetic peptide
YADAIFTXSYRKVLAQLSARKALXDILSR

SEQ ID NO: 69 - protein, 29 aa, Artificial Sequence
  Description of Artificial Sequence Synthetic peptide
YADAIFTASYRKVLAQLXARKALADILSR

SEQ ID NO: 70 - protein, 29 aa, Artificial Sequence
  Description of Artificial Sequence Synthetic peptide
YADAIFTXSYRKVLAQLXARKALXDILSR

SEQ ID NO: 71 - protein, 29 aa, Artificial Sequence
  Description of Artificial Sequence Synthetic peptide
YADAIFTNSYRKVLGQLSARKLLQDIMSR

SEQ ID NO: 72 - protein, 29 aa, Artificial Sequence
  Description of Artificial Sequence Synthetic peptide
YADAIFTASYRKALAQASARKALQDILSR

SEQ ID NO: 73 - protein, 29 aa, Artificial Sequence
  Description of Artificial Sequence Synthetic peptide
YADAIFTQSYRKALAQASARKALQDILSR

SEQ ID NO: 74 - protein, 29 aa, Artificial Sequence
  Description of Artificial Sequence Synthetic peptide
YADAIFTXSYRKALAQASARKALQDILSR

SEQ ID NO: 75 - protein, 29 aa, Artificial Sequence
  Description of Artificial Sequence Synthetic peptide
YIDAIFTQSYRKALAQASARKALQDILSR

SEQ ID NO: 76 - protein, 29 aa, Artificial Sequence
  Description of Artificial Sequence Synthetic peptide
YAPAIFTQSYRKALAQASARKALQDILSR

SEQ ID NO: 77 - protein, 29 aa, Artificial Sequence
  Description of Artificial Sequence Synthetic peptide
FADAIFTQSYRKALAQASARKALQDILSR

SEQ ID NO: 78 - protein, 29 aa, Artificial Sequence
  Description of Artificial Sequence Synthetic peptide
FADAIFTQSYRKALAQASARKALQDILSR

SEQ ID NO: 79 - protein, 30 aa, Artificial Sequence
  Description of Artificial Sequence Synthetic polypeptide
PYADAIFTQSYRKALAQASARKALQDILSR

SEQ ID NO: 80 - protein, 30 aa, Artificial Sequence
  Description of Artificial Sequence Synthetic polypeptide
KYADAIFTQSYRKALAQASARKALQDILSR

SEQ ID NO: 81 - protein, 28 aa, Artificial Sequence
  Description of Artificial Sequence Synthetic peptide
ADAIFTQSYRKALAQASARKALQDILSR

SEQ ID NO: 82 - protein, 29 aa, Artificial Sequence
  Description of Artificial Sequence Synthetic peptide
YADAIFTQSYRKALAQASARKALQDILSR

SEQ ID NO: 83 - protein, 29 aa, Artificial Sequence
  Description of Artificial Sequence Synthetic peptide
YADAIFTQSYRKALAQASARKALQDILSR

SEQ ID NO: 84 - protein, 29 aa, Artificial Sequence
  Description of Artificial Sequence Synthetic peptide
YADAIFTQSYRKALAQASARKALQDILSR

SEQ ID NO: 85 - protein, 29 aa, Artificial Sequence
  Description of Artificial Sequence Synthetic peptide
YADAIFTQSYRKALAQASARKALQDILSR

SEQ ID NO: 86 - protein, 29 aa, Artificial Sequence
  Description of Artificial Sequence Synthetic peptide
YADAIFTQSYRKALAQASARKALQDILSR

SEQ ID NO: 87 - protein, 29 aa, Artificial Sequence
  Description of Artificial Sequence Synthetic peptide
YADAIFTQSYRKALAQASARKALQDILSR

SEQ ID NO: 88 - protein, 29 aa, Artificial Sequence
  Description of Artificial Sequence Synthetic peptide
YADAIFTQSYRKALAQASARKALQDILSR

SEQ ID NO: 89 - protein, 29 aa, Artificial Sequence
  Description of Artificial Sequence Synthetic peptide
YADAIFTQSYRKALAQASARKALQDILSR

SEQ ID NO: 90 - protein, 29 aa, Artificial Sequence
  Description of Artificial Sequence Synthetic peptide
YADAIFTQSYRKALAQASARKALQDILSR

SEQ ID NO: 91 - protein, 29 aa, Artificial Sequence
  Description of Artificial Sequence Synthetic peptide
YADAIFTQSYRKALAQASARKALQDILSR

SEQ ID NO: 92 - protein, 29 aa, Artificial Sequence
  Description of Artificial Sequence Synthetic peptide
YADAIFTQSYRKALAQASARKALQDILSR

SEQ ID NO: 93 - protein, 29 aa, Artificial Sequence
  Description of Artificial Sequence Synthetic peptide
YADAIFTQSYRKALAQASARKALQDILSR

SEQ ID NO: 94 - protein, 29 aa, Artificial Sequence
  Description of Artificial Sequence Synthetic peptide
YADAIFTASYRKALAQASARKALQDILSR

SEQ ID NO: 95 - protein, 29 aa, Artificial Sequence
  Description of Artificial Sequence Synthetic peptide
YADAIFTQSYRKALAAASARKALQDILSR

SEQ ID NO: 96 - protein, 29 aa, Artificial Sequence
  Description of Artificial Sequence Synthetic peptide
YADAIFTQSYRKALAEASARKALQDILSR

SEQ ID NO: 97 - protein, 29 aa, Artificial Sequence
  Description of Artificial Sequence Synthetic peptide
YADAIFTQSYRKALALASARKALQDILSR

SEQ ID NO: 98 - protein, 29 aa, Artificial Sequence
  Description of Artificial Sequence Synthetic peptide
YADAIFTQSYRKALASASARKALQDILSR

SEQ ID NO: 99 - protein, 29 aa, Artificial Sequence
  Description of Artificial Sequence Synthetic peptide
YADAIFTQSYRKXLAQXSARKALQDILSR

SEQ ID NO: 100 - protein, 29 aa, Artificial Sequence
  Description of Artificial Sequence Synthetic peptide
YADAIFTQSYRKXLAQXSARKALQDILSR

SEQ ID NO: 101 - protein, 29 aa, Artificial Sequence
  Description of Artificial Sequence Synthetic peptide
YADAIFTQSYRKXLAQXSARKALQDILSR

SEQ ID NO: 102 - protein, 29 aa, Artificial Sequence
  Description of Artificial Sequence Synthetic peptide
YADAIFTQSYRKXLAQXSARKALQDILSR

SEQ ID NO: 103 - protein, 29 aa, Artificial Sequence
  Description of Artificial Sequence Synthetic peptide
YADAIFTQSYRKKLAQESARKALQDILSR

SEQ ID NO: 104 - protein, 27 aa, Artificial Sequence
  Description of Artificial Sequence Synthetic peptide
YADAIFTQSYRKLAQSARKALQDILSR

SEQ ID NO: 105 - protein, 29 aa, Artificial Sequence
  Description of Artificial Sequence Synthetic peptide
YADAIFTQSYRKALAQASARKALQDILSR

SEQ ID NO: 106 - protein, 29 aa, Artificial Sequence
  Description of Artificial Sequence Synthetic peptide
YADAIFTQSYRKALAQASARKALQDILSR

SEQ ID NO: 107 - protein, 29 aa, Artificial Sequence
  Description of Artificial Sequence Synthetic peptide
YADAIFTQSYRKALAQASARKALQDILSR

SEQ ID NO: 108 - protein, 29 aa, Artificial Sequence
  Description of Artificial Sequence Synthetic peptide
YADAIFTQSYRKALAAASARKALQDILSR

SEQ ID NO: 109 - protein, 29 aa, Artificial Sequence
  Description of Artificial Sequence Synthetic peptide
YADAIFTQSYRKALAEASARKALQDILSR

SEQ ID NO: 110 - protein, 29 aa, Artificial Sequence
  Description of Artificial Sequence Synthetic peptide
YADAIFTQSYRKALALASARKALQDILSR

SEQ ID NO: 111 - protein, 29 aa, Artificial Sequence
  Description of Artificial Sequence Synthetic peptide
YADAIFTQSYRKALASASARKALQDILSR

SEQ ID NO: 112 - protein, 29 aa, Artificial Sequence
  Description of Artificial Sequence Synthetic peptide
YADAIFTQSYRKALAQASARKALQDILSR

SEQ ID NO: 113 - protein, 29 aa, Artificial Sequence
  Description of Artificial Sequence Synthetic peptide
YIDAIFTASYRKALAQASARKALQDILSR

SEQ ID NO: 114 - protein, 29 aa, Artificial Sequence
  Description of Artificial Sequence Synthetic peptide
YAPAIFTASYRKALAQASARKALQDILSR

SEQ ID NO: 115 - protein, 29 aa, Artificial Sequence
  Description of Artificial Sequence Synthetic peptide
FADAIFTASYRKALAQASARKALQDILSR

SEQ ID NO: 116 - protein, 29 aa, Artificial Sequence
  Description of Artificial Sequence Synthetic peptide
FADAIFTASYRKALAQASARKALQDILSR

SEQ ID NO: 117 - protein, 30 aa, Artificial Sequence
  Description of Artificial Sequence Synthetic polypeptide
PYADAIFTASYRKALAQASARKALQDILSR

SEQ ID NO: 118 - protein, 30 aa, Artificial Sequence
  Description of Artificial Sequence Synthetic polypeptide
KYADAIFTASYRKALAQASARKALQDILSR

SEQ ID NO: 119 - protein, 28 aa, Artificial Sequence
  Description of Artificial Sequence Synthetic peptide
ADAIFTASYRKALAQASARKALQDILSR

SEQ ID NO: 120 - protein, 29 aa, Artificial Sequence
  Description of Artificial Sequence Synthetic peptide
YADAIFTASYRKALAQASARKALQDILSR

SEQ ID NO: 121 - protein, 29 aa, Artificial Sequence
  Description of Artificial Sequence Synthetic peptide
YADAIFTASYRKALAQASARKALQDILSR

SEQ ID NO: 122 - protein, 29 aa, Artificial Sequence
  Description of Artificial Sequence Synthetic peptide
YADAIFTASYRKALAQASARKALQDILSR

SEQ ID NO: 123 - protein, 29 aa, Artificial Sequence
  Description of Artificial Sequence Synthetic peptide
YADAIFTASYRKALAQASARKALQDILSR

SEQ ID NO: 124 - protein, 29 aa, Artificial Sequence
  Description of Artificial Sequence Synthetic peptide
YADAIFTASYRKALAQASARKALQDILSR

SEQ ID NO: 125 - protein, 29 aa, Artificial Sequence
  Description of Artificial Sequence Synthetic peptide
YADAIFTASYRKALAQASARKALQDILSR

SEQ ID NO: 126 - protein, 29 aa, Artificial Sequence
  Description of Artificial Sequence Synthetic peptide
YADAIFTASYRKALAQASARKALQDILSR

SEQ ID NO: 127 - protein, 29 aa, Artificial Sequence
  Description of Artificial Sequence Synthetic peptide
YADAIFTASYRKALAQASARKALQDILSR

SEQ ID NO: 128 - protein, 29 aa, Artificial Sequence
  Description of Artificial Sequence Synthetic peptide
YADAIFTASYRKALAQASARKALQDILSR

SEQ ID NO: 129 - protein, 29 aa, Artificial Sequence
  Description of Artificial Sequence Synthetic peptide
YADAIFTASYRKALAQASARKALQDILSR

SEQ ID NO: 130 - protein, 29 aa, Artificial Sequence
  Description of Artificial Sequence Synthetic peptide
YADAIFTASYRKALAQASARKALQDILSR

SEQ ID NO: 131 - protein, 29 aa, Artificial Sequence
  Description of Artificial Sequence Synthetic peptide
YADAIFTASYRKALAQASARKALQDILSR

SEQ ID NO: 132 - protein, 30 aa, Artificial Sequence
  Description of Artificial Sequence Synthetic polypeptide
YADAIFTQSYRKALAQASARKALQDILSRK

SEQ ID NO: 133 - protein, 29 aa, Artificial Sequence
  Description of Artificial Sequence Synthetic peptide
YADAIFTQSYRKALAQASARKALQDILSR

SEQ ID NO: 134 - protein, 30 aa, Artificial Sequence; identical to SEQ ID NO: 132
  Description of Artificial Sequence Synthetic polypeptide

SEQ ID NO: 135 - protein, 29 aa, Artificial Sequence
  Description of Artificial Sequence Synthetic peptide
YADAIFTQSYRKALAQASARKALQDILSR

SEQ ID NO: 136 - protein, 30 aa, Artificial Sequence; identical to SEQ ID NO: 132
  Description of Artificial Sequence Synthetic polypeptide

SEQ ID NO: 137 - protein, 29 aa, Artificial Sequence
  Description of Artificial Sequence Synthetic peptide
YADAIFTQSYRKALAQASARKALQDILSR

SEQ ID NO: 138 - protein, 30 aa, Artificial Sequence; identical to SEQ ID NO: 132
  Description of Artificial Sequence Synthetic polypeptide

SEQ ID NO: 139 - protein, 29 aa, Artificial Sequence
  Description of Artificial Sequence Synthetic peptide
YADAIFTQSYRKALAQASARKALQDILSR

SEQ ID NO: 140 - protein, 30 aa, Artificial Sequence; identical to SEQ ID NO: 132
  Description of Artificial Sequence Synthetic polypeptide

SEQ ID NO: 141 - protein, 30 aa, Artificial Sequence; identical to SEQ ID NO: 132
  Description of Artificial Sequence Synthetic polypeptide

SEQ ID NO: 142 - protein, 30 aa, Artificial Sequence; identical to SEQ ID NO: 132
  Description of Artificial Sequence Synthetic polypeptide

SEQ ID NO: 143 - protein, 30 aa, Artificial Sequence; identical to SEQ ID NO: 132
  Description of Artificial Sequence Synthetic polypeptide

SEQ ID NO: 144 - protein, 8 aa, Artificial Sequence
  Description of Artificial Sequence Synthetic peptide
XVLXXLSX